(19) United States
(12) Patent Application Publication
(10) Pub. No.: US 2023/0381237 A1
(43) Pub. Date: Nov. 30, 2023

(54) CHIMERIC ANTIGEN RECEPTOR (CAR) T-CELL ADJUVANT THERAPIES

(71) Applicant: CytoAgents, Inc., Pittsburgh, PA (US)

(72) Inventors: Jodi CRAIGO, Cranberry Township, PA (US); Teresa WHALEN, Pittsburgh, PA (US); Heather Lynn NOTTINGHAM, Carmel Valley, CA (US); Arthur P. BERTOLINO, Holland, MI (US)

(73) Assignee: CytoAgents, Inc., Pittsburgh, PA (US)

(21) Appl. No.: 18/325,930

(22) Filed: May 30, 2023

Related U.S. Application Data
(60) Provisional application No. 63/482,448, filed on Jan. 31, 2023.
(60) Provisional application No. 63/347,221, filed on May 31, 2022.

(51) Int. Cl. A61K 35/17; A61K 31/5585; A61P 35/00; A61K 9/00

(52) U.S. Cl. CPC A61K 35/17; A61K 31/5585; A61P 35/00; A61K 9/0019

(57) ABSTRACT

Methods, compositions, and kits that can be used to treat cytokine release syndrome, ICANS, or both, associated with CAR T-cell administration are described herein. For example, pharmaceutical compositions containing beraprost, beraprost isomers, or salts thereof can be used as an adjuvant therapy with chimeric antigen receptor T cells (CAR T-cells) to treat cancer while reducing or eliminating undesired and potentially dangerous side effects.

### CROSS-REFERENCE TO RELATED APPLICATIONS

This application claims priority to U.S. Provisional Application No. 63/347,221, which was filed on May 31, 2022, entitled Chimeric Antigen Receptor (CAR) T-Cell Adjuvant Therapies, and U.S. Provisional Application No. 63/482,448, which was filed on Jan. 31, 2023, entitled Chimeric Antigen Receptor (CAR) T-Cell Adjuvant Therapies, the contents of which are incorporated by reference in its entirety.

### BACKGROUND

Chimeric antigen receptor T-cells (CAR T-cells) are genetically engineered T-cells that produce a receptor having the ability to bind to a specific protein. CAR T-cell therapies are attractive for use in immunotherapy as they can be designed and engineered to target and recognize cancer cells specifically.

T-cells are harvested and are genetically engineered prior to infusing back into the patient to attack their cancer cells. The T-cells can be obtained from the patient's blood (“autologous”) or obtained from another donor (“allogeneic”). The chimeric antigen receptor can target an antigen that is present on the surface of cancer cells in a tumor and/or an antigen present on a non-cancer cell, for example, a B cell.

One common but serious side effect of CAR T-cell therapy is cytokine release syndrome (“CRS”). The CAR-T cells cause immune cell activation (T cells, macrophages, and/or dendritic cells). The CAR T-cells and associated cells cause the release of large amounts of cytokines into the blood. The cytokines released can also cause immune hyperactivation due to prolonged activation of cytokine signaling. This can cause high fever, low blood pressure (hypotension), fatigue, headache, myalgia, nausea, capillary leakage, tachycardia, and potentially multi-organ failure, for example, liver failure and kidney damage and even result in death. CRS occurs in 50-100% of patients. A discussion of side effects can be found in the NCCN Guidelines for Patients entitled “Immunotherapy Side Effects: CAR T-Cell Therapy—English (2020) and NCCN Guidelines Version 4.2021 entitled “Management of CAR T-Cell-Related Toxicities” (2021). Immune effector cell-associated neurotoxicity syndrome (ICANS) effects can occur in more than 25% of the patients receiving CAR T-cell therapy. ICANS can manifest as headache, confusion, language disturbance, altered consciousness, seizures, cerebral edema. Virtually all patients with ICANS may have had prior CRS.

For example, with the Kymriah product from Novartis (Kymriah is a registered trademark of Novartis AG; Basel, Switzerland), CRS occurred in 79% of acute lymphocytic leukemia and 74% patients with relapsed/refractory diffuse large B cell lymphoma receiving KYMRIAH, including greater than or equal to grade 3 in 49% of patients with relapsed/refractory acute lymphocytic leukemia and in 23% of patients with relapsed/refractory diffuse large B cell lymphoma. The median time to onset was 3 days, and the median time to resolution was 8 days (a range of 1-36 days). Similarly, with the Yescarta product from Kite/Gilead (Yescarta is a registered trademark of Kite Pharma, Inc.; Santa Monica, CA, USA), CRS occurred in over 90% of patients, with 9% greater or equal to Grade 3. Among patients who died after receiving Yescarta, 4 had ongoing CRS at death. The median time to onset was 2 days (range: 1-12 days) and median duration was 7 days (range: 2-58 days).

From one perspective, the development of CRS is an indicator or diagnostic marker that the CAR T-cells are functioning to attack cancer cells. CRS develops in a delayed time after initiation of CAR T-cell administration. This lag time is very predictable and consistent, but the length of the delay is not predictable. This inability to predict if and when a patient will get CRS is challenging.

In the event of CRS or ICANS, current approved treatment includes supportive care, corticosteroids, and administration of tocilizumab, an IL-6 antagonist. While corticosteroids can be effective in treating both CRS and ICANS, there can be very serious adverse effects, including severe hypertension, glucose intolerance, susceptibility to serious infection, delayed wound healing, gastrointestinal bleeding, sepsis, and heart failure. There is also concern that corticosteroids may impair the function of the infused CAR T-cells. For this reason, corticosteroid use is often reserved for the most severe cases of CRS and ICANS. Tocilizumab, while FDA approved for the treatment of CRS, may have some drawbacks, as IL-6 has been clearly proven to promote the proliferation of T cells, and it is unclear if IL-6 blockade may impair the in vivo proliferation of CAR T-cells. Moreover, serum IL-6 levels may actually rise following tocilizumab administration, and this increase in circulating cytokines may increase the risk of vascular leak and breakdown of the BBB, which are believed to contribute to the development of ICANS. Thus, tocilizumab is generally not recommended for the treatment of ICANS, and some have postulated that its use could make the condition worse. Therefore, although helpful in a percentage of the population, the drug mechanism of action may be limited in this population for addressing CRS and ICANS. New therapeutic strategies that prevent or reduce Tocilizumab and/or steroid use are needed.

CAR T-cell therapies are powerful and effective “last resort” therapies, but there exists a need for new and improved treatments to reduce or eliminate the undesired and potentially fatal CRS side effects.

### SUMMARY

Disclosed herein are methods and compositions to administer CAR T-cell therapies while reducing or eliminating cytokine release syndrome (CRS) side effects and/or immune effector cell-associated neurotoxicity syndrome (ICANS) effects. CAR T-cell therapies can be used to treat cancer or other conditions.

Methods of treating cytokine release syndrome, ICANS, or both, associated with CAR T-cell administration can generally comprise administering CAR T-cells and a first pharmaceutical composition to a subject as an adjuvant therapy/combination therapy approach. The first pharmaceutical composition can comprise an effective amount of a prostacyclin/prostaglandin analog, such as analogs selected from the group consisting of carbaprostacyclin, beraprost, taprostene, nileprost, iloprost, cicaprost, ciprostene, treprostinil, bonsentan, uoprost, eptaloprost, or an isomer thereof, and pharmaceutically acceptable salts thereof. In some embodiments, the prostacyclin/prostaglandin analog is beraprost or a beraprost salt. The salt can be a pharmaceutically acceptable salt of beraprost such as beraprost sodium. The beraprost can be a beraprost isomer, such as beraprost CTO1681. The first pharmaceutical composition causes negligible to no reduction in CAR T-cell mediated killing, for example the reduction in CAR T-cell mediated killing is not more than about 5%.

Another method of treating cytokine release syndrome, ICANS, or both, associated with CAR T-cell administration in a subject can include administering CAR T-cells, a first pharmaceutical composition, and a second pharmaceutical composition to the subject, wherein: the first pharmaceutical composition comprises at least an effective amount of beraprost, a beraprost isomer, or a pharmaceutically acceptable salt thereof; and the second pharmaceutical composition comprises at least one corticosteroid, tocilizumab, IL-6 receptor blocker, or combinations thereof.

An additional method of treating cytokine release syndrome, ICANS, or both, associated with CAR T-cell administration can include administering CAR T-cells and a first pharmaceutical composition to the subject, wherein: the first pharmaceutical composition comprises at least an effective amount of beraprost, a beraprost isomer, or a pharmaceutically acceptable salt thereof; and the subject experiences reduced ICANS effects, Parkinsonism effects, or both, relative to a similar subject receiving administered CAR T-cells but not receiving the administered pharmaceutical composition.

A further method of treating cytokine release syndrome, ICANS, or both, associated with CAR T-cell administration in a subject can include administering CAR T-cells and a first pharmaceutical composition to the subject, wherein: the first pharmaceutical composition comprises at least an effective amount of beraprost, a beraprost isomer, or a pharmaceutically acceptable salt thereof, and the subject experiences reduced severity measurements relative to a similar subject receiving administered CAR T-cells but not receiving the administered pharmaceutical composition. In some embodiments, the subject experiences reduced severity of CRS and/or ICANS effects.

An additional method of treating cytokine release syndrome, ICANS, or both, associated with CAR T-cell administration in a subject can include administering CAR T-cells and a first pharmaceutical composition to the subject, wherein: the first pharmaceutical composition comprises at least an effective amount of beraprost, a beraprost isomer, or a pharmaceutically acceptable salt thereof, and the first pharmaceutical composition is administered once onset of CRS is detected by an increased level of one or more of cytokine MIF, IL-5, IL-17A, IL-23, IFN-γ, CXCL9/MIG, GCSF, VEGF-A, and TGF-β. Alternatively or additionally, onset of CRS can be detected by an increased level of one or more of cytokine CCL2, IL-2, IL-6, IL-8, IL-10, IFN-γ, TNF-α, CXCL9, CXCL-10, VEGF, CCL3, GCSF, and GMCSF. Alternatively, CRS onset can be detected by detection of an increased level of one or more inflammatory markers associated with CRS such as CRP (C-reactive protein) and ferritin. Alternatively, CRS onset can be detected by presentation of symptoms, such as one or more of fever, hypotension, and hypoxia.

An additional method of treating cytokine release syndrome, ICANS, or both, associated with CAR T-cell administration in a subject can comprise administering CAR T-cells and a first pharmaceutical composition to the subject, wherein: the first pharmaceutical composition comprises at least an effective amount of beraprost, a beraprost isomer, or a pharmaceutically acceptable salt thereof, the subject experiences CRS, ICANS, Parkinsonism effects, or a combination thereof, and the subject requires reduced treatment with at least one corticosteroid, tocilizumab, IL-6 receptor blocker, or combinations thereof relative to a similar subject receiving administered CAR T-cells but not receiving the administered first pharmaceutical composition.

Kits for the treatment of cytokine release syndrome, ICANS, or both, associated with CAR T-cell administration in a subject can generally comprise a first container containing a first pharmaceutical composition comprising at least an effective amount of beraprost, a beraprost isomer, or a pharmaceutically acceptable salt thereof; a second container containing a second pharmaceutical composition comprising at least one corticosteroid, tocilizumab, IL-6 receptor blocker, or combinations thereof; a third container containing CAR T-cells; and instructions for the administration of the first pharmaceutical composition, the second pharmaceutical composition, and CAR T-cells to the subject.

Additional kits can include a first container containing a first pharmaceutical composition comprising at least an effective amount of beraprost, a beraprost isomer, or a pharmaceutically acceptable salt thereof, a second container containing a second pharmaceutical composition comprising at least one corticosteroid, tocilizumab, IL-6 receptor blocker, or combinations thereof; and instructions for the administration of the first pharmaceutical composition, the second pharmaceutical composition, and CAR T-cells to a subject.

### BRIEF DESCRIPTION OF THE DRAWINGS

The foregoing and other objects, features, and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1A shows that CTO1681 does not interfere with the tumor-killing efficacy of CD19-targeting CAR T-cells in vitro (see line connected by square points) and dose-dependently lowers the levels of IL-6, a pro-inflammatory and CRS-inducing cytokine (see bar graph) when CAR T-cells and Raji cells are present at a 10:1 ratio.

FIG. 1B shows that CTO1681 does not interfere with the tumor-killing efficacy of CD19-targeting CAR T-cells in vitro (see line connected by square points) and dose-dependently lowers the levels of TNF-α, a pro-inflammatory and CRS-inducing cytokine (see bar graph) when CAR T-cells and Raji cells are present at a 10:1 ratio.

FIG. 1C shows that CTO1681 does not interfere with the tumor-killing efficacy of CD19-targeting CAR T-cells in vitro (see line connected by square points) and dose-dependently lowers the levels of IFN-γ, a pro-inflammatory and CRS-inducing cytokine (see bar graph) when CAR T-cells and Raji cells are present at a 10:1 ratio.

FIG. 1D shows IL-6 reduction upon treatment with increasing concentrations of CTO1681, across three different concentrations of CAR-T cells to Raji cells.

FIG. 1E shows TNF-α reduction upon treatment with increasing concentrations of CTO1681, across three different concentrations of CAR-T cells to Raji cells.

FIG. 1F shows IFN-7 reduction upon treatment with increasing concentrations of CTO1681, across three different concentrations of CAR-T cells to Raji cells.

FIG. 1G shows the absence of an effect of CTO1681 on CAR-T tumor killing efficacy.

FIG. 2 shows that CTO1681 does not interfere with CAR T-cell efficacy in the reduction of lymphoma tumor burden in vivo.

FIG. 3 depicts the concentration of TNF-alpha in cultured PBMCs as a function of the concentration of beraprost (E; diamonds), or single isomers of beraprost (A=BPS-314d, circle; B=BPS-315l, squares; C=BPS-315d, upward triangles; D=BPS-3141, downward triangles).

FIG. 4A depicts the plasma concentration of CTO1681 in African Green Monkeys following three times a day (TID) oral dosing at 0.008 mg/kg/day.

FIG. 4B depicts the plasma concentration of CTO1681 in African Green Monkeys following three times a day (TID) oral dosing at 0.04 mg/kg/day.

FIG. 4C depicts the plasma concentration of CTO1681 in African Green Monkeys following three times a day (TID) oral dosing at 0.2 mg/kg/day.

FIG. 4D depicts the plasma concentration of CTO1681 in African Green Monkeys following three times a day (TID) oral dosing at 0.008 mg/kg/day, 0.04 mg/kg/day, and 0.2 mg/kg/day.

FIG. 5A shows the average maximum plasma concentration (Cmax) in African Green Monkeys for each dose interval of CTO1681.

FIG. 5B shows the area under the curve (AUC) for each dose of CTO1681 in African Green Monkeys.

FIG. 6 depicts the TNF-alpha concentration in serum samples from African Green Monkeys after treatment with CTO1681.

### DEFINITIONS

As used herein, the term “about” when immediately preceding a numerical value means a range of plus or minus 10% of that value, for example, “about 50” means 45 to 55, “about 25,000” means 22,500 to 27,500, etc., unless the context of the disclosure indicates otherwise, or is inconsistent with such an interpretation.

As used herein the term “analog” refers to a compound, the presence of which results in a biological activity of a receptor that is the same as the biological activity resulting from the presence of a naturally occurring ligand for the receptor.

The terms “administer,” “administering” or “administration” as used herein refer to directly administering a compound or a composition to a subject.

As used herein, the term “effective amount” refers to an amount that results in measurable inhibition of at least one symptom or parameter of a specific disorder or pathological process. As used herein the term “therapeutically effective amount” of compositions of the application is an amount, which confers a therapeutic effect on the treated subject, at a reasonable benefit/risk ratio applicable to any medical treatment. The therapeutic effect may be objective (that is, measurable by some test or marker) or subjective (that is, subject gives an indication of or feels an effect or physician observes a change).

As used herein the term “immediate release” refers to pharmaceutical compositions that release the active ingredient within a short period of time.

As used herein the term “modified release” refers to pharmaceutical compositions that does not otherwise release the active ingredient immediately, for example it may release the active ingredient at a sustained or controlled rate over an extended period of time, or may release the active ingredient after a lag time after administration, or may be used optionally in combination with an immediate release composition. Modified release includes extended release, sustained release, and delayed release. The term “extended release” or “sustained release” as used herein is a dosage form that makes a drug available over an extended period of time after administration. The term “delayed release” as used herein is a dosage form that releases a drug at a time other than immediately upon administration.

The phrase “pharmaceutically acceptable salt(s)”, as used herein, includes those salts of compounds of the application that are safe and effective for use in mammals and that possess the desired biological activity. Pharmaceutically acceptable salts include salts of acidic or basic groups present in compounds of the application or in compounds identified pursuant to the methods of the application. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (that is, 1,1′-methylene-bis-(2-hydroxy-3-naphthoate)) salts. Certain compounds of the application can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N, N′-dibenzylethylenediamine, chloroprocaine, choline, diethanolamine, dicyclohexylamine, ethylenediamine, N-methylglucamine, and procaine.

The term “preventing” may be taken to mean to prevent a specific disorder, disease, or condition and/or prevent the reoccurrence of a specific disorder, disease, or condition.

The term “substantially pure isomer” refers to a formulation or composition wherein among various isomers of a compound a single isomer is present at about 70%, or greater or at about 80% or greater, or at about 90% or greater, or at about 95% or greater, or at about 98% or greater, or at about 99% or greater, or said compound or composition comprise only a single isomer of the compound.

As used herein the terms “treat”, “treated”, or “treating” refer to both therapeutic treatment and prophylactic or preventative measures, wherein the object is to protect against (partially or wholly) or slow down (for example, lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (that is, not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

The term “unit dosage form” refers to physically discrete units suitable as a unitary dosage for human subjects and other animals, each unit containing a predetermined quantity of active material calculated to produce the desired therapeutic effect, in association with a suitable pharmaceutical excipient.

The “weight percent” disclosed herein may be weight-to-weight percent or weight-to-volume percent, depending upon the composition.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a medical condition or disorder. Such administration encompasses co-administration of these therapeutic agents in a substantially simultaneous manner, such as in a single capsule, or dosage presentation, having a fixed ratio of active ingredients or in multiple, separate capsules for each active ingredient. In addition, such administration also encompasses use of each type of therapeutic agent in a sequential manner in the same patient, with delivery of the individual therapeutics separated by about 1-24 hours, about 1-7 days, or about 1 or more weeks. In either case, the treatment regimen will provide beneficial effects of the drug combination in treating the conditions or disorders described herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can, of course, vary. It is also to be understood that the terminology used herein is for the purpose of describing particular embodiments only and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having about 1-3 cells refers to groups having about 1, 2, or 3 cells. Similarly, a group having about 1-5 cells refers to groups having about 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

### DETAILED DESCRIPTION

This disclosure is not limited to the particular systems, devices and methods described, as these may vary. The terminology used in the description is for the purpose of describing the particular versions or embodiments only and is not intended to limit the scope.

Cytokine release syndrome (“CRS”) and/or ICANS are common but serious side effects of CAR T-cell therapy. The CAR T-cells can cause the release of large amounts of pro-inflammatory cytokines into the blood of the subject being treated with the CAR-T cells. This can cause high fever, low blood pressure (hypotension), fatigue, headache, myalgia, nausea, capillary leakage, tachycardia, and potentially liver failure and kidney damage. CRS occurs in about 50-100% of patients. Compositions and methods to overcome CRS are needed to enhance the therapeutic value of CAR T-cell therapy. Various methods, compositions, and kits are described herein for performing CAR T-cell therapies with reduced or eliminated cytokine release syndrome (CRS) effects, immune effector cell-associated neurotoxicity syndrome (ICANS) effects, Parkinsonism effects, or combinations thereof in a subject. The methods can include administration of CAR T-cells and at least one pharmaceutical composition such as a first pharmaceutical composition and/or a second pharmaceutical composition to the subject. The T-cells and T-cell therapy can be autologous or allogeneic.

In the present disclosure inventors demonstrate that the first pharmaceutical compositions comprising beraprost, a beraprost isomer, or a pharmaceutically acceptable salt of beraprost, can mitigate cytokine responses associated with CRS. In particular, the inventors observed a reduction in pro-inflammatory cytokines such as, TNF-α, and INF-7. Significantly, inventors found that beraprost, a beraprost isomer, or a pharmaceutically acceptable salt of beraprost did not interfere with CAR T-cell killing both in vitro and in vivo.

In some embodiments, administration of beraprost, a beraprost isomer, or a pharmaceutically acceptable salt thereof can mitigate the excessive release of proinflammatory cytokines, and thereby reduce or eliminate damage caused CRS and reduce or eliminate concurrent or subsequent immune effector cell-associated neurotoxicity syndrome (ICANS) effects or Parkinsonism effects. ICANS typically occurs after CRS, but sometimes can happen simultaneously. When occurring after CRS, it can be days later (for example, about 1, about 2, about 3, about 4, about 5 days or more) and sometimes even weeks later. There is some variability among subjects, even among subjects with similar genetic backgrounds.

Current standard of care (SOC) for CRS includes treatment with tocilizumab, and with corticosteroids and supportive care based on the observed CRS symptoms. Standard of care (SOC) for ICANS does not include use of tocilizumab but is a more intensive corticosteroid treatment and supportive care based on the observed ICANS symptoms. Tocilizumab, used to block IL-6 receptors, causes more IL-6 to be in circulation. The use of corticosteroids has significant limitations due to immune suppression and other deleterious downstream effects. Corticosteroids can be administered prophylactically, although this is not typical or common. Individuals given corticosteroid treatment typically must remain in the hospital for monitoring, thus extending their stay and increasing associated expense.

Hospital stays typically average about 7 to 14 days due to the negative side effects of CAR T-cell therapy. In particular, patients with CRS and ICANS symptoms require lengthy hospital stays. Use of outpatient treatments is not advised due to immunosuppressed patient populations being at an increased risk of infections.

The use of an orally provided first pharmaceutical composition, such as those containing beraprost, an isomer or salt thereof, would allow more patients to seek life-saving CAR T-cell and bispecific antibody treatments.

The onset of CRS can be detected in multiple ways. For example, onset can be detected by measuring an increase of one or more cytokines or one or more inflammatory markers. Alternatively, onset of CRS can be measured by presentation of clinical symptoms. Common symptoms include fever, hypotension, and hypoxia.

Methods, compositions, and kits can be used in therapies with reduced or eliminated effects from immune effector cell-associated neurotoxicity syndrome (ICANS), Parkinsonism, or both. The methods, compositions, and kits can also be used in therapies with reduced or eliminated severity measurements. The methods, compositions, and kits can also be used in therapies with reduced or eliminated need for corticosteroid treatment, IL-6 receptor blocker therapeutics, and other biological treatments. Beraprost compound CTO1681 could potentially replace tocilizumab as an early treatment option.

CAR T-Cell Therapy

CAR T-cell therapy can involve the infusion of T-cells that have been genetically engineered to express a CAR to reprogram the T-cells to recognize antigens expressed on the surface of target cells, for example, tumor cells. Specificity of a CAR can be from the extracellular domain of the engineered receptor, which can be derived from the antigen-binding site of an antibody. The intracellular domain can be designed to recapitulate the series of events by which T-cells are activated and can incorporate stimulatory and costimulatory domains, such as CD28 and/or 4-1BB or CD3z, to augment CAR T-cell activation, survival, and proliferation. Furthermore, CAR T-cells can multiply and differentiate into central or effector memory cells and as a “living drug” have been observed to persist in the body for 30 days and as long as four years after administration. Once activated, clonally expanded CAR T-cells release cytokines and other soluble mediators that may directly kill antigen-expressing target cells as well as normal cells.

The CAR-T cells can be any CAR T-cells, such as, but not limited to CD19 CAR T-cells, BCMA CAR T-cells and/or CD19-CD3 bispecific CAR-T cells.

Types of Cancers

In some embodiments, the CAR-T cell therapy are administered to a patient with cancer. The cancer can generally be any cancer suitable for treatment with CAR T-cell therapy. In some embodiments, the cancer can be a hematological malignancy. For example, the cancer can be B-cell lymphoma, aggressive, relapsed or refractory diffuse large B cell lymphoma (DLBCL), primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, or multiple myeloma. In some examples, the cancer is B-cell lymphoma. In some embodiments, the cancer can be a solid tumor. Cancers suitable for treatment with CAR T-cell therapy include brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, and liver metastases.

Methods of Treating Cytokine Release Syndrome, ICANS, or Both, Associated with CAR T-Cell Administration

The compounds and pharmaceutical compositions described herein may be administered at therapeutically effective dosage levels to treat the recited conditions, disorders, and diseases.

The compounds and pharmaceutical compositions described herein may be administered at prophylactically effective dosage levels to mitigate or prevent the recited conditions, disorders, and diseases.

In some embodiments, beraprost, a beraprost isomer, or a pharmaceutically acceptable salt thereof, can be used to treat CRS, ICANS or both. In some embodiments, the CRS or ICANS can be associated with CAR T-cell administration.

In some embodiments, beraprost, a beraprost isomer, or a pharmaceutically acceptable salt thereof does not interfere with cell killing mediated by the CAR T-cells. The cell killing can occur when CAR T-cells engage or interact with their corresponding antigen on a cancer which can result in the death of the cancer cells. In some embodiments, beraprost, a beraprost isomer, or a pharmaceutically acceptable salt thereof does not reduce or inhibit CAR T-cell mediated cell killing. In some embodiments, beraprost, a beraprost isomer, or a pharmaceutically acceptable salt thereof does not reduce CAR T-cell mediated killing by more than about 1%, about 2%, about 3%, about 4% about 5%, about 6%, about 7%, about 8%, about 9%, about 10%, about 0.01% to about 0.1%, about 0.1% to about 1%, about 1% to about 10%, about 10% to about 20%, about 0.5% to about 5%, about 5% to about 15%, about 15% to about 25% or more. In some embodiments, beraprost, a beraprost isomer, or a pharmaceutically acceptable salt thereof does not inhibit or reduce CAR T-cell activation or proliferation.

In some embodiments, beraprost, a beraprost isomer, or a pharmaceutically acceptable salt thereof can be used to treat grade 1 CRS, a grade 2 CRS, a grade 3 CRS or a grade 4 CRS. Treatment with beraprost, a beraprost isomer, or a pharmaceutically acceptable salt thereof can reduce the severity of CRS such that treatment can result in a higher grade CRS, for example, grade 4 CRS becoming a lower grade CRS, for example, grade 1. In some embodiments, the treatment with beraprost, a beraprost isomer, or a pharmaceutically acceptable salt thereof can eliminate or prevent CRS. In some embodiments, grade 1 CRS can include fever of about 38° C. or more. In some embodiments, grade 1 can include nausea, fatigue, headache and can require hospitalization. In some embodiments, grade 2 CRS can include fever of about 38° C. or more and hypotension not requiring vasopressors. Grade 2 CRS can further include hypoxia or decreased oxygen requiring low-flow nasal cannula or blow-by oxygen. In some embodiments, grade 2 CRS can include hypotension, and/or organ toxicity. In some embodiments, grade 3 can include fever of about 38° C. or more and hypotension requiring vasopressors with or without vasopressin treatment. Grade 3 can further include, hypoxia requiring high-flow nasal cannula, facemask, nonrebreather mask, or Venturi mask. In some embodiments, grade 4 can include fever of about 38° C. or more and hypotension requiring multiple vasopressors (excluding vasopressin) and/or organ toxicity. Grade 4 can further include, hypoxia requiring positive pressure (CPAP, BiPAP, intubation, mechanical ventilation) and/or organ toxicity.

In some embodiments, beraprost, a beraprost isomer, or a pharmaceutically acceptable salt thereof can be used to treat grade 1 ICANS, a grade 2 ICANS, a grade 3 ICANS or a grade 4 ICANS. Treatment with beraprost, a beraprost isomer, or a pharmaceutically acceptable salt thereof can reduce the severity of CRS such that treatment can result in a higher grade ICANS, for example, grade 4 ICANS becoming a lower grade ICANS, for example, grade 1 ICANS. In some embodiments, the treatment with beraprost, a beraprost isomer, or a pharmaceutically acceptable salt thereof can eliminate or prevent ICANS. In some embodiments, grade 1 ICANS can have an immune effector cell-associated encephalopathy (ICE) score: about 7-9. In some embodiments, grade 1 ICANS can include the following: consciousness: awakens spontaneously; seizure: none; motor findings: none; elevated ICP/cerebral edema: none. In some embodiments, grade 2 ICANS can include an ICE score of about 3-6. In some embodiments, grade 2 ICANS can include the following: consciousness: awakens to voice; seizure: none; motor findings: none; elevated ICP/cerebral edema: none. In some embodiments, grade 3 ICANS can include an ICE score of about 0-2. In some embodiments, grade 3 ICANS can include the following: consciousness: awakens only to tactile stimulus; seizure: any clinical seizure that resolves rapidly or nonconvulsive seizures on EEG that resolve with intervention; motor findings: none; elevated ICP/cerebral edema: focal/local edema on neuroimaging. In some embodiments, grade 4 ICANS can include an ICE score of about 0 (that is, patient or subject is unable to perform ICE). Grade 4 ICANS can include the following parameters: consciousness: subject is unarousable or requires vigorous or repetitive tactile stimuli to arouse, stupor or coma; seizure: life-threatening prolonged seizure (>5 min); or repetitive clinical or electrical seizures without return to baseline in between; motor findings: deep focal motor weakness such as hemiparesis or paraparesis; elevated ICP/cerebral edema: diffuse cerebral edema on neuroimaging; decerebrate or decorticate posturing; or cranial nerve VI palsy; or papilledema; or Cushing's triad.

In some embodiments, beraprost, a beraprost isomer, or a pharmaceutically acceptable salt thereof, can reduce the levels of one or more cytokines. Non-limiting examples of cytokines whose levels can be reduced, include, IL-1α, IL-1β, IL-2, IL-4, IL-6, IL-7, IL-8, IL-10, IL-12, IFN-γ, TNF-α, IP-10, MCP-1, MIP-1, RANTES, and/or GM-CSF. In some embodiments, the levels of one or more cytokines can be reduced by about 5%, about 10%, about 15%, about 20%, about 25%, about 30%, about 40%, about 45%, about 50%, about 55%, about 60%, about 65%, about 70%, about 75%, about 80%, about 85%, about 90%, about 95%, or more. In some embodiments, the levels of one or more cytokines can be reduced by about 1-10%, about 5-15%, about 10-20%, about 15-25%, about 20-30%, about 25-35%, about 30-40%, about 35-45%, about 40-50%, about 45-55%, about 50-60%, about 55-65%, about 60-70%, about 65-75%, about 70-80%, about 75-85%, about 80-90%, about 85-95%, or about 90-100%.

Beraprost, a beraprost isomer, or a pharmaceutically acceptable salt thereof can be used to reduce the levels of one or more cytokines that are known to increase from about 0-36 hours after CAR T-cell therapy. For example, beraprost, a beraprost isomer, or a pharmaceutically acceptable salt thereof can be used to reduce the levels of IL-2, IL-15 and/or MCP-1. In some embodiments, beraprost, a beraprost isomer, or a pharmaceutically acceptable salt thereof can be used to reduce the levels of one or more cytokines that are known to increase from about 2 days to 5 days after CAR T-cell therapy. For example, the cytokines can be IL-6, IL-8, IL-10, IFN-γ, or TNF-α.

In some methods, beraprost, a beraprost isomer, or a pharmaceutically acceptable salt thereof can be used in a combination therapy approach with other active pharmaceutical ingredients. For example, a method of treating cytokine release syndrome, ICANS, or both, associated with CAR T-cell administration in a subject can include administering CAR T-cells, a first pharmaceutical composition, and a second pharmaceutical composition to the subject, wherein: the first pharmaceutical composition comprises at least an effective amount of beraprost, a beraprost isomer, or a pharmaceutically acceptable salt thereof; and the second pharmaceutical composition comprises at least one corticosteroid, tocilizumab, IL-6 receptor blocker, or combinations thereof.

An additional method of treating cytokine release syndrome, ICANS, or both, associated with CAR T-cell administration can include administering CAR T-cells and a first pharmaceutical composition to the subject, wherein: the first pharmaceutical composition comprises at least an effective amount of beraprost, a beraprost isomer, or a pharmaceutically acceptable salt thereof; and the subject experiences reduced ICANS effects, Parkinsonism effects, or both, relative to a similar subject receiving administered CAR T-cells but not receiving the administered pharmaceutical composition. ICANS can be assessed and graded using a cognitive assessment tool called the “Immune Effector Cell-associated Encephalopathy (ICE) Assessment” tool, level of consciousness, presence and severity of seizures, motor control impairment, and presence of cerebral edema. Parkinsonism effects can be measured by various metrics including tremors, muscle stiffness, neurologic issues, psychomotor retardation, handwriting changes, and gait changes.

A further method of treating cytokine release syndrome, ICANS, or both, associated with CAR T-cell administration in a subject can include administering CAR T-cells and a first pharmaceutical composition to the subject, wherein: the first pharmaceutical composition comprises at least an effective amount of beraprost, a beraprost isomer, or a pharmaceutically acceptable salt thereof, and the subject experiences reduced severity measurements relative to a similar subject receiving administered CAR T-cells but not receiving the administered first pharmaceutical composition. Severity measurements can include event grades, event duration, event incidence, incidence of ICU or hospital stays, duration of ICU or hospital stays, onset timing, mortality, interference with antibiotics or other supportive medications, or combinations thereof. Additional severity measurements include use of supportive therapies, use of medical interventions, and use of intensive medical interventions such as intubation.

An additional method of treating cytokine release syndrome, ICANS, or both, associated with CAR T-cell administration in a subject can include administering CAR T-cells and a first pharmaceutical composition to the subject, wherein: the first pharmaceutical composition comprises at least an effective amount of beraprost, a beraprost isomer, or a pharmaceutically acceptable salt thereof, and the first pharmaceutical composition is administered once onset of CRS is detected by an increased level of one or more of cytokine MIF, IL-5, IL-17A, IL-23, IFN-γ, CXCL9/MIG, GCSF, VEGF-A, and TGF-β. Alternatively or additionally, onset of CRS can be detected by an increased level of one or more of cytokines such as, CCL2, IL-2, IL-6, IL-8, IL-10, IFN-γ, TNF-α, CXCL9, CXCL-10, VEGF, CCL3, GCSF, and GMCSF The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia.

An additional method of treating cytokine release syndrome, ICANS, or both, associated with CAR T-cell administration in a subject can include administering CAR T-cells and a first pharmaceutical composition to the subject, wherein: the first pharmaceutical composition comprises at least an effective amount of beraprost, a beraprost isomer, or a pharmaceutically acceptable salt thereof, the subject experiences CRS, ICANS, Parkinsonism effects, or a combination thereof, and the subject requires reduced treatment with at least one corticosteroid relative to a similar subject receiving administered CAR T-cells but not receiving the administered first pharmaceutical composition. In some examples, the subject does not require treatment with at least one corticosteroid.

Use of the described methods, kits, and pharmaceutical compositions can result in a reduction or elimination of CRS, ICANS, Parkinsonism, or combinations thereof. Reduction can be an improvement or resolution of undesirable physiological symptoms the patient subject is experiencing, a quantifiable reduction in one or more cytokine concentration, or both. The reduction can generally be reduced by any amount. For example, the reduction can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of disease, symptom, or other undesired property). Reduction can be relative to the effect observed with administration of the CAR T-cells but without administration of the first pharmaceutical composition.

CAR T-cells are typically delivered by infusion in one single administration, although multiple administrations are also possible. While CRS does not occur in all patients, about 50-100% of patients receiving CAR T-cell therapy develop CRS. CRS typically has an onset within the first week and can typically occur over the first two weeks post administration of CAR T-cells. The first pharmaceutical composition can be administered starting concurrently with the CAR T-cells (that is, no delay period), or starting after a delay period. In some examples, the first pharmaceutical composition can additionally be administered one or more times prior to administration of the CAR T-cells. The delay period can be a predetermined period of time after administration of the CAR T-cells (such as about 1 day, about 2 days, about 3 days, about 4 days, about 5 days, about 6 days, about 7 days, about 8 days, about 9 days, about 10 days, about 11 days, about 12 days, about 13 days, about 14 days, or more, or ranges between any two of these values). Example ranges of the delay period include about 3 days to 7 days, about 2 days to 5 days, about 3 days to 5 days, about 2 days to 5 days, about 4 days to 8 days, and so on. Alternatively, the delay period can last until onset of CRS is detected.

Various timings and sequences of administration of the first pharmaceutical composition are possible. For example, the first pharmaceutical composition can be administered prior to administration of the CAR T-cells, on the same day as administration of the CAR T-cells, after administration of the CAR T-cells, and combinations thereof. For example, the first pharmaceutical composition can be administered prior to administration of the CAR T-cells, on the same day as administration of the CAR T-cells, and after administration of the CAR T-cells. In one specific example, the first pharmaceutical composition can be administered one day prior to administration of the CAR T-cells, and then continues for at least about 14 additional days.

Onset of CRS can be detected by generally any method, such as detecting fever, headache, anorexia, nausea, fatigue, myalgia, hypoxia, low blood pressure (hypotension), impaired coagulation, capillary leakage, tachycardia, organ system failure and so on. For example, a simple method to detect onset of CRS is detecting fever. Alternatively, onset of CRS can be detected by monitoring increased levels of one or more cytokines such as IL-1α, IL-1β, IL-2, IL-4, IL-6, IL-7, IL-8, IL-10, IL-12, IFN-γ, TNF-α, IP-10, MCP-1, MIP-1, RANTES, and GM-CSF. Alternatively or additionally, onset of CRS can be detected by an increased level of one or more of cytokine CCL2, IL-2, IL-6, IL-8, IL-10, IFN-γ, TNF-α, CXCL9, CXCL-10, VEGF, CCL3, GCSF, and GMCSF. In some examples, onset of CRS can be detected by monitoring increased levels of one or more cytokines such as IL-6, IFN-γ, TNF-α, and IL-10. Alternatively or additionally, onset of CRS can be detected by monitoring increased levels of one or more cytokines such as MIF, IL-5, IL-17A, IL-23, IFN-γ, CXCL9/MIG, GCSF, VEGF-A, and TGF-β. The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia. In some embodiments, the first pharmaceutical composition can reduce the levels of one or more cytokines such as, but not limited to IL-1α, IL-1β, IL-2, IL-4, IL-6, IL-7, IL-8, IL-10, IL-12, IFN-γ, TNF-α, IP-10, MCP-1, MIP-1, RANTES, and GM-CSF.

For example, after the delay period or upon detecting onset of CRS, the first pharmaceutical composition can be administered for about 1 day, about 2 days, about 3 days, about 4 days, about 5 days, about 6 days, about 7 days, about 8 days, about 9 days, about 10 days, about 11 days, about 12 days, about 13 days, about 14 days, or longer, or ranges between any two of these values. CRS typically is resolved in about one week but has been documented to persist for about 30 days or beyond. In some examples, the first pharmaceutical composition can be administered for more than about 14 days, such as about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or longer, or ranges between any two of these values. Example ranges include about 1 day to 7 days, about 1 day to about 14 days, about 1 day to about 21 days, about 1 day to about 30 days, about 7 days to about 14 days, about 7 days to about 21 days, about 7 days to about 30 days, about 14 days to about 21 days, and about 21 days to about 30 days. In one specific example, the first pharmaceutical composition can be administered starting one day before administration of the CAR T-cells and continued for at least about 14 days.

The treatments can generally be performed at any effective schedule. For example, the first pharmaceutical compositions disclosed herein may be administered once, as needed, about once daily, about twice daily, about three times a day, about four times a day, about once a week, about twice a week, about three times a week, about four times a week, about five times a week, about six times a week, about seven times a week, about every other week, about every other day, or the like for one or more dosing cycles. It will be understood that the specific dose level and frequency of dosage for any particular subject can be varied and will depend upon a variety of factors including the species, age, body weight, general health, gender and diet of the subject, the mode and time of administration, rate of excretion, drug combination, and severity of the particular condition.

Administration may be performed by generally any method. Example delivery methods of administering include topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, and combinations thereof. In some examples, the administering comprises oral delivery, subcutaneous, inhalation, IV, or IM.

In some examples, administration of at least an effective amount of beraprost, a beraprost isomer, or a pharmaceutically acceptable salt thereof can reduce or eliminate the need for treatment with at least one corticosteroid, tocilizumab, IL-6 receptor blocker therapeutic, or combinations thereof, relative to a similar subject receiving administered CAR T-cells but not receiving the administered first pharmaceutical composition. In an ideal example, the subject does not require treatment with at least one corticosteroid, tocilizumab, or IL-6 receptor blocker therapeutic. In some examples, the subject does experience CRS, ICANS, Parkinsonism effects, or a combination thereof, but requires a reduced or eliminated dosage or administration of corticosteroid, tocilizumab, IL-6 receptor blocker therapeutic, or combinations thereof to achieve a similar (or superior) clinical effect as compared to a similar subject receiving administered CAR T-cells but not receiving the administered first pharmaceutical composition.

The CAR T-cells can generally be any CAR T-cells. Examples of current commercial CAR T-cell preparations include Abecma (idecabtagene vicleucel), Breyanzi (isocabtagene maraleucel), Kymriah (tisagenlecleucel), Tecartus (brexucabtagene autoleucel), Yescarta (axicabtagene ciloleucel), and Carvykti (ciltacabtagene autoleucel).

In some embodiments, beraprost, a beraprost isomer, or a pharmaceutically acceptable salt thereof can reduce or eliminate hospitalization associated with the development of CRS, ICANS or a combination thereof. In some embodiments, methods of the disclosure using beraprost, a beraprost isomer, or a pharmaceutically acceptable salt thereof can transform CAR-T therapy from primarily an in-patient to outpatient treatment.

Pharmaceutical Compositions

Pharmaceutical compositions described herein can be a first pharmaceutical composition or a second pharmaceutical composition. The first pharmaceutical composition can comprise at least one prostacyclin/prostaglandin analog. The second pharmaceutical composition can comprise at least one corticosteroid, tocilizumab, IL-6 receptor blocker, or combinations thereof.

Examples of prostacyclin or prostaglandin analogs include carbaprostacyclin, beraprost, taprostene, nileprost, iloprost, cicaprost, ciprostene, treprostinil, bonsentan, uoprost, eptaloprost, or an isomer thereof, and pharmaceutically acceptable salts thereof. In some embodiments, the first pharmaceutical composition comprises an effective amount of beraprost, a beraprost isomer, or a pharmaceutically acceptable salt thereof. Beraprost has a chemical formula C₂₄H₃₀O₅ and has a single carboxylic acid group. In some embodiments, the prostacyclin analog is a beraprost salt such as beraprost sodium (C₂₄H₂₉NaO₅; 2,3,3a,8b-tetrahydro-2-hydroxyl-1-(3-hydroxyl-4-methyl-1-octen-6-ynyl)-1H-cyclopenta[b]benzofuran-5-butanoic acid, sodium salt). Beraprost sodium (BPS; GP1001) can be a mixture of four isomers—two diastereomers (BPS-314 and BPS-315) and their two enantiomers each of which are BPS-314d (CTO1681; GP1681, (+)-[1R, 2R, 3aS, 8bS]-2,3,3a,8b-tetrahydro-2-hydroxy-1-[(E)-(3S,4S)-3-hydroxy-4-methyl-1-octen-6-ynyl)-1Hcyclopenta[b]benzofuran-5-butanoic acid, monosodium salt; also called esuberaprost sodium salt) and BPS-3141 (GP1684), and BPS-315d (GP1683) and BPS-315l (GP1682). Beraprost isomers are further described in U.S. Patent Publication No. US 2014-0275237 A1. In some examples, the first pharmaceutical composition can contain 1, 2, 3, or all 4 isomers of beraprost. In some embodiments, the beraprost isomer is BPS-314d (CTO1681; esuberaprost sodium salt).

Beraprost and methods for its preparation are shown in U.S. Pat. No. 7,345,181 and PCT Publication No. WO 2004/026224, entitled “Process for preparing prostaglandin derivatives and starting materials for the same”. Beraprost is commercially available from Yonsung Fine Chemicals (Gyeonggi-do, Republic of Korea). Beraprost, beraprost isomer, or pharmaceutically acceptable salt thereof can be present in the first pharmaceutical composition at generally any effective amount or effective concentration. Different pharmaceutical forms may have different amounts or concentrations of beraprost, a beraprost isomer, or pharmaceutically acceptable salt thereof.

In some embodiments, the first pharmaceutical composition can contain 1, 2, 3, or all 4 isomers of beraprost. Beraprost isomer refers to a beraprost molecule that has identical molecular formula to another beraprost molecule but has a distinct arrangement of their atoms in space. In some embodiments, an isomer of beraprost can be a structural isomer or a stereoisomer. In some embodiments, a structural isomer can comprise isomers in which bonds between the atoms differ but has the same molecular formula. In some embodiments, the isomer can be a stereoisomer of beraprost, wherein the bonds between the atoms are the same but the relative positions of the atoms differ. In some embodiments, a stereoisomer of beraprost can be a diastereomer of beraprost or an enantiomer of beraprost. The isomers can be different stereoisomers resulting from one or more chiral centers in their chemical structure. The different isomers of beraprost can have different biological activity, and sometimes can have no activity or even undesirable activity as compared to other desired isomers.

In some embodiments, the first pharmaceutical composition can comprise BPS-314d (CTO1681; GP1681, (+)-[1R, 2R, 3aS, 8bS]-2,3,3a,8b-tetrahydro-2-hydroxy-1-[(E)-(3S,4S)-3-hydroxy-4-methyl-1-octen-6-ynyl)-1Hcyclopenta[b]benzofuran-5-butanoic acid, monosodium salt; also called esuberaprost sodium salt). In some embodiments, the first pharmaceutical composition can comprise BPS-3141 (GP1684). In some embodiments, the first pharmaceutical composition can comprise BPS-315d (GP1683). In some embodiments, the first pharmaceutical composition can comprise BPS-315l (GP1682).

In some embodiments, the first pharmaceutical composition can include one or more enantiomers of beraprost. A common purity measurement is “enantiomeric excess” or “ee”. A racemic mixture has an ee of about 0%, while a completely pure enantiomer will have an ee value of about 100%. It is desirable an ee value of the first pharmaceutical composition of at least about 80%, at least about 85%, at least about 90%, at least about 91%, at least about 92%, at least about 93%, at least about 94%, at least about 95%, at least about 96%, at least about 97%, at least about 98%, at least about 99%, and ideally 100%.

As an example, a first pharmaceutical composition comprising beraprost isomer BPS-314d (CTO1681; GP1681; esuberaprost sodium salt) can have an ee value of at least about 80%, at least about 85%, at least about 90%, at least about 91%, at least about 92%, at least about 93%, at least about 94%, at least about 95%, at least about 96%, at least about 97%, at least about 98%, at least about 99%, and ideally 100%.

In some embodiments, one or more isomers of beraprost can be separated from the others such that only select isomers are included in the first pharmaceutical composition. In one embodiment, BPS-314d (CTO1681) can be separated from the other isomers in beraprost. Separation of the isomers from beraprost can be achieved using commercially-available chiral chromatography columns. Additional purification steps can be performed. In some embodiments, a single isomer can be obtained by chiral synthesis methods.

In some embodiments, synthetic methods can be used to prepare a desired isomer (for example, enantiomer or stereoisomer) in an enhanced concentration relative to undesired enantiomers or stereoisomers.

In some embodiments, the first pharmaceutical compositions can have a high purity both at the time of manufacture as well as at later times such as at time of use. In some embodiments, the first pharmaceutical composition can have a low or no level of degradation products of beraprost, a beraprost isomer, or a pharmaceutically acceptable salt thereof.

Impurities are undesired in the first pharmaceutical compositions and can come from different sources. An impurity can be a component of the raw materials, residual solvents, or synthesis that was incompletely removed or purified from the final desired product. A contaminant can be a substance that is unintentionally included with the final desired product due to the manufacturing environment or other sources. Impurities and contaminants can be harmful or harmless and can be identified or unidentified. In some examples, the first a pharmaceutical composition contains not more than about 0.1 wt. % impurity or contaminant, not more than about 0.2 wt. % impurity or contaminant, or not more than about 0.5 wt. % impurity or contaminant. In an ideal example, the composition does not contain detectable impurities or contaminants.

One or more degradation products can arise from various paths such as instability, degradation, or oxidation of the first pharmaceutical composition itself, or by incompatibility or reaction of the first pharmaceutical composition with another component of the composition (such as one or more excipients), moisture, or the composition packaging. In some examples, the first pharmaceutical composition contains not more than about 0.1 wt. % degradation product, not more than about 0.2 wt. % degradation product or not more than about 0.5 wt. % degradation product. In one example, the composition does not contain detectable degradation products.

The daily dose (mass) of prostacyclin/prostaglandin analog or beraprost, a beraprost isomer, or pharmaceutically acceptable salt thereof can generally be any effective amount or dosage. For example, the therapeutically effective amount (in micrograms) may include about 0.1 μg to about 100 μg, about 10 μg to about 90 μg, or about 15 μg to about 90 μg, or about 0.1 μg to about 5000 μg. The mass values are the combined salt weight, that is the anion and cation together. Specific examples of therapeutically effective amounts include about 0.1 μg, about 1 μg, about 5 μg, about 10 μg, about 15 μg, about 20 μg, about 30 μg, about 40 μg, about 45 μg, about 50 μg, about 60 μg, about 70 μg, about 80 μg, about 90 μg, about 100 μg, and ranges between any two of these values. When administered in two or more daily doses, the amount in each dose can be added together to yield a total daily dose. For example, CTO1681 may be administered at a dose of about 15-90 μg/day divided into about 3 doses, and each individual dose of about 5-30 ag.

In some embodiments, the effective amount of beraprost, a beraprost isomer, or a pharmaceutically acceptable salt thereof are present in a unit dose (mass) of the first pharmaceutical composition is at least about 1 microgram, about 1 microgram to about 100 micrograms, about 1 microgram to about 80 micrograms, about 1 microgram to about 60 micrograms, about 1 microgram to about 50 micrograms, about 1 microgram to about 40 micrograms, about 51 microgram to about 30 micrograms, about 1 microgram to about 20 micrograms, about 1 mg to about 10 micrograms, or about 1 microgram to about 5 micrograms, or any value between these ranges. Specific examples include about 1 microgram, about 5 micrograms, about 10 micrograms, about 25 micrograms, about 50 micrograms, about 75 micrograms, about 100 micrograms, about 60 micrograms to about 360 micrograms, or ranges between any two of these values.

In some embodiments, the amount of beraprost, a beraprost isomer, or a pharmaceutically acceptable salt thereof can be calculated based on the presence of a single desired isomer. For example, if a single isomer, such as BPS-314d (CTO1681; also esuberaprost sodium salt) is desired at an amount of about 15 micrograms to about 90 micrograms, this is equivalent to an amount of about 60 micrograms to about 360 micrograms of a racemic mixture of four isomers (where the amount of a single isomer is one-quarter of the mass).

In some embodiments, the first pharmaceutical composition comprising beraprost, a beraprost isomer, or a pharmaceutically acceptable salt thereof achieves a Cmax of about 0.01 nanomolar to about 10 nanomolar, about 0.01 nanomolar to about 5 nanomolar, about 0.01 nanomolar to about 3 nanomolar, about 0.01 nanomolar to about 2 nanomolar, about 0.01 nanomolar to about 1 nanomolar, about 0.01 nanomolar to about 0.5 nanomolar, or any values between these ranges. Specific examples include about 0.01 nanomolar, about 0.05 nanomolar, about 0.075 nanomolar, about 0.1 nanomolar, about 0.5 nanomolar, about 1 nanomolar, about 2 nanomolar, about 5 nanomolar, or about 10 nanomolar.

In some embodiments, the first pharmaceutical composition comprising beraprost, a beraprost isomer, or a pharmaceutically acceptable salt thereof achieves a Tₘₐₓ at about 0.1 hour to about 5 hours, about 0.1 hour to about 4 hours, about 0.1 hour to about 3 hours, about 0.1 hour to about 2 hours, about 0.1 hour to about 1 hours, or any specific value between these ranges. Specific examples include about 0.1 hour, about 0.5 hour, about 1 hour, about 1.5 hours, about 1.7 hours, about 2 hours, or about 5 hours.

In some embodiments, the first pharmaceutical composition comprising beraprost, a beraprost isomer, or a pharmaceutically acceptable salt thereof achieves an AUC of about 0.01 ng·hr/mL to about 30 ng·hr/mL over a 48 hour period, about 0.01 ng·hr/mL to about 20 ng·hr/mL over a 48 hour period, about 0.01 ng·hr/mL to about 10 ng·hr/mL over a 48 hour period, about 0.01 ng·hr/mL to about 5 ng·hr/mL over a 48 hour period, about 0.01 ng·hr/mL to about 3 ng·hr/mL over a 48 hour period, about 0.01 ng·hr/mL to about 2 ng·hr/mL over a 48 hour period, or about 0.01 ng·hr/mL to about 1 ng·hr/mL over a 48 hour period. Specific examples include about 0.01 ng·hr/mL, about 0.05 ng·hr/mL, about 0.1 ng·hr/mL, about 0.5 ng·hr/mL, about 1 ng·hr/mL, about 2 ng·hr/mL, about 5 ng·hr/mL, about 10 ng·hr/mL, or about 30 ng·hr/mL.

In some examples, the first pharmaceutical composition can further comprise at least one anti-inflammatory component such as at least one corticosteroid or at least one therapeutic monoclonal antibody.

In some examples, the first pharmaceutical composition further comprises one or more pharmaceutically acceptable excipients. Examples of pharmaceutically acceptable excipients that may be present in the composition include but not limited to fillers/vehicles, solvents/co-solvents, preservatives, antioxidants, suspending agents, surfactants, antifoaming agents, buffering agents, chelating agents, sweeteners, flavoring agents, binders, extenders, disintegrants, diluents, lubricants, fillers, wetting agents, glidants, and combinations thereof.

In some examples, the first pharmaceutical composition can further comprise one or more exemplary fillers. Examples of exemplary fillers include cellulose and cellulose derivatives such as microcrystalline cellulose; starches such as dry starch, hydrolyzed starch, and starch derivatives such as corn starch; cyclodextrin; sugars such as powdered sugar and sugar alcohols such as lactose, mannitol, sucrose and sorbitol; inorganic fillers such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, or combinations thereof. Fillers may be present in the composition from about 20 wt % to about 65 wt %, about 20 wt % to about 50 wt %, about 20 wt % to about 40 wt %, about 45 wt % to about 65 wt %, about 50 wt % to about 65 wt %, or about 55 wt % to about 65 wt % of the total weight of the composition, or any value between these ranges.

In some examples, the first pharmaceutical composition further comprises one or more disintegrants. Examples of disintegrants include starches, alginic acid, crosslinked polymers such as crosslinked polyvinylpyrrolidone, croscarmellose sodium, potassium starch glycolate, sodium starch glycolate, clays, celluloses, starches, gums, or combinations thereof. Disintegrants may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some examples, the first pharmaceutical composition further comprises one or more binders, including but not limited to celluloses such as hydroxypropylcellulose, methyl cellulose, and hydroxypropylmethylcellulose; starches such as corn starch, pregelatinized starch, and hydroxpropyl starch; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof. Binders may be present in the composition from about 0.5 wt % to about 5 wt %, about 0.5 wt % to about 4 wt %, about 0.5 wt % to about 3 wt %, about 0.5 wt % to about 2 wt %, or about 0.5 wt % to about 1 wt % of the total weight of the composition, or any value between these ranges.

In some examples, the first pharmaceutical composition further comprises one or more wetting agents, including but not limited to oleic acid, glyceryl monostearate, sorbitan mono-oleate, sorbitan monolaurate, triethanolamine oleate, polyoxyethylene sorbitan mono-oleate, polyoxyethylene sorbitan monolaurate, sodium oleate, sodium lauryl sulfate, poloxamers, poloxamer 188, polyoxyethylene ethers, polyoxyethylene sorbitan fatty acid esters, polyoxyethylene fatty acid esters, polyethylene glycol fatty acid esters, polyoxyethylene hardened castor oil, polyoxyethylene alkyl ethers, polysorbates, cetyl alcohol, glycerol fatty acid esters (for example, triacetin, glycerol monostearate, etc.), polyoxymethylene stearate, sodium lauryl sulfate, sorbitan fatty acid esters, sucrose fatty acid esters, benzalkonium chloride, polyethoxylated castor oil, and combinations thereof. Wetting agents may be present in the composition from about 0.1 wt % to about 1 wt %, about 0.1 wt % to about 2 wt %, about 0.1 wt % to about 3 wt %, about 0.1 wt % to about 4 wt %, or about 0.1 wt % to about 5 wt % of the total weight of the composition, or any value between these ranges.

In some examples, the first pharmaceutical composition further comprises one or more lubricants, including but not limited to stearic acid, magnesium stearate, calcium hydroxide, talc, corn starch, sodium stearyl fumarate, alkali-metal and alkaline earth metal salts, waxes, boric acid, sodium benzoate, sodium acetate, sodium chloride, leucine, polyethylene glycol (PEG), a methoxypolyethylene glycol, propylene glycol, sodium oleate, glyceryl behenate, glyceryl palmitostearate, glyceryl benzoate, magnesium lauryl sulfate, sodium lauryl sulfate, and combinations thereof. Lubricants may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some examples, the first pharmaceutical composition further comprises one or more glidants, including but not limited to colloidal silicon dioxide, talc, sodium lauryl sulfate, native starch, and combinations thereof. Glidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some examples, the first pharmaceutical composition is a tablet and further comprises a top coat, such as hydroxypropyl-methylcellulose coating or polyvinyl alcohol coating, and are available under the trade name Opadry, such as Opadry White, Opadry II (Opadry is a registered trademark of BPSI Holdings LLC, Wilmington, DE, USA). Top coats may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some examples, the first pharmaceutical composition can further comprise one or more preservative agents. Examples of preservative agents include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, or combinations thereof. Preservative agents can be included in the liquid dosage form. The preservative agents can be in an amount sufficient to extend the shelf-life or storage stability, or both, of the liquid dosage form. Preservatives may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some examples, the first pharmaceutical composition can further comprise one or more flavoring agents. Examples of flavoring agents include synthetic flavor oils and flavoring aromatics and/or natural oils, extracts from plants leaves, flowers, fruits, and so forth and the like or any combinations thereof. Additional examples include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, strawberry flavor, tutti-fruity flavor, mint flavor, or any combinations thereof. Flavoring agents may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some examples, the first pharmaceutical composition can further comprise one or more antioxidants. Examples of antioxidants include flavonoids, anthocyanidins, anthocyanins, proanthocyanidins, or combinations thereof. Antioxidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

Physical Form of the Pharmaceutical Composition

The first and/or second pharmaceutical compositions can generally be in any physical form suitable for use in treating a subject. These forms can be referred to as a unit dosage form, such as an individual pill or tablet. In some examples, the first and/or second pharmaceutical compositions can be formulated as tablets, capsules, granules, powders, liquids, suspensions, gels, syrups, slurries, suppositories, patches, nasal sprays, aerosols, injectables, implantable sustained-release formulations, or mucoadherent films. In some examples, the first and/or second pharmaceutical composition may be formed as a tablet, a bi-layer tablet, a capsule, a multiparticulate, a drug coated sphere, a matrix tablet, or a multicore tablet. A physical form can be selected according to the desired method of treatment. In some examples, the physical form can be a liquid, for example for oral or IV, IP, IM, or IT administration.

The first and/or second pharmaceutical compositions can be manufactured by various conventional methods such as conventional mixing, dissolving, granulating, dragee-making, levigating, emulsifying, encapsulating, entrapping, or lyophilizing processes. The first and/or second pharmaceutical compositions can be formulated in a conventional manner using one or more physiologically acceptable carriers, diluents, excipients, or auxiliaries that facilitate processing of the active agent into preparations that can be used pharmaceutically. Proper formulation can be selected upon the route of administration chosen.

For topical administration the first and/or second pharmaceutical compositions described herein may be formulated as solutions, gels, ointments, creams, suspensions, and the like as are well-known in the art. Systemic compositions include, but are not limited to, those designed for administration by injection, for example, subcutaneous, intravenous injection (IV), intramuscular injection (IM), intrathecal injection (IT), intraperitoneal injection (IP), as well as those designed for transdermal, subcutaneous, transmucosal oral, or pulmonary administration. For injection, the first and/or second pharmaceutical compositions can be formulated in aqueous solutions, preferably in physiologically compatible buffers such as Hanks solution, Ringer's solution, or physiological saline buffer and/or in certain emulsion formulations. The solution can contain one or more formulatory agents such as suspending, stabilizing and/or dispersing agents. In certain examples the first and/or second pharmaceutical compositions can be provided in powder form for constitution with a suitable vehicle, for example, sterile pyrogen-free water, before use. For transmucosal administration, one or more penetrants appropriate to the barrier to be permeated can be used in the formulation. Such penetrants are generally known in the art.

For oral administration, the first and/or second pharmaceutical compositions can combine the beraprost, an isomer or pharmaceutically acceptable salt thereof with one or more pharmaceutically acceptable carriers well known in the art. Such carriers facilitate formulation as tablets, pills, dragees, capsules, liquids, gels, syrups, slurries, suspensions, and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients, or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the first and/or second pharmaceutical compositions can be delivered in the form of an aerosol spray from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

In some examples, the first and/or second pharmaceutical compositions are immediate release first and/or second pharmaceutical compositions, modified release first and/or second pharmaceutical compositions, or a combination thereof. In some examples, the immediate release first pharmaceutical composition releases the beraprost, a beraprost isomer, or a pharmaceutically acceptable salt thereof within a short period of time after administration, typically less than about 4 hours, less than about 3.5 hours, less than about 3 hours, less than about 2.5 hours, less than about 2 hours, less than about 90 minutes, less than about 60 minutes, less than about 45 minutes, less than about 30 minutes, less than about 20 minutes, or less than about 10 minutes.

In some embodiments, the first pharmaceutical composition is an immediate release first pharmaceutical composition comprising microcrystalline cellulose, hydroxypropyl cellulose, lactose monohydrate, pregelatinized starch, magnesium stearate, and/or purified water. In some embodiments, the composition can include a coating of prepared using Opadry® film coating process.

In some examples, the modified release composition may release the beraprost, a beraprost isomer, or a pharmaceutically acceptable salt thereof at a sustained or controlled rate over an extended period of time or may release it after a lag time after administration. For example, it may be released from the composition 4 hours after administration, 8 hours after administration, 12 hours after administration, 16 hours after administration, or 24 hours after administration. Modified release compositions include extended release, sustained release, and delayed release compositions. In some examples, the modified release compositions may release about 10% in about 2 hours, about 20% in 2 hours, about 40% in about 2 hours, about 50% in about 2 hours, about 10% in about 3 hours, about 20% in 3 hours, about 40% in about 3 hours, about 50% in about 3 hours, about 10% in about 4 hours, about 20% in 4 hours, about 40% in about 4 hours, about 50% in about 4 hours, about 10% in about 6 hours, about 20% in 6 hours, about 40% in about 6 hours, or about 50% in about 6 hours.

In some examples, modified release compositions may comprise a matrix selected from microcrystalline cellulose, sodium carboxymethylcellulose, hydroxyalkylcelluloses such as hydroxy propyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinyl acetate phthalate, polyalkylmethacrylates, polyvinyl acetate and mixtures thereof.

The modified release compositions can also be formulated as a depot preparation. Such long-acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Subjects to be Treated

The subject can generally be any mammal. Examples of subjects include a non-human primate, a human, a dog, a cat, a mouse, a rat, a cow, a goat, a sheep, a rabbit, a horse, a monkey, and a pig. In some examples, the subject is a human. The terms “subject,” “individual” or “patient” are used interchangeably and as used herein are intended to include human and non-human animals. Non-human animals include all vertebrates, for example, mammals and non-mammals, such as non-human primates, monkeys, sheep, dogs, rats, cats, cows, horses, chickens, amphibians, and reptiles. Examples of mammals include non-human primates, monkeys, sheep, dogs, cats, cows, and horses. In some examples, the subject is a human or humans. The methods are suitable for treating humans having cancer. The subject may be symptomatic or asymptomatic.

Kits

In additional examples, kits are provided for treating cytokine release syndrome, ICANS, or both, associated with CAR T-cell administration in a subject. The kits can comprise a first container containing a first pharmaceutical composition comprising at least an effective amount of prostacyclin/prostaglandin analog or beraprost, a beraprost isomer, or pharmaceutically acceptable salt thereof, a second container containing a second pharmaceutical composition comprising at least one corticosteroid, tocilizumab, IL-6 receptor blocker, or combinations thereof; a third container containing CAR T-cells; and instructions for the administration of the first pharmaceutical composition, the second pharmaceutical composition, and the CAR T-cells to the subject. Any of the above-described pharmaceutical compositions can be included in the kit. The kit can further comprise a fourth container, and so on containing additional pharmaceutical compositions or other active ingredients. In some examples, the first container can contain a first pharmaceutical composition, a second container containing a second pharmaceutical composition, a third container containing CAR T-cells, and a fourth container can contain at least one solvent or solvents to be mixed with the first pharmaceutical composition before administering to the subject according to the instructions. In an example, the kit can comprise a first container containing beraprost, a beraprost isomer, or a pharmaceutically acceptable salt thereof, a second container containing a second pharmaceutical composition comprising at least one corticosteroid, tocilizumab, IL-6 receptor blocker, or combinations thereof, a third container containing CAR T-cells, and a fourth container containing an aqueous solvent. In some examples, the beraprost isomer is BPS-314d (CTO1681).

In other examples, the kit can comprise: a first container containing a first pharmaceutical composition comprising at least an effective amount of beraprost, a beraprost isomer, or a pharmaceutically acceptable salt thereof; a second container containing a second pharmaceutical composition comprising at least one corticosteroid, tocilizumab, IL-6 receptor blocker, or combinations thereof, and instructions for the administration of the first pharmaceutical composition, the second pharmaceutical composition, and CAR T-cells to a subject. The kit can further contain a third container containing CAR T-cells. The kit can further comprise a fourth container containing water or an aqueous solution.

Further aspects of the invention are also described in the following numbered clauses:

1. A method of treating cytokine release syndrome, ICANS, or both, associated with CAR T-cell administration in a subject, the method comprising administering CAR T-cells, a first pharmaceutical composition, and a second pharmaceutical composition to the subject, wherein:
the first pharmaceutical composition comprises at least an effective amount of beraprost or a pharmaceutically acceptable salt thereof, and the second pharmaceutical composition comprises at least one corticosteroid, tocilizumab, IL-6 receptor blocker, or combinations thereof.

2. The method of clause 1, wherein the pharmaceutically acceptable salt is beraprost sodium salt.

3. The method of clause 1, wherein the beraprost comprises at least one of BPS-314d, BPS-3141, BPS-315d, and BPS-315l.

4. The method of clause 1, wherein the beraprost is BPS-314d (esuberaprost sodium salt).

5. The method of clause 1, wherein:
the CAR T-cell administration is performed to treat cancer; and
   the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma.

6. The method of clause 1, wherein
the CAR T-cell administration is performed to treat cancer; and
the cancer is brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

7. The method of clause 1,
the CAR T-cell administration is performed to treat cancer; and
wherein the cancer is a B-cell lymphoma.

8. The method of clause 1, wherein the subject is a mammal.

9. The method of clause 1, wherein the subject is a non-human primate, monkey, cat, dog, pig, cow, goat, horse, sheep, or rabbit.

10. The method of clause 1, wherein the subject is a human.

11. The method of clause 1, wherein:
the first pharmaceutical composition is administered to the subject before the CAR T-cells are administered to the subject; and
the second pharmaceutical composition is administered to the subject after the CAR T-cells are administered to the subject.

12. The method of clause 1, wherein:
the first pharmaceutical composition is administered to the subject before and after the CAR T-cells are administered to the subject; and
the second pharmaceutical composition is administered to the subject after the CAR T-cells are administered to the subject.

13. The method of clause 1, wherein the CAR T-cells, the first pharmaceutical composition, and the second pharmaceutical composition are administered to the subject concurrently.

14. The method of clause 1, wherein the first pharmaceutical composition and the second pharmaceutical composition are administered to the subject after the CAR T-cells are administered to the subject.

15. The method of clause 1, wherein:
the first pharmaceutical composition and the second pharmaceutical composition are administered to the subject after the CAR T-cells are administered to the subject; and
the first pharmaceutical composition and the second pharmaceutical composition are administered concurrently.

16. The method of clause 1, wherein the first pharmaceutical composition and the second pharmaceutical composition are administered to the subject before the CAR T-cells are administered to the subject.

17. The method of clause 1, wherein the first pharmaceutical composition is administered to the subject starting about 1 day, about 2 days, about 3 days, about 4 days, about 5 days, about 6 days, or about 7 days after the CAR T-cells are administered to the subject.

18. The method of clause 1, wherein the first pharmaceutical composition is administered to the subject starting about 3 days to about 7 days after the CAR T-cells are administered to the subject.

19. The method of clause 1, wherein the first pharmaceutical composition is administered to the subject:
after the CAR T-cells are administered to the subject; and once onset of CRS is detected.

20. The method of clause 1, wherein the first pharmaceutical composition is administered to the subject:
after the CAR T-cells are administered to the subject; and
once onset of CRS is detected by an increased level of one or more of cytokine IL-1α, IL-β, IL-2, IL-4, IL-6, IL-7, IL-8, IL-10, IL-12, IFN-γ, TNF-α, IP-10, MCP-1, MIP-1, RANTES, and GM-CSF.

21. The method of clause 1, wherein the first pharmaceutical composition is administered to the subject:
after the CAR T-cells are administered to the subject; and once onset of CRS is detected by an increased level of one or more of cytokine IL-6, IL-10, IFN-γ, and TNF-α.

22. The method of clause 1, wherein the first pharmaceutical composition is administered for a period of about 1 day to about 30 days.

23. The method of clause 1, wherein the first pharmaceutical composition is administered for a period of about 7 days to about 14 days.

24. The method of clause 1, wherein the first pharmaceutical composition is administered starting one day before administration of the CAR T-cells and continued for a period of at least about 14 days.

25. The method of clause 1, further comprising administering the first pharmaceutical composition to the subject before administration of the CAR T-cells.

26. The method of clause 1, wherein the subject experiences reduced CRS relative to a similar subject receiving administered CAR T-cells but not receiving the administered first pharmaceutical composition.

27. The method of clause 1, wherein the subject does not experience CRS.

28. The method of clause 1, wherein the CAR T-cells are autologous.

29. The method of clause 1, wherein the CAR T-cells are allogenic.

30. The method of clause 1, wherein the first pharmaceutical composition further comprises at least one excipient, at least one filler, at least one disintegrant, at least one binder, at least one wetting agent, at least one lubricant, at least one glidant, at least one preservative agent, at least one flavoring agent, at least one antioxidant, or combinations thereof.

31. The method of clause 1, wherein the first pharmaceutical composition is formulated as a tablet, capsule, granule, powder, liquid, suspension, gel, syrup, slurry, suppository, patch, nasal spray, aerosol, injectable, implantable sustained-release formulation, or mucoadherent film.

32. The method of clause 1, wherein the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

33. The method of clause 1, wherein the administering comprises oral delivery or intravenous injection (IV) delivery.

34. The method of clause 1, wherein the beraprost or pharmaceutically acceptable salt thereof is present in a unit dose of the first pharmaceutical composition in an amount of about 1 microgram to about 100 micrograms.

35. The method of clause 1, wherein the administering comprises delivering the first pharmaceutical composition to the subject at an amount of beraprost or a pharmaceutically acceptable salt thereof at least about 0.1 microgram.

36. The method of clause 1, wherein the administering comprises delivering the first pharmaceutical composition to the subject at an amount of beraprost or a pharmaceutically acceptable salt thereof at about 0.1 microgram to about 5000 micrograms.

37. The method of clause 1, wherein the administering comprises delivering the first pharmaceutical composition to the subject at an amount of beraprost or a pharmaceutically acceptable salt thereof at about 15 micrograms to about 90 micrograms.

38. The method of clause 1, wherein the administering comprises delivering the first pharmaceutical composition to the subject at an amount of beraprost or a pharmaceutically acceptable salt thereof at about 60 micrograms to about 360 micrograms.

39. The method of clause 1, wherein the CAR T-cells are Abecma (idecabtagene vicleucel), Breyanzi (isocabtagene maraleucel), Kymriah (tisagenlecleucel), Tecartus (brexucabtagene autoleucel), Yescarta (axicabtagene ciloleucel), Carvykti (ciltacabtagene autoleucel), Blincyto (blinatumomab), or combinations thereof.

40. A method of treating cytokine release syndrome, ICANS, or both, associated with CAR T-cell administration in a subject, the method comprising administering CAR T-cells and a first pharmaceutical composition to the subject, wherein:
the first pharmaceutical composition comprises at least an effective amount of beraprost or a pharmaceutically acceptable salt thereof, and
the subject experiences reduced ICANS effects, Parkinsonism effects, or both, relative to a similar subject receiving administered CAR T-cells but not receiving the administered first pharmaceutical composition.

41. The method of clause 40, wherein the pharmaceutically acceptable salt is beraprost sodium salt.

42. The method of clause 40, wherein the beraprost comprises at least one of BPS-314d, BPS-3141, BPS-315d, and BPS-315l.

43. The method of clause 40, wherein the beraprost is BPS-314d (esuberaprost sodium salt).

44. The method of clause 40, wherein:
the CAR T-cell administration is performed to treat cancer; and
the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma.

45. The method of clause 40, wherein:
the CAR T-cell administration is performed to treat cancer; and
the cancer is brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

46. The method of clause 40, wherein:
the CAR T-cell administration is performed to treat cancer; and
the cancer is a B-cell lymphoma.

47. The method of clause 40, wherein the subject is a mammal.

48. The method of clause 40, wherein the subject is a non-human primate, monkey, cat, dog, pig, cow, goat, horse, sheep, or rabbit.

49. The method of clause 40, wherein the subject is a human.

50. The method of clause 40, wherein the first pharmaceutical composition is administered to the subject before the CAR T-cells are administered to the subject.

51. The method of clause 40, wherein:
the first pharmaceutical composition is administered to the subject before the CAR T-cells are administered; and
the first pharmaceutical composition is administered to the subject after the CAR T-cells are administered to the subject.

52. The method of clause 40, wherein the CAR T-cells and the first pharmaceutical composition are administered to the subject concurrently.

53. The method of clause 40, wherein the first pharmaceutical composition is administered to the subject after the CAR T-cells are administered to the subject.

54. The method of clause 40, wherein the first pharmaceutical composition is administered to the subject starting about 1 day, about 2 days, about 3 days, about 4 days, about 5 days, about 6 days, or about 7 days after the CAR T-cells are administered to the subject.

55. The method of clause 40, wherein the first pharmaceutical composition is administered to the subject starting about 3 days to about 7 days after the CAR T-cells are administered to the subject.

56. The method of clause 40, wherein the first pharmaceutical composition is administered to the subject:
after the CAR T-cells are administered to the subject; and
once onset of CRS is detected.

57. The method of clause 40, wherein the first pharmaceutical composition is administered to the subject:
after the CAR T-cells are administered to the subject; and
once onset of CRS is detected by an increased level of one or more of cytokine IL-1α, IL-β, IL-2, IL-4, IL-6, IL-7, IL-8, IL-10, IL-12, IFN-γ, TNF-α, IP-10, MCP-1, MIP-1, RANTES, and GM-CSF.

58. The method of clause 40, wherein the first pharmaceutical composition is administered to the subject:
after the CAR T-cells are administered to the subject; and
once onset of CRS is detected by an increased level of one or more of inflammatory markers associated with CRS CRP (C-reactive protein) and ferritin.

59. The method of clause 40, wherein the first pharmaceutical composition is administered for a period of about 1 day to about 30 days.

60. The method of clause 40, wherein the first pharmaceutical composition is administered for a period of about 7 days to about 14 days.

61. The method of clause 40, wherein the first pharmaceutical composition is administered starting one day before administration of the CAR T-cells and continued for a period of at least about 14 days.

62. The method of clause 40, further comprising administering the first pharmaceutical composition to the subject before administration of the CAR T-cells.

63. The method of clause 40, wherein the subject experiences reduced ICANS effects relative to a similar subject receiving administered CAR T-cells but not receiving the administered first pharmaceutical composition.

64. The method of clause 40, wherein the subject does not experience ICANS effects.

65. The method of clause 40, wherein the subject experiences reduced Parkinsonism effects relative to a similar subject receiving administered CAR T-cells but not receiving the administered first pharmaceutical composition.

66. The method of clause 40, wherein the subject does not experience Parkinsonism effects.

67. The method of clause 40, wherein the CAR T-cells are autologous.

68. The method of clause 40, wherein the CAR T-cells are allogenic.

69. The method of clause 40, wherein the first pharmaceutical composition further comprises at least one excipient, at least one filler, at least one disintegrant, at least one binder, at least one wetting agent, at least one lubricant, at least one glidant, at least one preservative agent, at least one flavoring agent, at least one antioxidant, or combinations thereof.

70. The method of clause 40, wherein the first pharmaceutical composition is formulated as a tablet, capsule, granule, powder, liquid, suspension, gel, syrup, slurry, suppository, patch, nasal spray, aerosol, injectable, implantable sustained-release formulation, or mucoadherent film.

71. The method of clause 40, wherein the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

72. The method of clause 40, wherein the administering comprises oral delivery or intravenous injection (IV) delivery.

73. The method of clause 40, wherein the beraprost or pharmaceutically acceptable salt thereof is present in a unit dose of the first pharmaceutical composition in an amount of about 1 microgram to about 100 micrograms.

74. The method of clause 40, wherein the administering comprises delivering the first pharmaceutical composition to the subject at an amount of beraprost or a pharmaceutically acceptable salt thereof at least about 0.1 microgram.

75. The method of clause 40, wherein the administering comprises delivering the first pharmaceutical composition to the subject at an amount of beraprost or a pharmaceutically acceptable salt thereof at about 0.1 microgram to about 5000 micrograms.

76. The method of clause 40, wherein the administering comprises delivering the first pharmaceutical composition to the subject at an amount of beraprost or a pharmaceutically acceptable salt thereof at about 15 micrograms to about 90 micrograms.

77. The method of clause 40, wherein the administering comprises delivering the first pharmaceutical composition to the subject at an amount of beraprost or a pharmaceutically acceptable salt thereof at about 60 micrograms to about 360 micrograms.

78. The method of clause 40, wherein the CAR T-cells are Abecma (idecabtagene vicleucel), Breyanzi (isocabtagene maraleucel), Kymriah (tisagenlecleucel), Tecartus (brexucabtagene autoleucel), Yescarta (axicabtagene ciloleucel), Carvykti (ciltacabtagene autoleucel), Blincyto (blinatumomab), or combinations thereof.

79. A method of treating cytokine release syndrome, ICANS, or both, associated with CAR T-cell administration in a subject, the method comprising administering CAR T-cells and a first pharmaceutical composition to the subject, wherein:
the first pharmaceutical composition comprises at least an effective amount of beraprost or a pharmaceutically acceptable salt thereof, and
the subject experiences reduced severity measurements relative to a similar subject receiving administered CAR T-cells but not receiving the administered first pharmaceutical composition.

80. The method of clause 79, wherein the pharmaceutically acceptable salt is beraprost sodium salt.

81. The method of clause 79, wherein the beraprost comprises at least one of BPS-314d, BPS-3141, BPS-315d, and BPS-315l.

82. The method of clause 79, wherein the beraprost is BPS-314d (esuberaprost sodium salt).

83. The method of clause 79, wherein:
the CAR T-cell administration is performed to treat cancer; and
the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma.

84. The method of clause 79, wherein:
the CAR T-cell administration is performed to treat cancer; and
the cancer is brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

85. The method of clause 79, wherein:
the CAR T-cell administration is performed to treat cancer; and
the cancer is a B-cell lymphoma.

86. The method of clause 79, wherein the subject is a mammal.

87. The method of clause 79, wherein the subject is a non-human primate, monkey, cat, dog, pig, cow, goat, horse, sheep, or rabbit.

88. The method of clause 79, wherein the subject is a human.

89. The method of clause 79, wherein the first pharmaceutical composition is administered to the subject before the CAR T-cells are administered to the subject.

90. The method of clause 79, wherein:
the first pharmaceutical composition is administered to the subject before the CAR T-cells are administered; and
the first pharmaceutical composition is administered to the subject after the CAR T-cells are administered to the subject.

91. The method of clause 79, wherein the CAR T-cells and the first pharmaceutical composition are administered to the subject concurrently.

92. The method of clause 79, wherein the first pharmaceutical composition is administered to the subject after the CAR T-cells are administered to the subject.

93. The method of clause 79, wherein the first pharmaceutical composition is administered to the subject starting about 1 day, about 2 days, about 3 days, about 4 days, about 5 days, about 6 days, or about 7 days after the CAR T-cells are administered to the subject.

94. The method of clause 79, wherein the first pharmaceutical composition is administered to the subject starting about 3 days to about 7 days after the CAR T-cells are administered to the subject.

95. The method of clause 79, wherein the first pharmaceutical composition is administered to the subject:
after the CAR T-cells are administered to the subject; and
once onset of CRS is detected.

96. The method of clause 79, wherein the first pharmaceutical composition is administered to the subject:
after the CAR T-cells are administered to the subject; and
once onset of CRS is detected by an increased level of one or more of cytokine IL-1α, IL-β, IL-2, IL-4, IL-6, IL-7, IL-8, IL-10, IL-12, IFN-γ, TNF-α, IP-10, MCP-1, MIP-1, RANTES, and GM-CSF.

97. The method of clause 79, wherein the first pharmaceutical composition is administered to the subject:
after the CAR T-cells are administered to the subject; and
once onset of CRS is detected by an increased level of one or more of inflammatory markers associated with CRS CRP (C-reactive protein) and ferritin.

98. The method of clause 79, wherein the first pharmaceutical composition is administered for a period of about 1 day to about 30 days.

99. The method of clause 79, wherein the first pharmaceutical composition is administered for a period of about 7 days to about 14 days.

100. The method of clause 79, wherein the first pharmaceutical composition is administered starting one day before administration of the CAR T-cells and continued for a period of at least about 14 days.

101. The method of clause 79, further comprising administering the first pharmaceutical composition to the subject before administration of the CAR T-cells.

102. The method of clause 79, wherein the severity measurements are event grades, event duration, event incidence, incidence of ICU or hospital stays, duration of ICU or hospital stays, onset timing, mortality, interference with antibiotics or other supportive medications, or combinations thereof.

103. The method of clause 79, wherein the CAR T-cells are autologous.

104. The method of clause 79, wherein the CAR T-cells are allogenic.

105. The method of clause 79, wherein the first pharmaceutical composition further comprises at least one excipient, at least one filler, at least one disintegrant, at least one binder, at least one wetting agent, at least one lubricant, at least one glidant, at least one preservative agent, at least one flavoring agent, at least one antioxidant, or combinations thereof.

106. The method of clause 79, wherein the first pharmaceutical composition is formulated as a tablet, capsule, granule, powder, liquid, suspension, gel, syrup, slurry, suppository, patch, nasal spray, aerosol, injectable, implantable sustained-release formulation, or mucoadherent film.

107. The method of clause 79, wherein the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

108. The method of clause 79, wherein the administering comprises oral delivery or intravenous injection (IV) delivery.

109. The method of clause 79, wherein the beraprost or pharmaceutically acceptable salt thereof is present in a unit dose of the first pharmaceutical composition in an amount of about 1 microgram to about 100 micrograms.

110. The method of clause 79, wherein the administering comprises delivering the first pharmaceutical composition to the subject at an amount of beraprost or a pharmaceutically acceptable salt thereof at least about 0.1 microgram.

111. The method of clause 79, wherein the administering comprises delivering the first pharmaceutical composition to the subject at an amount of beraprost or a pharmaceutically acceptable salt thereof at about 0.1 microgram to about 5000 micrograms.

112. The method of clause 79, wherein the administering comprises delivering the first pharmaceutical composition to the subject at an amount of beraprost or a pharmaceutically acceptable salt thereof at about 15 micrograms to about 90 micrograms.

113. The method of clause 79, wherein the administering comprises delivering the first pharmaceutical composition to the subject at an amount of beraprost or a pharmaceutically acceptable salt thereof at about 60 micrograms to about 360 micrograms.

114. The method of clause 79, wherein the CAR T-cells are Abecma (idecabtagene vicleucel), Breyanzi (isocabtagene maraleucel), Kymriah (tisagenlecleucel), Tecartus (brexucabtagene autoleucel), Yescarta (axicabtagene ciloleucel), Carvykti (ciltacabtagene autoleucel), Blincyto (blinatumomab), or combinations thereof.

115. A method of treating cytokine release syndrome, ICANS, or both, associated with CAR T-cell administration in a subject, the method comprising administering CAR T-cells and a first pharmaceutical composition to the subject, wherein:
the first pharmaceutical composition comprises at least an effective amount of beraprost or a pharmaceutically acceptable salt thereof, and
the first pharmaceutical composition is administered once onset of CRS is detected by an increased level of one or more of cytokine MIF, IL-5, IL-17A, IL-23, IFN-gamma, CXCL9/MIG, GCSF, VEGF-A, and TGF-beta, or one or more of inflammatory biomarker C-reactive protein (CRP) and ferritin.

116. The method of clause 115, wherein the pharmaceutically acceptable salt is beraprost sodium salt.

117. The method of clause 115, wherein the beraprost comprises at least one of BPS-314d, BPS-3141, BPS-315d, and BPS-315l.

118. The method of clause 115, wherein the beraprost is BPS-314d (esuberaprost sodium salt).

119. The method of clause 115, wherein:
the CAR T-cell administration is performed to treat cancer; and
the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma.

120. The method of clause 115, wherein:
the CAR T-cell administration is performed to treat cancer; and
the cancer is brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

121. The method of clause 115, wherein:
the CAR T-cell administration is performed to treat cancer; and
the cancer is a B-cell lymphoma.

122. The method of 115, wherein the subject is a mammal.

123. The method of clause 115, wherein the subject is a non-human primate, monkey, cat, dog, pig, cow, goat, horse, sheep, or rabbit.

124. The method of clause 115, wherein the subject is a human.

125. The method of clause 115, wherein the CAR T-cells and the first pharmaceutical composition are administered to the subject concurrently.

126. The method of clause 115, wherein the first pharmaceutical composition is administered to the subject after the CAR T-cells are administered to the subject.

127. The method of clause 115, wherein the first pharmaceutical composition is administered to the subject starting about 1 day, about 2 days, about 3 days, about 4 days, about 5 days, about 6 days, or about 7 days after the CAR T-cells are administered to the subject.

128. The method of clause 115, wherein the first pharmaceutical composition is administered to the subject starting about 3 days to about 7 days after the CAR T-cells are administered to the subject.

129. The method of clause 115, wherein the first pharmaceutical composition is administered for a period of about 1 day to about 30 days.

130. The method of clause 115, wherein the first pharmaceutical composition is administered for a period of about 7 days to about 14 days.

131. The method of clause 115, wherein the first pharmaceutical composition is administered starting one day before administration of the CAR T-cells and continued for a period of at least about 14 days.

132. The method of clause 115, further comprising administering the first pharmaceutical composition to the subject before administration of the CAR T-cells.

133. The method of clause 115, wherein the CAR T-cells are autologous.

134. The method of clause 115, wherein the CAR T-cells are allogenic.

135. The method of clause 115, wherein the first pharmaceutical composition further comprises at least one excipient, at least one filler, at least one disintegrant, at least one binder, at least one wetting agent, at least one lubricant, at least one glidant, at least one preservative agent, at least one flavoring agent, at least one antioxidant, or combinations thereof.

136. The method of clause 115, wherein the first pharmaceutical composition is formulated as a tablet, capsule, granule, powder, liquid, suspension, gel, syrup, slurry, suppository, patch, nasal spray, aerosol, injectable, implantable sustained-release formulation, or mucoadherent film.

137. The method of clause 115, wherein the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

138. The method of clause 115, wherein the administering comprises oral delivery or intravenous injection (IV) delivery.

139. The method of clause 115, wherein the beraprost or pharmaceutically acceptable salt thereof is present in a unit dose of the first pharmaceutical composition in an amount of about 1 microgram to about 100 micrograms.

140. The method of clause 115, wherein the administering comprises delivering the first pharmaceutical composition to the subject at an amount of beraprost or a pharmaceutically acceptable salt thereof at least about 0.1 microgram.

141. The method of clause 115, wherein the administering comprises delivering the first pharmaceutical composition to the subject at an amount of beraprost or a pharmaceutically acceptable salt thereof at about 0.1 microgram to about 5000 micrograms.

142. The method of clause 115, wherein the administering comprises delivering the first pharmaceutical composition to the subject at an amount of beraprost or a pharmaceutically acceptable salt thereof at about 15 micrograms to about 90 micrograms.

143. The method of clause 115, wherein the administering comprises delivering the first pharmaceutical composition to the subject at an amount of beraprost or a pharmaceutically acceptable salt thereof at about 60 micrograms to about 360 micrograms.

144. The method of clause 115, wherein the CAR T-cells are Abecma (idecabtagene vicleucel), Breyanzi (isocabtagene maraleucel), Kymriah (tisagenlecleucel), Tecartus (brexucabtagene autoleucel), Yescarta (axicabtagene ciloleucel), Carvykti (ciltacabtagene autoleucel), Blincyto (blinatumomab), or combinations thereof.

145. A method of treating cytokine release syndrome, ICANS, or both, associated with CAR T-cell administration in a subject, the method comprising administering CAR T-cells and a first pharmaceutical composition to the subject, wherein:
the first pharmaceutical composition comprises at least an effective amount of beraprost or a pharmaceutically acceptable salt thereof,
the subject experiences CRS, ICANS, Parkinsonism effects, or a combination thereof, and
the subject requires reduced treatment with at least one corticosteroid, tocilizumab, IL-6 receptor blocker, or combinations thereof relative to a similar subject receiving administered CAR T-cells but not receiving the administered first pharmaceutical composition.

146. The method of clause 145, wherein the pharmaceutically acceptable salt is beraprost sodium salt.

147. The method of clause 145, wherein the beraprost comprises at least one of BPS-314d, BPS-3141, BPS-315d, and BPS-315l.

148. The method of clause 145, wherein the beraprost is BPS-314d (esuberaprost sodium salt).

149. The method of clause 145, wherein:
the CAR T-cell administration is performed to treat cancer; and
the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma.

150. The method of clause 145, wherein:
the CAR T-cell administration is performed to treat cancer; and
the cancer is brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

151. The method of clause 145, wherein:
the CAR T-cell administration is performed to treat cancer; and
the cancer is a B-cell lymphoma.

152. The method of clause 145, wherein the subject is a mammal.

153. The method of clause 145, wherein the subject is a non-human primate, monkey, cat, dog, pig, cow, goat, horse, sheep, or rabbit.

154. The method of clause 145, wherein the subject is a human.

155. The method of clause 145, further comprising administering the first pharmaceutical composition to the subject before administration of the CAR T-cells.

156. The method of clause 145, wherein:
the first pharmaceutical composition is administered to the subject before the CAR T-cells are administered; and
the first pharmaceutical composition is administered to the subject after the CAR T-cells are administered to the subject.

157. The method of clause 145, wherein the CAR T-cells and the first pharmaceutical composition are administered to the subject concurrently.

158. The method of clause 145, wherein the first pharmaceutical composition is administered to the subject after the CAR T-cells are administered to the subject.

159. The method of clause 145, wherein the first pharmaceutical composition is administered to the subject starting about 1 day, about 2 days, about 3 days, about 4 days, about 5 days, about 6 days, or about 7 days after the CAR T-cells are administered to the subject.

160. The method of clause 145, wherein the first pharmaceutical composition is administered to the subject starting about 3 days to about 7 days after the CAR T-cells are administered to the subject.

161. The method of clause 145, wherein the first pharmaceutical composition is administered to the subject:
after the CAR T-cells are administered to the subject; and
once onset of CRS is detected.

162. The method of clause 145, wherein the first pharmaceutical composition is administered to the subject:
after the CAR T-cells are administered to the subject; and
once onset of CRS is detected by an increased level of one or more of cytokine IL-1α, IL-β, IL-2, IL-4, IL-6, IL-7, IL-8, IL-10, IL-12, IFN-γ, TNF-α, IP-10, MCP-1, MIP-1, RANTES, and GM-CSF.

163. The method of clause 145, wherein the first pharmaceutical composition is administered to the subject:
after the CAR T-cells are administered to the subject; and
once onset of CRS is detected by an increased level of one or more of cytokine IL-6, IL-10, IFN-γ, and TNF-α.

164. The method of clause 145, wherein the first pharmaceutical composition is administered for a period of about 1 day to about 30 days.

165. The method of clause 145, wherein the first pharmaceutical composition is administered for a period of about 7 days to about 14 days.

166. The method of clause 145, wherein the first pharmaceutical composition is administered starting one day before administration of the CAR T-cells and continued for a period of at least about 14 days.

167. The method of clause 145, wherein the CAR T-cells are autologous.

168. The method of clause 145, wherein the CAR T-cells are allogenic.

169. The method of clause 145, wherein the first pharmaceutical composition further comprises at least one excipient, at least one filler, at least one disintegrant, at least one binder, at least one wetting agent, at least one lubricant, at least one glidant, at least one preservative agent, at least one flavoring agent, at least one antioxidant, or combinations thereof.

170. The method of clause 145, wherein the first pharmaceutical composition is formulated as a tablet, capsule, granule, powder, liquid, suspension, gel, syrup, slurry, suppository, patch, nasal spray, aerosol, injectable, implantable sustained-release formulation, or mucoadherent film.

171. The method of clause 145, wherein the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

172. The method of clause 145, wherein the administering comprises oral delivery or intravenous injection (IV) delivery.

173. The method of clause 145, wherein the beraprost or pharmaceutically acceptable salt thereof is present in a unit dose of the first pharmaceutical composition in an amount of about 1 microgram to about 100 micrograms.

174. The method of clause 145, wherein the administering comprises delivering the first pharmaceutical composition to the subject at an amount of beraprost or a pharmaceutically acceptable salt thereof at least about 0.1 microgram.

175. The method of clause 145, wherein the administering comprises delivering the first pharmaceutical composition to the subject at an amount of beraprost or a pharmaceutically acceptable salt thereof at about 0.1 microgram to about 5000 micrograms.

176. The method of clause 145, wherein the administering comprises delivering the first pharmaceutical composition to the subject at an amount of beraprost or a pharmaceutically acceptable salt thereof at about 15 micrograms to about 90 micrograms.

177. The method of clause 145, wherein the administering comprises delivering the first pharmaceutical composition to the subject at an amount of beraprost or a pharmaceutically acceptable salt thereof at about 60 micrograms to about 360 micrograms.

178. The method of clause 145, wherein the subject does not require treatment with at least one corticosteroid.

179. The method of clause 145, wherein the subject does not require treatment with tocilizumab.

180. The method of clause 145, wherein the subject does not require treatment with IL-6 receptor blocker.

181. The method of clause 145, wherein the CAR T-cells are Abecma (idecabtagene vicleucel), Breyanzi (isocabtagene maraleucel), Kymriah (tisagenlecleucel), Tecartus (brexucabtagene autoleucel), Yescarta (axicabtagene ciloleucel), Carvykti (ciltacabtagene autoleucel), Blincyto (blinatumomab), or combinations thereof.

182. A kit comprising:
a first container containing a first pharmaceutical composition comprising at least an effective amount of beraprost or a pharmaceutically acceptable salt thereof;
a second container containing a second pharmaceutical composition comprising at least one corticosteroid, tocilizumab, IL-6 receptor blocker, or combinations thereof; and instructions for the administration of the first pharmaceutical composition, the second pharmaceutical composition, and CAR T-cells to a subject.

183. The kit of clause 182, further comprising a third container containing CAR T-cells.

184. The kit of clause 182, further comprising a fourth container containing water or an aqueous solution.

185. The method or kit of any of the preceding clauses, wherein the beraprost is BPS-314d.

Further aspects of the invention are also described in the following numbered clauses:

1A. A method of treating cytokine release syndrome (CRS), immune effector cell-associated neurotoxicity syndrome (ICANS), or both associated with CAR T-cell administration in a subject, the method comprising administering to the subject a population of CAR T-cells, and a first pharmaceutical composition; wherein:
the first pharmaceutical composition comprises at least an effective amount of beraprost, a beraprost isomer, or a pharmaceutically acceptable salt thereof, and
the first pharmaceutical composition does not reduce a cell killing mediated by the population of CAR T-cells by more than about 5%.

2A. A method of treating cytokine release syndrome, ICANS, or both, associated with CAR T-cell administration in a subject, the method comprising administering CAR T-cells, a first pharmaceutical composition, and a second pharmaceutical composition to the subject, wherein:
the first pharmaceutical composition comprises at least an effective amount of beraprost or a pharmaceutically acceptable salt thereof, and
the second pharmaceutical composition comprises at least one corticosteroid, tocilizumab, IL-6 receptor blocker, or combinations thereof.

3A. The method of clause 2A wherein the corticosteroid is dexamethasone.

4A. The method of clauses 2A-3A, wherein the subject requires reduced treatment with the second pharmaceutical composition relative to a subject who does not receive the first pharmaceutical composition.

5A. A method of treating cytokine release syndrome, ICANS, or both, associated with CAR T-cell administration in a subject can include administering CAR T-cells and a first pharmaceutical composition to the subject, wherein: the first pharmaceutical composition comprises at least an effective amount of beraprost, a beraprost isomer, or a pharmaceutically acceptable salt thereof, and the first pharmaceutical composition is administered once onset of CRS is detected by an increased level of one or more of cytokine MIF, IL-5, IL-17A, IL-23, CXCL9/MIG, GCSF, VEGF-A, and TGF-β, CCL2, CXCL9, CXCL-10, VEGF, CCL3, GCSF, CRP (C-reactive protein) and ferritin.

6A. A method of treating cytokine release syndrome and ICANS, or ICANS, associated with CAR T-cell administration in a subject can include administering CAR T-cells and a first pharmaceutical composition to the subject, wherein: the first pharmaceutical composition comprises at least an effective amount of beraprost, a beraprost isomer, or a pharmaceutically acceptable salt thereof, and the first pharmaceutical composition is administered once onset of CRS is detected by an increased level of one or more of cytokine MIF, IL-5, IL-17A, IL-23, IFN-γ, CXCL9/MIG, GCSF, VEGF-A, and TGF-β, CCL2, IL-2, IL-6, IL-8, IL-10, IFN-γ, TNF-α, CXCL9, CXCL-10, VEGF, CCL3, GCSF, and GMCSF, CRP (C-reactive protein) and ferritin.

7A. A method of treating cytokine release syndrome, ICANS, or both, associated with CAR T-cell administration in a subject, the method comprising administering CAR T-cells and a first pharmaceutical composition to the subject, wherein:
the first pharmaceutical composition comprises at least an effective amount of beraprost or a pharmaceutically acceptable salt thereof, and
the subject experiences reduced ICANS effects, Parkinsonism effects, or both, relative to a similar subject receiving administered CAR T-cells but not receiving the administered first pharmaceutical composition.

8A. The method of any of clauses 1A-7A wherein the Beraprost is BPS-314d (esuberaprost sodium salt).

9A. A method of treating cytokine release syndrome, ICANS, or both, associated with CAR T-cell administration in a subject can include administering CAR T-cells and a first pharmaceutical composition to the subject, wherein: the first pharmaceutical composition comprises at least an effective amount of BPS-314d (esuberaprost sodium salt), or a pharmaceutically acceptable salt thereof, and the first pharmaceutical composition is administered once onset of CRS is detected by an increased level of one or more of cytokine Il-6, IFN-Y and TNF-alpha.

10A. The method of any of clauses 1A-9A, wherein the CAR T-cell administration is performed to treat cancer; and
the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, or multiple myeloma, brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

11A. The method of any of clauses 1A-10A, wherein the CAR T-cells are autologous CAR T-cells or are allogeneic CAR T-cells.

### EXAMPLES

### Example 1: Evaluation of mouse model of CRS

This study will contain 3 groups—placebo control, positive control (tocilizumab or dexamethasone), and CTO1681 treated. The CTO1681 treated groups would have multiple arms for a dose-response determination in a murine model of CRS. These dosages will provide the information required for the secondary in vivo models of CAR-T therapy associated CRS treatment.

Humanized mice (expressing human PBMCs—HU-PBMC NSG™; commercially available from The Jackson Laboratory; Bar Harbor, ME, USA) will be given either control treatments or 1 of 5 doses of CTO1681 prior to CRS induction. To induce CRS, treated mice will be administered the antibody OKT3 intraperitoneally (IP), (anti-CD3 monoclonal antibody) to induce CRS. Mice will be sacrificed 24-48 hours post CRS-induction and cytokine production will be quantified (peripheral and in tissues).

Results will show that mice receiving CTO1681 prior to CRS induction had lower undesired cytokine production than mice in the control group. Results will also show a reduction in the rapid, acute symptoms that occur in the mouse model within the first 48 hours. Longer models would show survival benefits, but this particular Example will not be conducted to that point.

### Example 2: Mouse Model of CAR T-Cell Therapy Induced CRS

This study will contain 3 groups-placebo control, dexamethasone control, and CTO1681 dose determined from the primary CRS mouse studies of Example 1. SCID or humanized mice (expressing human PBMCs—HU-PBMC NSG™) will be injected (IP) with Raji-luc tumor cells and observed for approximately three weeks for tumor growth. Tumor burden will be assessed via bioluminescence.

CAR-T cell treatment (IP) will be performed to solicit CRS (occurs approximately 2-3 days after CAR-T infusion). Controls and CTO1681 treatments (IP, BID for 7 days) will start approximately 5 hours prior to CAR-T cell transfer. At the end of the 7-day treatment regimen tumor burdens will be assessed via bioluminescence and mice sacrificed for gross histopathology as well as peripheral and tissue cytokine concentrations determined.

Results will show that mice receiving either dexamethasone or CTO1681 had lower undesired cytokine production than mice in the control group, and that CTO1681 was superior to dexamethasone. While both the dexamethasone control and CTO1681 will demonstrate a reduction in cytokine levels, it is expected that there will be greater survival benefits to CTO1681 over dexamethasone.

### Example 3: Human Treatment with No Delay Period

A group of 50-100 human subjects having B-cell lymphoma will be divided into two groups—a control group and a CTO1681 treatment group. Both groups will receive infusion of CAR T-cells. The CTO1681 treatment group will receive CTO1681 starting a day prior to or with co-administration of the CAR T-cells and continuing daily for 7-15 days. Clinical signs of CRS and cytokines will be monitored daily for both groups. Results will show that subjects receiving CTO1681 had reduced CRS symptoms and lower undesired cytokine production than subjects in the control group.

### Example 4: Treatment with Predetermined Delay Period

A group of 80 human subjects having mantle cell lymphoma will be divided into two groups—a control group and a CTO1681 treatment group. Both groups will receive infusion of CAR T-cells. The CTO1681 treatment group will receive CTO1681 starting three days after administration with the CAR T-cells and continuing daily for eleven days. Cytokines will be monitored daily for both groups. Results will show that subjects receiving CTO1681 had reduced CRS symptoms and lower undesired cytokine production than subjects in the control group.

### Example 5: Treatment with Monitored Delay Period

A group of 40 human subjects having acute myeloid leukemia will be divided into two groups—a control group and a CTO1681 treatment group. Both groups will receive infusion of CAR T-cells. Cytokines will be monitored daily for both groups. The CTO1681 treatment group will start to receive CTO1681 upon detection of an increase in any one of cytokines IL-6, IFN-γ, and IL10. Treatment with CTO1681 will continue daily for ten days. Results will show that subjects receiving CTO1681 had reduced CRS symptoms and lower undesired cytokine production than subjects in the control group.

### Example 6: Kit for Treatment of Cancer

A box will be configured with a first container containing an effective amount of beraprost, a beraprost isomer, or a pharmaceutically acceptable salt thereof, and written instructions for the administration of the first pharmaceutical composition and CAR T-cells to a subject. The instructions can be printed on paper and placed within the box or can be a hyperlinked website having the written instructions. The box is combined with a second container containing autologous or allogeneic CAR T-cells. The box can optionally contain a third container containing water or an aqueous solution to dissolve the beraprost, a beraprost isomer, or a pharmaceutically acceptable salt thereof.

### Example 7: Ex Vivo Assessment of Cytokine Release Following CTO1681 Treatment

Cytokine release assays in normal human PBMC for analysis of CTO1681 down regulation of cytokine production were developed and performed per the parameters evaluated in earlier pilot work. For the current assays PBMC from 5 healthy donors were procured.

The assay began with pretreatment of rested cells with either a positive control drug (dexamethasone) or CTO1681. Following pre-treatment, cells were stimulated with either LPS or Poly (I:C). Twenty-four to forty-eight hours post-stimulation supernatants were collected and tested both for viability (48 hours) as well as concentrations of 29 different cytokines (24 hours.). Viability assessments confirmed the lack of CTO1681-associated cell cytotoxicity. Statistical comparisons in individual donor results were conducted between all test groups within each donor group (between individual stimulant-treatment pairs) to determine efficacy of stimulation as well as any potential CTO1681 effects on cytokine production. Additionally, statistical analysis was performed on pooled donor data for each cytokine stimulant-treatment pair to comprehensively determine cytokine suppression effects of CTO1681 for each cytokine.

Both LPS and Poly (I:C) stimulated PBMC from all five donors but there were some noted variabilities. Both stimulants failed to induce significant stimulation (stimulated cells vs. unstimulated control cells) in various cytokines from various donors. Likewise, there were donor-specific variabilities in the level of stimulation notably more often in Donor 5 who had a lower level of stimulation across all cytokines.

While these noted variations in cytokine responses were an unpredictable, unintentional result, this phenomenon does adequately represent the naturally observed variation in human immune responses. Taken together both as individual donor and pooled responses, CTO1681 treatment demonstrated significant reduction in 21 different cytokines detailed in the following Table 1, where the ** symbol indicates the 21 cytokines.

TABLE 1
Cytokine Responses

| | |
|---|---|
| LPS | Poly(I:C) |

| Cytokine | DEX | CTO1681 | DEX | CTO1681 |
|---|---|---|---|---|
| ** IL-1α | YES | YES | YES | YES |
| ** IL-1β | YES | YES | YES | YES |
| IL-2 | YES | NO | YES | NO |
| ** IL-4 | YES | YES | NO | YES |
| ** IL-5 | YES | YES | YES | YES |
| IL-6 | YES | NO | YES | NO |
| IL-7 | N/A | N/A | N/A | N/A |
| IL-8 | NO | NO | NO | NO |
| ** IL-9 | YES | YES | YES | NO |
| IL-10 | YES | NO | YES | NO |
| ** IL-12 | YES | YES | YES | YES |
| ** IL-13 | YES | YES | YES | YES |
| ** IL-15 | YES | YES | YES | YES |
| ** IL-17 | YES | YES | YES | NO |
| ** IL-18 | YES | YES | YES | YES |
| ** TNF α | YES | YES | YES | YES |
| ** CCL2 | YES | YES | NO | YES |
| (MCP-1) | | | | |
| ** CCL3 | YES | YES | NO | NO |
| (MIP1 α) | | | | |
| ** CCL5 | YES | YES | YES | YES |
| (RANTES) | | | | |
| ** CXCL9 | YES | YES | YES | YES |
| (MIG) | | | | |
| ** CXCL10 | YES | YES | YES | YES |
| (IP-10) | | | | |
| ** IFN-γ | YES | YES | YES | YES |
| ** IFN-α2 | YES | YES | YES | YES |
| GCSF | NO | NO | NO | NO |
| ** GM-CSF | YES | YES | N/A | N/A |
| ** FGF | YES | YES | NO | NO |
| ** PDGF | YES | YES | YES | YES |
| VEGF | N/A | N/A | N/A | N/A |
| TGFβ | NO | NO | NO | NO |

These 21 include 10 cytokines not previously identified by Gemmus Pharma, Inc. studies. Further, this work indicated that IL-2, IL-6, IL-8, and IL-10 signals were not significantly reduced in this ex vivo assay. It is important to note, that these four cytokines were identified as reduced by CTO1681 treatment in Gemmus Pharma's in vivo influenza (H5N1) therapeutic studies. Moreover, IL-2, IL-6, and IL-10 were reduced by CTO1681 treatment in current CytoAgents in vivo influenza (H1N1) studies. The differences in the observed reductions in in vivo work versus ex vivo work highlights an important aspect of the inherent differences in the assays with the caveat that a negative result in ex vivo does not always equate a negative result in vivo. These results can be surprising and unexpected at times.

The final cytokine measured in these assays not analyzed in previous Gemmus Pharma, Inc. work was IFN-α. IFN-α is a type I interferon that is tightly linked to the antiviral response of the immune system which is not generally associated with suppression through suppression of NFkB induction. CTO1681 had a significant reduction of the very low levels of IFN-α produced in this ex vivo system. This does not directly relate to suppression of IFN-α antiviral activity (IFN-α was not suppressed during earlier in vivo CTO1681influeza treatment studies). Altogether, data from Gemmus Pharma, Inc studies and the present work here confirm that the production of approximately 25 different cytokines are down regulated through treatment with CTO1681.

### Example 8: Cellular Targets—Normal Human PBMC

Cytokine release assays were performed in normal human PBMC from five donors of mixed age, race, and gender. Normal human PBMC were obtained from Lonza's extensive catalog of cellular reagents. Cells were thawed according to manufacturer's instructions, washed in complete growth media, and assessed for viability using trypan blue staining. A stock cell solution of 2×10⁶ cells/mL was suspended in complete growth media for allocation into black-walled plate cell wells (2×10⁵/well) for the assay. Cells were rested at 37° C., 5% CO₂ for 1 hour.

Treatment, Stimulation, Cell Harvest and Cell Viability Measures

After resting the cells, 100 μl of CTO1681 was added to appropriate wells at a final dosing concentration of 750 M. Vehicle and dexamethasone (final dosing concentration of 1 M) were also added at 100 μl respectively to their appropriate wells, and all were incubated at 37° C. with 5% CO₂ for 15 minutes. For cell stimulation, 20 μL of the 1.0 ng/mL LPS solution or the 250 g/ml Poly (I:C) solution were added to appropriate wells according to the group designation. Twenty-four hours later 100 μL of supernatant were collected from all wells and stored for cytokine analysis. Approximately four hours before final harvest, Alamar Blue dye was added to all wells. At forty-eight hours post stimulation all samples were collected, and supernatants tested for cell viability. When added to cells, AlamarBlue is modified by the reducing environment of viable cells and becomes highly fluorescent. Hence, increased fluorescence after cell staining indicates increased viability. Alamar blue viability assessments demonstrated no cytotoxicity when cells were treated with CTO1681 in Donors 1-5. Likewise, no signs of cytotoxicity were observed by the stimulation of cells with LPS or Poly (I:C).

Cytokine Readouts, End Results, and Conclusions

Cytokines were assessed on 24-hour post-stimulation collected samples on a MAGPIX instrument using Luminex multiplex technology via multiplex kits from Millipore. Concentrations of each cytokine were assessed based on a standard curve. Sample values collected by the multiplex analysis below the limit of detection were not included in final determinations. Undiluted donor IL-6 concentrations were beyond the linearity of the assay and hence the valuations were too high for the typical standard curve. Therefore IL-6 samples were re-run at a 1:5 dilution. The determined cytokine concentrations (pg/ml) results of each cytokine were plotted as individual donor stimulant-treatment pairs. Statistical comparisons on all cytokines individual donor results were conducted between control groups via a one-way ANOVA with multiple comparisons. ANOVIA analyses were between the stimulated groups (LPS only, Poly(I:C) only) and the controls (media only, dex, and vehicle) to determine efficacy of stimulation and dexamethasone positive control, as well as any potential vehicle effect. An ordinary one-way t-test was then used to compare the vehicle group to the CTO1681 stimulation to determine if there was an effect with the test item. The stimulant-treatment pairs of all five donors as pooled were plotted, single means to evaluate the overall efficacy of CTO1681 cytokine suppression. Statistical analysis (Wilcoxon matched-pairs signed rank test) was performed on the pooled donor results for each cytokine plotted as well. Cytokine suppression end results were determined by evaluating both the pooled results as well as the individual donor results. CTO1681 reduced 21 of the 29 cytokines analyzed in this work.

Observations from Ex Vivo Treatment Results

Effective stimulation of the PBMCs via LPS or Poly(I:C) was determined by comparison of the media control group to the stimulation group (non-treatment). Both mitogens successfully stimulated the majority of the cytokines, however there were variabilities both between donors and between cytokines. VEGF and TL-7 failed to stimulate consistently with either LPS or Poly(I:C) to a level above the lower limits of the standard curves and hence were not computed in the pooled analysis. The failure to stimulate is likely an artifact of the ex vivo assay which must be run with relatively short windows of observation as well as the lack of the complexities of a complete in vivo system. Additional cytokines while successfully stimulated, didn't achieve stimulation to a level of significant value as compared to unstimulated media wells. Statistically significant increases in either LPS or Poly(I:C) were detected in a minority of donors in the TGF-β, TL-2 results. Statistical significance was not achieved in the Poly(I:C) groups for GM-CSF, TL-8, IL-17A, CXCL10/IP-10, or CCL5/RANTES. Variability between the donor's general levels of stimulation was also a notable observation. Overall, Donor 5 had a much lower level of stimulation across all cytokines when treated with both Poly(I:C) and LPS. Due to this lack of stimulation, Donor 5 data was not included in pooled and final analysis for IL-2, TL-4, IL-5, IL-9, IL-15, IL-12, IL-13, IL-17, IL-18, GM-CSF, and PDGF. Donor 5 stimulation for cytokines IL-10 and CXCL10/IP-10 was above the lowest limit of detection but the values were magnitudes (1-3 logs) below the other 4 donors hence while Donor 5 data was not included in the pooled analysis for these two cytokines, individual results were still considered in final determinations for CTO1681 efficacy. Similarly, Donor 1 TGF-β results were all below the lower limit threshold from the standard curve so data from this donor for this cytokine were not included in the overall pooled analysis of CTO1681 efficacy. Donor 4 did have a notable lower level of overall stimulation in several cytokines (but not below the threshold for inclusion in calculations). Taken together these differences highlight the natural variation seen in generalized immune responses to mitogens within a population likely due to genetic differences between the subjects.

Much like the observations noted for the variabilities seen between groups and donors for successful stimulation, a similar unpredictability was observed with treatment group results within both stimulation methods and donors as well. CTO1681 was capable of decreasing concentration in a few cytokines on which dexamethasone had no effect. Overall, CTO1681 suppressed cytokine production in 21 of the 29 cytokines evaluated. Only three cytokines, GCSF, TGF-β and IL-8, experienced a lack of suppressed production with both CTO1681 and dexamethasone treatments, despite successful stimulation in both LPS and Poly(I:C) induced cells. Likewise, in some instances dexamethasone failed to suppress cytokine production while CTO1681 successfully reduced produced cytokine concentrations (Poly(I:C)-stimulated cells-IL-4, CCL2/MCP1), as well as the vice versa with CTO1681 failing to suppress cytokine production while dexamethasone successfully reduced the cytokine concentrations (Poly(I:C)-stimulated cells—IL-17, IL-9). Furthermore, in some instances, both dexamethasone and CTO1681 demonstrated significant reductions in cytokine production in one stimulation mitogen (LPS) but not the other (Poly(I:C)), as observed in FGF and CCL3/MIP1a. Undeniably, the complexities of assessing the study in its entirety, controls versus CTO1681 in the two different stimulants, in five donors, and 29 different cytokines, are quite formidable. Ultimately, conclusions based on evaluations of the pooled and individual donor data indicate CTO1681 suppression of cytokine production in both stimulation methods in the following cytokines—IL-1-α, IL-1β, IL-4, IL-5, IL-12,p70, IL-13, IL-15, IL-18, TNF-α, CCL2/MCP-1, CCL5/RANTES, CXCL9/MIG, CXCL10/IP-10, IFN-γ, IFN-α, and PDGF; and observed suppression in cytokines from LPS stimulation alone—IL-9, IL-17, CCL3/MIP1a, GM-CSF, and FGF.

There are many operational factors to consider in the interpretation of the mechanisms and actions behind these tabulated results. Each stimulant triggers responses through different cellular receptors which in turn can then have different timing in the subsequent cellular cascade of events. Further, the window of analysis for this study—24 hours, while necessary for the nature of this ex vivo study, likely does not provide enough time for all the evaluated cytokines' production to be initiated or reach a level of significant expression. Additionally, in lacking a complete biological system, recruitment of immunological factors that can play a role in the different cytokine responses are also not present in the entirety that exists in an intact physiological system. Hence negative results should be observed with this knowledge and require the awareness that in vivo results could be somewhat different. For instance, in this ex vivo study, IL-2, IL-6, IL-8 and IL-10 were all negative for CTO1681 suppression. However, in concurrent data from in vivo lethal influenza H1N1 studies (and previous Gemmus Pharma Inc. H5N1 in vivo studies) CTO1681 significantly suppressed the production of these cytokines.

CTO1681 suppression of ex vivo IFN-α activity was surprising and unexpected. IFN-α is a type 1 interferon not typically associated with the same pathways or patterns of standard proinflammatory cytokines, or more pointedly, activity through NFκB induction. Type 1 interferons are an important part of the antiviral immune response. Further, type I interferons are not typically associated with NFκB production as IFN-α is produced from different promoters and transcriptional elements than standard proinflammatory cytokines. The pathway for IFN-α, from cellular receptors to feedback loops, is entirely different from proinflammatory cytokines. Inclusion of IFN-α in this study was initially incorporated as a safety, not an efficacy measure. The results seen here are the reverse of the ex vivo/in vivo phenomenon mentioned above for IL-2, IL-6, et. al. While IFN-α suppression by CTO1681 is apparent in the data in these ex vivo studies, our in vivo data indicates the contrary, or no significant repression of IFN-α concentrations by CTO1681 treatment during lethal H1N1 influenza infections. Of importance when evaluating these two contrasting results is the recognition of the substantial difference in the amount of INF-α produced in ex vivo stimulated PBMC versus active viral replication induced INF-α levels in vivo. LPS stimulated INF-α production in PBMC was <101 pg/ml, while Poly(I:C) stimulated levels were approximately 101 pg/ml. H1N1 lethally infected mice had approximately 103 pg/ml INF-α detected in BALF fluids. The nominal reduction in the ex vivo production of INF-α (albeit statistically significant) was less than 2-fold. At the levels of INF-α produced during active viral replication, this small reduction would not be a significant change in BALF INF-α. INF-α production occurs in two phases, early and late, with the late production correlated with the high levels of INF-α associated with viral infections. One of the transcription factors responsible for a pronounced portion of INF-α production in the early phase is IRF7. TRF7 has an NFkB response element in its promoter. Mechanistically, this is the likely means that CTO1681 indirectly influences INF-α production, having significant yet minimal in magnitude effects on the type 1 interferon produced as observed in this ex vivo assay.

In summary, this data and information demonstrate a broad efficacy of CTO1681 cytokine suppression of 25 different cytokines identifying new cytokines (10 as compared to previous Gemmus Pharma, Inc. data) affected through ex vivo treatment of stimulated PBMC with the molecule.

### Example 9: Kits Containing Beraprost and Tocilizumab

A box will be configured with a first container containing a first pharmaceutical composition containing an effective amount of beraprost, a beraprost isomer, or a pharmaceutically acceptable salt thereof, a second container containing a second pharmaceutical composition containing an effective amount of Tocilizumab, and written instructions for the administration of the first pharmaceutical composition, second pharmaceutical composition, and CAR T-cells to a subject. The instructions can be printed on paper and placed within the box or can be a hyperlinked website having the written instructions. The box is combined with a third container containing autologous or allogeneic CAR T-cells. The box can optionally contain a fourth container containing water or an aqueous solution to dissolve the beraprost, a beraprost isomer, or a pharmaceutically acceptable salt thereof and the Tocilizumab.

### Example 10: Use of Beraprost and Tocilizumab in Combination

Subjects suffering from cancer will be treated with orally-administered beraprost, a beraprost isomer, or a pharmaceutically acceptable salt thereof one day before being administered CAR T-cells. The beraprost, a beraprost isomer, or a pharmaceutically acceptable salt thereof will be continued for an additional at least 14 days. Tocilizumab and/or corticosteroid can be added as directed by the physician but will be needed at a lower amount relative to similar subjects not receiving beraprost, a beraprost isomer, or a pharmaceutically acceptable salt thereof. Improved clinical outcomes will be observed from the combination as compared to other subjects. The subjects will be observed to have reduced ICANS effects, and a lower average incidence of hospital stays and lower average duration of hospital stays.

### Example 11: Phase 1b Study of Preventing or Reducing CAR T-Cell-Induced Toxicities

The Phase 1b of the study is a multicenter, open-label, dose-escalating, safety and pharmacokinetic (PK) study of multiple ascending doses (MAD) of CTO1681 in patients with B-NHL who receive commercially available CD19 directed CAR T-cell therapy. This portion of the study will be conducted using a Bayesian optimal interval (BOIN) design to inform dose escalation among cohorts of patients with B-NHL. It will include the following sequential steps for every patient in each cohort: screening, enrollment, pretreatment with lymphodepleting (LD) chemotherapy, initiation of study drug (CTO1681), CAR T cell infusion, continuing treatment with CTO1681 (for a total of 15 days), and safety follow-up. Patients with CRS or ICANS that has not resolved by Day 15 may continue to receive treatment for up to an additional 13 days (total of 28 days) at the discretion of the Investigator with the approval of the Medical Monitor and Sponsor. Timing of treatment with CTO1681 will occur according to the following schedule in relation to LD and CAR T cell infusion:

Days −12 to −3: LD chemotherapy (if warranted per CAR T-cell manufacturer's package insert [PI])

Day −1: Initiate study drug (CTO1681) approximately 24 hours before CAR T-cell infusion. Study drug will be continued orally (PO), 3 times daily (TID) for 15 days.

The total daily dose of CTO1681 will be 30, 60, or 90 μg, depending on the cohort into which each patient is enrolled. Study drug must be taken within 30 minutes after a meal or snack.

Day 1: CAR T-cell infusion (per CAR T-cell manufacturer's PI). Patients will be monitored to document tumor response for up to 6 months.

A BOIN algorithm will be implemented to advise on dose escalation. The algorithm will target identification of the maximum tolerable dose (MTD) with a probability of toxicity less than 33%. The dose-limiting toxicity (DLT) observation period is defined as 42 days following the first dose of study drug.

Three dose cohorts will be considered with a maximum of 27 patients. The first dose level (30 μg) will be enrolled with a cohort of 3 patients. Dose levels of 60 and 90 μg will be initially enrolled with 6 patients. Total enrollment per dose level cohort will be a maximum of 9 patients.

The MTD is defined as the maximum dose achieved before a >33% toxicity rate is observed. The recommended Phase 2 dose (RP2D) is the MTD or the highest dose where MTD stopping did not occur. The target toxicity rate is ≤33%. See Table 2.

TABLE 2
Dose escalation and stopping rules

| Dose | No. patients | Max number | Dose | MTD |
|---|---|---|---|---|
| level | initial cohort | of patients | escalation rules | stopping rules |
| 30 ug | 3 | 9 | 0/3 DLT | ≥2/3 DLT |
| | | | ≤1/6 DLT | ≥3/6 DLT |
| | | | ≤2/9 DLT | ≥4/9 DLT |
| 60 ug | 6 | 9 | ≤1/6 DLT | ≥3/6 DLT |
| | | | ≤2/9 DLT | ≥4/9 DLT |
| 90 ug | 6 | 9 | ≤1/6 DLT | ≥3/6 DLT |
| | | | ≤2/9 DLT | ≥4/9 DLT |

Abbreviations: DLT = dose limiting toxicity; MTD = maximum tolerated dose.

### Example 12: Phase 2a Study

Phase 2a: The Phase 2a of the study will be conducted as a double-blind, randomized, placebo-controlled study and will enroll approximately 104 patients. Patients will be treated at the RP2D of CTO1681, which was established during Phase 1b, or with placebo. Patients will be randomized in a 1:1 ratio between the CTO1681 and placebo arms before receiving LD chemotherapy. If more than 1 commercially available CAR T cell product is carried forward from the Phase 1b portion of the study, attempts will be made to balance the multiple CAR T-cell products wherever feasible. Each patient will follow the same study steps as noted above for the Phase 1b portion. The duration of treatment will be determined based on findings and recommendations from the Phase 1b study following an end of Phase 1b interim analysis (for example, average days on treatment, number of clinician-initiated extension requests, and recommendations of the Safety Review Committee [SRC]). This portion of the study will include a PK substudy at selected sites. Patients enrolled in this portion of the study will be monitored for tumor response for up to 12 months following CAR T-cell infusion.

For both phase 1b and phase 2a studies, safety, efficacy, and biomarker assessments will be conducted at protocol-specified intervals per the Schedule of Events.

Study patients will be hospitalized for observation and management of CAR T-cell-related toxicities based on institutional guidelines. All CRS and ICANS grading will be according to American Society of Transplantation and Cellular Therapy (ASTCT) criteria. Detailed management guidelines for CRS and ICANS will be provided in the full protocol and will be consistent with standard of care management.

Currently commercially available CD19-directed CAR T-cell therapies for B-NHL, namely axicabtagene ciloleucel [YESCARTA®], tisagenlecleucel [KYMRIAH®], and lisocabtagene maraleucel [Breyanzi®] will be included in the Phase 1b portion of the study. Based on the interim analysis of the Phase 1b results, 1 or more of these CAR T-cell therapies will be carried forward to the Phase 2a study

### Example 13: Management of CRS and ICANS

CTO1681 should be continued in patients who develop CRS, except as noted under the protocol section for Management of DLTs. Detailed management guidelines for CRS and ICANS will be provided to the investigators to standardize care across the participating study sites. These guidelines will be consistent with the standard of care outlined in the CAR T-cell product package inserts, with the exception of the use of prophylactic steroids or tocilizumab.

### Example 14: Key Dose Limiting Toxicities

The key DLT criteria are:

Hypotension not attributed to underlying CAR T-cell therapy that is defined as documented, prolonged (≥2 hours) systolic blood pressure <90 mm Hg unresponsive to basic medical therapy (that is, fluid challenge, vasopressors).

Bleeding: Any clinically significant bleeding event (that is, excluding accidental cuts, periodontal, menstrual, or hemorrhoidal bleeding, or easily controlled epistaxis) including any episode of hemoptysis.

Hepatic dysfunction: Any occurrence of transaminase elevation of >5×upper limit of normal (ULN; Common Terminology Criteria for Adverse Events [CTCAE] Grade 3).

QTc prolongation: Development of QT interval corrected using Fridericia's formula (QTcF) ≥501 msec or a 60 msec change from baseline persisting for ≥5 minutes confirmed by repeat 12-lead electrocardiogram (ECG)/telemetry.

Ventricular tachycardia requiring urgent medical intervention (CTCAE Grade 3).

### Example 15: Management of Dose Limiting Toxicities

DLT of hypotension or hepatic dysfunction: CTO1681 will be interrupted until the DLT resolves. Should the DLT recur, CTO1681 will be discontinued.

DLT of bleeding: CTO1681 will be discontinued.

DLT of QTc prolongation or ventricular tachycardia: CTO1681 will be interrupted if patient develops Grade 3 or 4 CRS.

### Example 16: Objectives

Phase 1b:

Determine the RP2D and, as appropriate, MTD of CTO1681 in patients with B-NHL receiving CAR T-cell therapy

Determine the preliminary safety profile of CTO1681 in patients with B-NHL receiving CAR T cell therapy

Phase 2a:

Determine preliminary CTO1681 effectiveness in preventing or reducing CRS in comparison to placebo

Determine preliminary CTO1681 effectiveness in preventing or reducing ICANS in comparison to placebo

Determine the expanded safety profile of CTO1681 in patients with B-NHL receiving CAR T-cell therapy

Investigate the potential impact of CTO1681 on antitumor activity of CAR T-cell therapy in comparison to historical data and placebo.

### Example 17: Endpoints

Phase 1b:

Primary:

Adverse events (AEs), including DLTs, and laboratory abnormalities as characterized by type, frequency, timing, severity (graded per CTCAE v5.0), seriousness, and relationship to CTO1681

Exploratory:

Rate, severity, onset, and duration of CRS, as measured by CRS grade area under the curve (AUC) between initiation of CAR T-cell therapy and 30 days after initiation of CAR T-cell therapy

Rate, severity, onset, and duration of ICANS, as measured by ICANS grade AUC between initiation of CAR T-cell therapy and 30 days after initiation of CAR T-cell therapy

Days on treatment with CTO1681

Incidence of Grade ≥3 CRS and Grade ≥3 ICANS

Antitumor activity including overall response rate (ORR), duration of response (DOR), and progression-free survival (PFS) according to Investigator review, and overall survival (OS)

Frequency and duration of hospitalizations and intensive care unit (ICU) admissions due to CRS or ICANS

Levels and persistence of CAR-T cells

Use of tocilizumab, steroids, and other anti-cytokine therapies for treatment of CRS or ICANS

PK concentration profile of CTO1681

Biomarkers relating to the mechanism of action (MOA) of CTO1681 and immune response

Immune reconstitution

Phase 2a:

Primary:

Rate, severity, onset, and duration of CRS, as measured by CRS grade AUC between CAR T cell treatment initiation and 30 days after CAR T-cell treatment initiation

Secondary:

Additional assessments of CRS incidence and severity:

Maximum grade CRS

Incidence of Grade ≥2 CRS

Incidence of Grade ≥3 CRS

Duration of Grade ≥1 CRS

Rate, severity, onset, and duration of ICANS, as measured by ICANS grade AUC between CAR T cell treatment initiation and 30 days after CAR T-cell treatment initiation

Additional assessments of ICANS incidence and severity:

Maximum grade ICANS

Incidence of Grade ≥2 ICANS

Incidence of Grade ≥3 ICANS

Duration of Grade ≥1 ICANS

Frequency and duration of hospitalizations and ICU admissions due to CRS or ICANS

Use of tocilizumab, steroids, and other anti-cytokine therapies for treatment of CRS or ICANS

Antitumor activity including ORR, DOR, and PFS according to Investigator review, and OS

Exploratory:

Levels and persistence of CAR-T cells

PK concentration profile of CTO1681

Biomarkers relating to MOA of CTO1681 and immune response

Immune reconstitution

### Example 18: Number of Patients (Planned)

Phase 1b: 15 up to 27

Phase 2a: 104

### Example 19: Inclusion Criteria

1. Age 18 years or older.

2. Undergone leukapheresis and is scheduled to receive protocol-specified commercially available CD19-directed CAR T cell therapy for B NHL without corticosteroid prophylaxis for CRS. Patients eligible for study must have relapsed or refractory DLBCL after at least two prior lines of systemic therapy.

3. Met all inclusion criteria for CAR T-cell therapy per institutional guidelines.

4. Adequate organ function defined as:
A. Serum creatinine ≤1.5×ULN or estimated glomerular filtration rate (eGFR) ≥60 mL/min/1.73 m2 per Cockroft-Gault formula.
B. Serum alanine aminotransferase (ALT)/aspartate aminotransferase (AST)≤2.5×ULN.
C. Bilirubin ≤2.0 m g/dL except Gilbert syndrome patients with total bilirubin ≥3.0×ULN and indirect bilirubin ≤1.5×ULN.
D. Left ventricular ejection fraction (LVEF) ≥40% on echocardiogram (ECHO) or multigated acquisition (MUGA) and no clinically significant pericardial effusion.
E. Platelets ≥50,000/mm3.
F. Absolute neutrophil count ≥1000/μL
G. Absolute lymphocyte count ≥100/μL

5. Documented measurable disease adequate to judge by Lugano criteria.

6. Eastern Cooperative Oncology Group performance status 0 to 1.

7. Female participants of childbearing potential and all male participants must agree to use Investigator-approved methods of birth control while on study drug and for 30 days thereafter.

8. Patients who are able to provide written informed consent before the predose procedures, or patients who have a legal representative capable of providing informed consent on their behalf.

### Example 20: Exclusion Criteria

1. Any cytotoxic chemotherapy within 14 days of leukapheresis.

2. Clinically significant malabsorption syndromes and swallowing difficulties (for example, odynophagia, dysphagia, gastroesophageal reflux disease) as per Investigator assessment.

3. Clinically significant electrolyte imbalance (for example, hypokalemia, hypomagnesemia) as per Investigator assessment.

4. Abnormal or clinically significant ECG abnormality at Screening and Baseline (Day 1), including but not limited to, a confirmed QTcF value >450 msec for males and >470 msec for females. Patients with QTcF readings that are borderline or difficult to interpret because of a condition such as bundle branch block, or in those where the T and U waves are superimposed will be excluded.

5. History of arrhythmia (for example, bradycardia, congenital long QT syndrome, atrial fibrillation) and/or requiring anticoagulation/antiplatelet treatment at therapeutic dose.

6. Any clinically significant (that is, active) cardiovascular disease, including cerebral vascular accident/stroke (<6 months before enrollment), myocardial infarction (<6 months before enrollment) or unstable angina, and congestive heart failure ≥New York Heart Association Classification Class III.

7. Uncontrolled thromboembolic events or recent severe hemorrhage.

8. Requirement for ongoing therapeutic doses of anticoagulant therapy, antiplatelet or fibrinolytic agents.

9. Patients who, in the opinion of the Investigator, would be unlikely to comply with study procedures or are otherwise unsuitable for enrollment.

### Example 21: Investigational Product, Dosage, and Mode of Administration

Phase 1b: CTO1681 will be administered as a 10-μg tablet. In Cohort 1, CTO1681 tablets will be administered PO TID for minimum of 15 days but no more than 28 days at a total daily dose of 30 μg. Cohorts 2 and 3 will receive CTO1681 PO TID for a minimum of 15 days but no more than 28 days at total daily doses of 60 and 90 μg, respectively. Neither intrapatient dose modification nor dose modification within a cohort is permitted.

Phase 2a: CTO1681 will be administered as 10-μg tablets, potentially multiple tablets per dose, depending on the dose determined by the Phase 1b results. The duration of treatment will also be determined by the Phase 1b results.

NOTE: As CTO1681 has a positive food effect, it should be administered within 30 minutes after completion of a meal or snack.

### Example 22: Duration of Study (Per Patient)

Phase 1b: approximately 7 months (including screening)

Phase 2a: approximately 13 months (including screening)

### Example 23: Reference Therapy, Dosage, and Mode of Administration

Phase 1b: none.

Phase 2a: Placebo tablets will be administered PO, TID in a manner identical to active treatment. Placebo tablets will be identical in size, shape, color, and packaging to CTO1681 10-μg tablets.

### Example 24: Safety Review

The SRC will be composed of study investigators, the Sponsor's medical representative (or qualified delegate), and ad hoc members as appropriate (for example, statistician, PK expert). The SRC will monitor the progress and safety of patients throughout the Phase 1b study. Regular systematic review of AEs will serve as the basis for pausing or prematurely stopping the study. Unexpected SAEs that are related to CTO1681 will be the primary criteria for pausing or stopping the study. If a CTO1681-related DLT occurs in >33% of patients within a cohort (excluding fever, hematological toxicities, and CRS or ICANS, or events not related to underlying disease or CAR T-cell therapy), this will indicate that the MTD has been reached and the study will be stopped.

During the Phase 1b portion of the study, the SRC will review the safety data after the completion of the DLT evaluation period (42 days after the first dose of CTO1681). In order to determine if dose escalation may occur for each dose level cohort according to the BOIN algorithm, the SRC will decide whether to proceed to the next cohort based on safety data the earlier of either 42 days from first dose of study drug in the last patient treated in the current cohort if the patient was treated for the maximum of 28 days, or 14 days after the last dose for the last patient treated in the current cohort if the last patient was treated for fewer than 28 days. The dose may be escalated, additional subjects may be enrolled (up to 9 per dose level), or the study may be stopped. Cumulative safety data will be reviewed at each SRC meeting. The cohort will be paused or stopped and the SRC will review and determine the relationship of the AE to CTO1681 if any of the pausing or stopping rules are met.

After either the completion of at least 1 month of follow-up since the first dose of CTO1681 administered in Phase 1b or early discontinuation from study for all patients enrolled in Phase 1b, an interim analysis will be performed to summarize the Phase 1b results. The SRC will advise on the advancement to the Phase 2a portion of the study and the selection of an RP2D on the basis of safety, including available antitumor response data. The SRC will also advise on the dose schedule and recommended CAR T-cell products for inclusion in Phase 2a.

The SRC may also advise on any necessary protocol amendments with respect to patient safety throughout the Phase 1b portion of the study.

An Independent Data Monitoring Committee (IDMC) composed of 2 CAR T/hematology oncology experts and 1 independent biostatistician will be established to monitor safety data during the Phase 2a portion of the study. The IDMC will perform planned safety data reviews after approximately 20, 40, 60, 80, and 104 patients have been followed at least 1 month from first exposure to study drug or have discontinued early from the study. The study will be paused or stopped if any of the prespecified pausing or stopping rules are met. An ad hoc meeting of the IDMC will be held in the event a death deemed by the Investigator and confirmed by the Sponsor is consistent with a known adverse effect of CTO1681, such as DLTs identified as pausing criteria during the Phase 1b portion of the study. In addition, the IDMC will meet more frequently, as necessary, to review serious AEs (SAEs) and provide recommendations on when to pause enrollment or terminate the study.

The IDMC will review the rates of complete response (CR) in accordance with a prespecified set of nonbinding antitumor response impact assessments, planned to occur during the 40, 60, and 80 patient safety data reviews. The assessments will include patients who have achieved CR, according to the Lugano Response Criteria have best antitumor response of progressive disease, have prematurely discontinued from the study, or have been followed for at least 3 months since first dose of CAR T-cell therapy. At each antitumor response impact assessment, for patients randomized to CTO1681, the CR rate and an exact Clopper-Pearson 95% CI will be computed to compare the upper bound of the CI to a benchmark rate that is derived from the lower boundary of the 6-month CR rate 95% CI from prior literature. The lower boundary will be selected at the completion of Phase 1b upon sponsor selection of CAR T cell therapies that will be included in the Phase 2a portion of the study. Study enrollment will not be paused for the planned safety data reviews or antitumor response impact analyses unless warranted according to the safety stopping rules.

A formal charter will be established to specify the rules and scope of responsibilities of the SRC and IDMC.

### Example 25: Criteria for Pausing or Stopping the Study

If any of the following events occur within 42 days after first treatment with study drug and are deemed by the Investigator as not related to underlying disease, CAR T-cell therapy, or an event or factor unrelated to study participation, the SRC (in Phase 1b) or the IDMC (in Phase 2a) will be immediately informed, and an ad hoc meeting scheduled to review all available safety data. The committee will then provide recommendations as to how to proceed.

Pausing Criteria

Phase 1b:

The CTO1681 DLT rate exceeds 33%:

Bleeding: Any clinically significant bleeding event (excluding accidental cuts, periodontal, menstrual, or hemorrhoid bleeding, or easily controlled epistaxis.

Hypotension: Prolonged and symptomatic (≥2 hours) systolic blood pressure <90 mm Hg unresponsive to basic medical therapy.

QTc prolongation: QTcF ≥501 msec or a 60 msec change from baseline persisting for ≥5 minutes.

Ventricular tachycardia (CTCAE Grade 3)

Transaminase elevation >5×ULN (CTCAE Grade 3)

Two or more patients in an assigned cohort experience a CTCAE Grade 4 toxicity (excluding events that may be attributed to disease progression/underlying disease or CAR T-cell therapy (such as fever, hematological toxicities, and CRS/ICANS).

Death that is study-related but not related to underlying disease or CAR T-cell therapy.

Phase 2a:

Death deemed by Investigator and confirmed by the Sponsor representative to be consistent with a known adverse effect of CTO1681, such as the DLTs identified as pausing criteria in Phase 1b.

After the safety review, if the cohort and/or study is deemed safe to proceed, recruitment and treatment will proceed as planned. If there are outstanding questions, the study may proceed in accordance with recommendations made by the SRC or the IDMC.

Stopping Criteria

Phase 1b:

The CTO1681 DLT rate exceeds 33%

CTCAE Grade 4 toxicity (unmanageable, unexpected) assessed by the Investigator and confirmed by the SRC and the Sponsor to be possibly, probably, or definitely related to CTO1681 (excluding CAR T-cell therapy-related toxicities such as fever, hematological toxicities, and CRS/ICANS).

Death resulting from a CTO1681 associated DLT not related to underlying disease, CAR T-cell therapy, or an extrinsic event (unrelated to the investigational product or study procedures). Specifically:

Fatal bleeding event.

Refractory hypotension resulting in death.

Refractory ventricular tachycardia resulting in death.

Phase 2a:

CTCAE Grade 4 toxicity (unmanageable, unexpected) occurring in >33% of dosed patients that is possibly, probably, or definitely related to CTO1681 (excluding fever, hematological toxicities, and CRS/ICANS).

Death resulting from a CTO1681-associated DLT not related to underlying disease, CAR T-cell therapy, or an extrinsic event (unrelated to the investigational product or study procedures). Specifically:

Fatal bleeding event.

Refractory hypotension resulting in death.

Refractory ventricular tachycardia resulting in death.

The Sponsor may decide to stop or make adaptations to the study based on recommendations of the SRC/IDMC and the totality of evidence. The SRC may recommend that the study be stopped if it is determined that patient safety may be compromised by continuing the study. The IDMC may recommend that the study be stopped based on safety or futility grounds.

### Example 26: Statistical Methods

Sample Size Considerations:

Phase 1b: The maximum sample size of 27 (maximum 9 per dose level) was selected to support the dose escalation and RP2D selection of CTO1681 dose level in accordance with the BOIN algorithm. Phase 1b safety results will contribute to determining the duration of treatment and CAR T-cell therapy to be continued in Phase 2a.

Phase 2a: This study was designed with sample size selected to provide sufficient power for the primary endpoint as well as a selection of secondary endpoints. While CRS grade AUC is identified as the primary endpoint, there is also interest in assessing the impact of CTO1681 on other measures of CRS severity to better understand the sensitivity of capturing the treatment effect.

Computing the per-patient CRS grade AUC as the sum of the daily CRS maximum (nonzero) grade CRS across the duration of the efficacy observation period (30 days following initiation of CAR T cell therapy), a sample size of 104 patients (52 each in the CTO1681 and placebo groups) provides over 85% power with a 1 sided type I error rate of 0.05 using a difference in means t-test to detect a true between group difference in mean AUC of 4.5 CRS grade days with a standard deviation of 8.

A sample size of 104 patients (52 per group) provides over 85% power with a 1-sided type I error rate of 0.05 using a Wilcoxon-Mann-Whitney test to establish a difference in ordinal variables under the assumption that with no treatment, 35% of CAR T-cell patients experience a maximum Grade 1 CRS, 20% experience maximum Grade 2, 12% experience maximum Grade 3, and 8% experience maximum Grade 4 versus a reduction in severity such that when treated with CTO1681, 30% of patients experience maximum of Grade 1, 10% experience maximum of Grade 2, and 10% experience maximum of Grade 3.

With a sample size of 104 patients (52 per group), there is over 65% power with a 1-sided type I error rate of 0.05 to detect a difference in proportion of 40% of control patients experiencing Grade ≥2 CRS versus 20% of CTO1681 patients.

Analysis Populations:

The modified Intent-to-Treat (mITT) Population will include all patients who receive both CAR T-cell therapy and at least 1 dose of study drug (CTO1681 or placebo). Patients will be grouped as randomized. A standard ITT population including all randomized patients may be analyzed as a test of mITT robustness.

The Safety Population will include all patients who receive at least 1 dose of study drug (CTO1681 or placebo). Patients will be grouped as treated.

The Per-protocol (PP) Population will include all patients in the mITT Population who received at least 5 doses of study treatment, CAR T-cell therapy, and did not have any major protocol deviations. Patients will be grouped as treated.

The Dose Limiting Toxicity (DLT) Population will include all patients in Phase 1b who receive both CAR T-cell therapy and at least 1 dose of CTO1681. Patients who withdraw before experiencing a DLT and before completing the DLT observation period (42 days following the first dose of study drug) for reasons unrelated to study procedures, conduct, or treatment, will not be included in this population.

Data Presentation/Descriptive Statistics:

All statistical analyses will be described in a detailed Statistical Analysis Plan (SAP) that will be executed prior to the unblinding of the Phase 2a data. In the event of discrepancies between the protocol and the SAP, the SAP will be used to determine correct analyses and methodologies.

Demographic, safety, PK, and efficacy parameters will be summarized by phase of study, presented overall for Phase 1b and by treatment group for Phase 2a, and supported by graphical presentations, as appropriate. Descriptive summaries will include n, mean, standard deviation, minimum, and maximum for continuous parameters and frequency and percentage for categorical parameters. Longitudinal data will be presented by appropriate time intervals depending on the endpoint being summarized.

Phase 1b: In keeping with the exploratory nature of a Phase 1 study, no inferential hypothesis testing will be performed. All endpoint summaries will be presented with descriptive statistics. Tabulations will be summarized by dose level and overall. Summaries will be performed on the mITT and DLT Populations. An end-of-Phase 1b analysis will be performed on completion of Phase 1b and before initiation of Phase 2a. This analysis will inform SRC recommendation and Sponsor decision on the RP2D, CTO1681 dose schedule, and CAR T-cell products to be included in Phase 2a.

Phase 2a: Primary and secondary endpoint analyses will be performed on the mITT and PP Populations. Analysis of safety parameters will be performed on the Safety Population.

The Phase 2a endpoint, rate, severity, onset, and duration of CRS, will be measured by the CRS grade AUC over the 30 days immediately following initiation of CAR T cell therapy. The AUC provides a single numeric value to represent the occurrence, duration, and severity of CRS. The average AUC will be compared between treatment groups using analysis of covariance (ANCOVA) accounting for randomization strata. The difference in mean comparison will be applied and a 1 sided p-value for the difference in AUC will be calculated. Further detail on this method will be provided in the SAP.

Rate, severity, onset, and duration of ICANS will be measured by the ICANS grade AUC over the 30 days immediately following initiation of CAR T-cell therapy. Other secondary endpoints (including ICANS grade AUC) will be analyzed using Cochran Mantel-Haenszel chi-square statistics for categorical variables and ANCOVA for continuous variables to account for randomization strata. Ordinal variables, including maximum severity of CRS, will be analyzed using a Wilcoxon-Mann-Whitney test overall and by stratum, as appropriate. Time-to-event endpoints will be compared using stratified logrank tests.

The robustness of CRS grade AUC will be established through correlation assessment of CRS grade AUC with the respective components. Specifically, graphical representations of the duration and severity versus AUC will be displayed. Tests for association will include differences in means t tests and receiver operating curves to determine, for example, if there are appropriate criteria for establishing the likelihood of a Grade 3 or higher CRS event through use of the AUC. In addition, the AUC will be assessed in relation to frequency and duration of hospitalizations and ICU admissions due to CRS and use of concomitant medications and anti-cytokine therapies for treatment of CRS to determine its relative association with healthcare usage. Similar analyses will be performed for the ICANS grade AUC. Additional information will be provided in the SAP.

Analysis of safety parameters will include summary of AEs, clinical laboratory measurements, vital signs, electrocardiogram results, and concomitant medications.

### Example 27: A Phase 1b/2a Study Methodology

A Phase 1b/2a study is designed to explore the safety and effectiveness of CTO1681 in preventing or reducing CAR T-cell-induced toxicities. The study will be separated into 2 distinct portions designated as Phase 1b and Phase 2a. After providing written informed consent for Phase 1b or Phase 2a of the study, the patient is considered enrolled. Study participants will be evaluated for inclusion during the Screening period. For all participants, the 19-day screening period will end 2 days before Baseline (Day −3).

Phase 1b: The Phase 1b portion of the study is a multicenter, open-label, dose-escalating, safety and pharmacokinetic (PK) study of multiple ascending doses of CTO1681 in patients with DLBCL who receive commercially available axicabtagene ciloleucel CD19-directed CAR T-cell therapy. This portion of the study will be conducted using a rolling 6 design to inform dose escalation among cohorts of patients with DLBCL. The Phase 1b portion of the study will include the following sequential steps for every patient in each cohort: Screening, treatment enrollment, pretreatment with lymphodepleting (LD) chemotherapy, initiation of study drug (CTO1681), CAR T-cell infusion, continuing treatment with CTO1681 (for a total of 15 days), and safety follow-up. Study Eligibility will be determined after leukapheresis and prior to LD chemotherapy. Timing of treatment with CTO1681 will occur according to the following schedule in relation to CAR T-cell infusion (CAR T-cell infusion is on “Day 0”):
Days −12 to −3: LD chemotherapy (if warranted per YESCARTA package insert [PI]).
Day −1: Initiate study drug (CTO1681) approximately 24 hours before CAR T-cell infusion.

Study drug will be continued orally (PO), 3 times daily (TID) for a total of 15 days. The total daily dose of CTO1681 will be 30, 60, or 90 μg (administered in TID increments of 10, 20 or 30 μg/dose, respectively), depending on the cohort into which each patient is enrolled. Study drug must be taken within 30 minutes after a meal or snack.

Day 0: CAR T-cell infusion (per YESCARTA PI).

Patients will be monitored to document tumor response for up to 6 months using the Lugano Criteria for Malignant Lymphoma. In addition, patients will be monitored throughout the study for efficacy and safety. A rolling 6 design will be implemented to advise on dose escalation. The dose-limiting toxicity (DLT) observation period is defined as 43 days following the first dose of study drug (Day −1) which is 42 days following initiation of CAR T-cell therapy (Day 0), that is, Day +41. Up to 3 dose cohorts will be considered with a maximum of 18 patients enrolled during the rolling 6 dose escalation procedure. Each dose level will be enrolled with a minimum of 3 and up to 6 patients.

A minimum of 3 patients must complete the DLT observation period (through Day +41) prior to data review for dose escalation. A maximum of 0/3, 0/4, 0/5 or 1/6 DLT may be recorded to allow escalation to the next higher dose level. Recommendations for next dose level will be made by the Safety Review Committee (SRC) based on safety, laboratory, and any PK and pharmacodynamic (PD) data that are available at the time of review, according to the rolling 6 design. If adequate safety data are not available from 1 or more patients in a given cohort, additional patients will be enrolled in that cohort, and only patients for whom there is adequate safety data may be included in the safety evaluation. If 1 DLT has been observed, any new patients will be entered at the same dose level until 6 patients are enrolled and have completed the DLT observation period, or at least 2 DLTs are observed, and therefore the maximum tolerated dose (MTD) has been exceeded. The MTD is defined as the maximum CTO1681 dose achieved where patients are treated safely. The dose may be escalated to the next level of dose if no more than 0 DLT observed in 3-5 patients or 1 DLT observed in 6 patients. If a dose level is too toxic (2 or more DLTs observed in 3-6 patients), then the MTD stopping rules for that cohort/dose level may be applied. Any or all of the cohorts deemed safe by the SRC following the dose escalation procedures may be backfilled up to a maximum of 12 patients at the discretion of the Medical Monitor and Sponsor. The backfill sample will allow further safety, antitumor response, PK, and PD information to better select a recommended Phase 2 dose (RP2D) at, or below the MTD for Phase 2a. The maximum sample size for Phase 1b is 36 patients. Table 3 below shows dose escalation and stopping rules.

TABLE 3
Dose escalation and stopping rules

| | Number of Patients | Dose | MTD | Maximum |
|---|---|---|---|---|
| Daily | in Rolling 6 Dose | Escalation | Stopping | Backfill |
| Dose Level | Escalation | Rules | Rules | Patients^{a} |
| 30 μg | 3-6 | 0/3 DLT | ≥2/3 DLT | 6-9 |
| | | 0/4 DLT | ≥2/4 DLT | |
| | | 0/5 DLT | ≥2/5 DLT | |
| | | ≤1/6 DLT | ≥2/6 DLT | |
| 60 μg | 3-6 | 0/3 DLT | ≥2/3 DLT | 6-9 |
| | | 0/4 DLT | ≥2/4 DLT | |
| | | 0/5 DLT | ≥2/5 DLT | |
| | | 1/6 DLT | ≥2/6 DLT | |
| 90 μg | 3-6 | 0/3 DLT | ≥2/3 DLT | 6-9 |
| | | 0/4 DLT | ≥2/4 DLT | |
| | | 0/5 DLT | ≥2/5 DLT | |
| | | ≤1/6 DLT | ≥2/6 DLT | |

Abbreviations: DLT=dose limiting toxicity; MTD=maximum tolerated dose; SRC=safety review committee; ^{a} Backfill patients will be enrolled at the discretion of the Medical Monitor and Sponsor if the dose level is determined safe according to the MTD stopping rules and by recommendation of SRC. A maximum of 12 patients may be treated at each dose level. A SRC will monitor the progress and safety of patients throughout the Phase 1b portion of the study and will determine if dose escalation may occur for each dose level cohort according to the rolling 6 design. In addition, the SRC will advise on the advancement of Phase 1b to the Phase 2a portion of the study and the selection of an RP2D on the basis of safety, including available antitumor response data. The SRC will also advise on the dose schedule and recommended CAR T-cell products for inclusion in Phase 2a.

Phase 2a: The Phase 2a portion of the study will be conducted as a randomized, double-blind, placebo-controlled study and will enroll approximately 100 patients. Patients will be treated during Phase 2a at the RP2D of CTO1681, which was established during Phase 1b, or with placebo. Patients will be randomized in a 1:1 ratio between the CTO1681 and placebo arms. Patients will receive commercially available axicabtagene ciloleucel CD19-directed CAR T-cell therapy. Each patient will follow the same study steps as noted above for the Phase 1b portion. Patients enrolled in this Phase 2a portion of the study will be monitored for tumor response for up to 12 months (using the Lugano Criteria for Malignant Lymphoma) following CAR T-cell infusion. Sparse PK samples will be collected in all patients in Phase 2a to perform population PK and exploratory exposure-response analyses.

For both the Phase 1b and Phase 2a portions of the study, safety, efficacy, PK, and PD assessments will be conducted at protocol-specified intervals. Study patients will be hospitalized for observation and management of CAR T-cell-related toxicities based on institutional guidelines. All CRS and ICANS grading will be according to American Society of Transplantation and Cellular Therapy (ASTCT) criteria. Detailed management guidelines for CRS and ICANS are provided in the full protocol and are consistent with standard of care (SOC) management.

An Independent Data Monitoring Committee (IDMC) composed minimally of 2 CAR T-cell/hematology oncology experts and an independent biostatistician will be established to monitor safety data during the Phase 2a portion of the study. The IDMC will perform planned safety data reviews after approximately 20, 40, 60, and 80 patients have been followed through Day +41, or have discontinued early from the study. Available data from Phase 1b will be included in each review. The study will be paused or stopped if any of the prespecified pausing or stopping rules are met.

Phase 1b and Phase 2a

For both the Phase 1b and Phase 2a portions of the study, safety, efficacy, PK and PD assessments will be conducted at protocol-specified intervals for Phase 1b, and Phase 2a.

The patient is planned to be treated as inpatient from Baseline/Day −1 to Day +6, at minimum, to allow for proper monitoring. During the period of Days +7 to +13, the patient may be discharged in the absence of CRS/ICANS at the discretion of the Investigator with the approval of the Example 28: Medical Monitor and Sponsor.

If the patient is discharged, then they must return to the clinic for a minimum of two visits during that week (Day +7 to Day +13), which must include the visit on Day +13) for AE collection and status assessment during the treatment period. In addition, study patients will be hospitalized for observation and management of CAR T-cell-related toxicities based on institutional guidelines. The currently commercially available CD19-directed CAR T-cell therapy for B-NHL (which includes DLBCL), axicabtagene ciloleucel (YESCARTA), will be utilized in both the Phase 1b and Phase 2a portions of this study.

### Example 29: Phase 1b—Safety Review Committee

The SRC will be composed of 1 or more study investigators, the study Medical Monitor, the Sponsor's medical representative(s) (or qualified delegate[s]), and ad hoc members as appropriate (for example, statistician, PK and regulatory affairs experts). The SRC will monitor the progress and safety of patients throughout the Phase 1b portion of the study. Regular systematic review of AEs will serve as the basis for pausing or prematurely stopping the study. Dose-limiting toxicities will be the primary criteria for pausing or stopping the study in alignment with the rolling 6 design operational characteristics. The SRC will carefully review any observed AE that may be considered a DLT that first presents more than 5 half-lives after discontinuation of study drug and evaluate if the AE might reasonably be considered related to the study drug; recommendations will appropriately reflect this consideration. The SRC will review the available safety, laboratory, electrocardiogram (ECG) and PK data through the completion of the DLT evaluation period (that is, Day +41, 43 days after the first dose of CTO1681). The SRC may recommend that the dose may be escalated or, additional patients may be enrolled (up to 6 per dose level), or the SRC may determine the MTD is reached, and no further dose escalation is appropriate. Cumulative safety data will be reviewed at each SRC meeting. After a dose level is determined safe according to the dose escalation procedures and SRC review of safety and PK data (if available), additional backfill patients may be enrolled (up to a maximum of 12 patients per cohort). A cohort will be paused or stopped if any of the pausing or stopping rules (per DLT criteria) are met. The SRC will advise on the advancement to the Phase 2a portion of the study and the selection of an RP2D based on available safety, antitumor response data, PK and PD results. An interim analysis summarizing the completed Phase 1b results will be performed. The SRC may also advise on any necessary protocol amendments with respect to patient safety throughout the Phase 1b portion of the study. Further details will be captured in a separate Safety Review Committee Charter.

### Example 30: Phase 2a—Independent Data Monitoring Committee

An IDMC minimally composed of 2 CAR T-cell/hematology oncology experts and an independent biostatistician will be established to monitor safety data during the Phase 2a portion of the study. The IDMC will perform planned safety data reviews after approximately 20, 40, 60, and 80 patients have been followed through Day +41, or have discontinued early from the study. The study will be paused or stopped if any of the prespecified pausing or stopping rules are met. The IDMC will carefully review any observed AE that may be considered a DLT that first presents more than 5 half-lives after discontinuation of study drug and evaluate if it might reasonably be considered related to the study drug; recommendations will appropriately reflect this consideration. An ad hoc meeting of the IDMC will be held in the event of a patient death. In addition, the IDMC will meet more frequently, as necessary, to review SAEs and provide recommendations on when to pause enrollment or terminate the study. The IDMC will also review available data regarding rates of CR in accordance with a prespecified set of nonbinding antitumor response impact assessments, planned to occur during the 40, 60, and 80 patient safety data reviews (20, 30, and 40 per treatment arm). Patients treated in Phase 1b at the R2PD will be included in these assessments. The antitumor response assessments will include all patients who have achieved CR, according to the Lugano Criteria, have a best antitumor response of progressive disease, have prematurely discontinued from the study, or have been followed for at least 3 months since first dose of study drug. Patients who have not achieved CR, have not had progressive disease, and have not been followed for a minimum of 3 months after treatment initiation will not be included in the analysis. This distinction is made to ensure there is sufficient follow-up to allow possible response or disease progression following treatment initiation to avoid an artificial under-reporting of response rate.

At each antitumor response impact assessment, for patients randomized to CTO1681, the CR rate and an exact Clopper-Pearson 95% CI will be computed to compare the upper bound of the CI to a 41% benchmark minimum rate derived from the lower boundary of CR rate 95% CI from the YESCARTA label. The first antitumor response assessment is expected to occur after 20 patients have been enrolled and treated with CTO1681; however, a minimum of 12 eligible patients are required to conduct analysis. Study enrollment may be paused for antitumor response data review as warranted.

### Example 31: Management of CRS and ICANS

CTO1681 should be continued in patients who develop CRS and/or ICANS, except as noted under the section for Management of DLTs. Detailed management guidelines for CRS and ICANS will be provided to the Investigator to standardize care across the participating study sites. These guidelines will be consistent with the SOC outlined in the PI for the selected CAR T-cell therapy, except that discretionary use of corticosteroids or tocilizumab prophylactically with Grade 1 CRS or ICANS will not be allowed. For Grade 2 or higher CRS and ICANS, treatment with corticosteroids and tocilizumab, as well as other supportive therapies may be considered.

### Example 32: Key Dose-Limiting Toxicities

A DLT is defined as a drug-related AE that is considered serious in nature, intolerable, or per the opinion of the SRC/IDMC, would place patients at a medical risk if a higher dose of CTO1681 were to be administered.

Key DLT criteria are:

Hypotension: Defined as documented, prolonged (≥2 hours) systolic blood pressure <80 mm Hg unresponsive to basic medical therapy (that is, fluid challenge).

Bleeding: Any clinically significant bleeding event (graded per Common Terminology Criteria for Adverse Events v5.0 [CTCAE]) Grade ≥3 (that is, excluding accidental cuts, periodontal, menstrual, or hemorrhoidal bleeding, or easily controlled epistaxis), including any episode of hemoptysis.

Hepatic dysfunction: Any occurrence of transaminase elevation of >5×upper limit of normal (ULN; CTCAE Grade ≥3).

QTc prolongation: Development of QT interval corrected using Fridericia's formula (QTcF) ≥550 msec or a >60 msec change from baseline, confirmed by repeat 12-lead electrocardiogram (ECG).

Ventricular tachycardia: CTCAE Grade ≥3 requiring urgent medical intervention.

Other: Safety data will be assessed for other CTCAE Grade ≥3 nonhematological toxicities in the treatment population that are potentially dose limiting and will include, but not be limited to:

Any Grade 3 or 4 toxicity and/or laboratory values involving vital organs (for example, cardiac or pulmonary) that result in significant and irreversible organ damage.

Any other CTCAE Grade ≥3 nonhematological toxicity that does not improve to CTCAE ≤Grade 2 within 72 hours.

Any death not attributed to underlying malignancy (Note: All CTCAE Grade 5 events will trigger a study pause to collect and assess the event for DLT determination. Expedited safety reporting to the Food and Drug Administration [FDA] would be included).

Exemptions from DLT criteria are based on anticipated high confounding prevalence in the specific study patient population and will include

Grade 3 or 4 anemia, lymphopenia, or thrombocytopenia.

Grade 3 or 4 fever or febrile neutropenia lasting ≤14 days.

Grade 3 or 4 tumor lysis syndrome lasting ≤14 days.

Any other laboratory finding that is not clinically significant and rapidly reversible to baseline of ≤Grade 2 within 14 days.

Grade 3 peripheral sensory neuropathy will not be considered a DLT in subjects with a prior history of peripheral sensory neuropathy.

Grade 3 renal and hepatic toxicity that improves to ≤Grade 2 within 7 days.

Grade 3 fatigue and anorexia that does not result in hospitalization, tube feeding, or total parenteral nutrition.

### Example 33: Objectives

Phase 1b:

Determine the preliminary safety profile of CTO1681 in patients with DLBCL receiving CAR T-cell therapy.

Determine the RP2D and, as appropriate, MTD of CTO1681 in patients with DLBCL receiving CAR T-cell therapy.

Phase 2a:

Determine preliminary CTO1681 effectiveness in preventing CRS or reducing CRS severity and duration in comparison to placebo.

Determine preliminary CTO1681 effectiveness in preventing ICANS or reducing ICANS severity and duration in comparison to placebo.

Determine the expanded safety profile of CTO1681 in patients with DLBCL receiving CAR T-cell therapy.

Investigate the potential impact of CTO1681 on antitumor activity of CAR T-cell therapy in comparison to historical data and placebo.

### Example 34: Endpoints

Phase 1b

Primary:

Adverse events (AEs), including DLTs, and laboratory and ECG abnormalities, as characterized by type, frequency, timing, severity (graded per CTCAE v5.0), seriousness, and relationship to CTO1681.

Exploratory:

Incidence of CRS (any grade).

Time to resolution of CRS (any grade).

Incidence of ICANS (any grade).

Time to resolution of ICANS (any grade).

Biomarkers relating to the mechanism of action (MOA) of CTO1681 and immune response.

Frequency and duration of hospitalizations and intensive care unit (ICU) admissions due to CRS or ICANS.

Use of tocilizumab, steroids, and other anticytokine therapies for treatment of CRS or ICANS (amount and duration).

PK concentration profile of CTO1681.

Levels and persistence of CAR T-cells.

Immune reconstitution (that is, B-cell aplasia).

Antitumor activity including complete response (CR), overall response rate (ORR), duration of response (DOR), and progression-free survival (PFS) according to Investigator review, and overall survival (OS).

Phase 2a (Active vs Placebo):

Primary:

Incidence of Grade ≥2 CRS.

Secondary:

Additional assessments of CRS incidence and severity:

Incidence of CRS (any grade).

Incidence of Grade ≥3 CRS.

Time to CRS resolution.

Additional assessments of ICANS incidence and severity:

Incidence of ICANS (any grade).

Incidence of Grade ≥2 ICANS.

Incidence of Grade ≥3 ICANS.

Time to ICANS resolution.

AEs and laboratory abnormalities, and ECG abnormalities as characterized by type, frequency, timing, severity (graded per CTCAE v5.0), seriousness, and relationship to CTO1681.

Frequency and duration of hospitalizations and ICU admissions due to CRS or ICANS.

Use of tocilizumab, steroids, and other anticytokine therapies for treatment of CRS or ICANS.

Antitumor activity including CR rate, ORR, DOR, and PFS according to Investigator review, and OS.

Exploratory:

Levels and persistence of CAR T-cells.

PK concentration profile of CTO1681.

Biomarkers relating to MOA of CTO1681 and immune response.

Immune reconstitution.

### Example 35: End of Study and Study Termination

End of study is defined as the time at which all patients have completed or discontinued treatment with CTO1681 (or placebo for the Phase 2a portion of the study) and have been followed for assessment of CAR T efficacy 6 or 12 months (Phase 1b or Phase 2a, respectively), or have died, been lost to follow-up, or withdrawn consent.

The study may be terminated at the discretion of the Sponsor if there is sufficiently reasonable cause. In the event of such action, written notification documenting the reason for study termination will be provided to each Investigator.

Circumstances that may warrant early termination of the study include, but are not limited to:

Determination of unexpected, significant, or unacceptable risk to patients.

Insufficient adherence to protocol requirements.

Plans to modify, suspend, or discontinue the development of study drug.

Failure to enroll patients at an acceptable rate after exhausting all efforts to improve enrollment.

Other administrative reasons.

Should the study be terminated prematurely, all study materials must be returned to the Sponsor or Sponsor designee.

### Example 36: Inclusion Criteria

1. Age 18 years or older.

2. Undergone leukapheresis and scheduled to receive protocol-specified commercially available axicabtagene ciloleucel CD19-directed CAR T-cell therapy for DLBCL without corticosteroid prophylaxis for CRS and/or ICANS. Patients eligible for study must have relapsed or refractory DLBCL after at least two prior lines of systemic therapy.

3. Met all inclusion criteria for CAR T-cell therapy per institutional guidelines.

4. Adequate organ function defined as: Serum creatinine ≤1.5×ULN and estimated glomerular filtration rate (eGFR) per Cockroft-Gault formula ≥60 mL/min×BSA m2/1.73 m2. Serum alanine aminotransferase/aspartate aminotransferase ≤2.5×ULN. Total bilirubin ≤1.5×ULN. Left ventricular ejection fraction ≥40% on echocardiogram or multigated acquisition scan and no clinically significant pericardial effusion. Platelets ≥50,000/mm3. Absolute neutrophil count >1000/μL. Absolute lymphocyte count >100/μL.

5. Documented measurable lymphoma disease adequate to judge by Lugano Criteria.

6. Eastern Cooperative Oncology Group performance status 0 to 1.

7. Female participants of childbearing potential and all male participants must agree to use Investigator-approved methods of birth control while on study drug and for 30 days thereafter.

8. Patients who are willing to provide written informed consent before the predose procedures, or patients who have a legal representative capable of providing informed consent on their behalf.

### Example 37: Exclusion Criteria

Patients meeting any of the following criteria will be excluded from the study:

1. Any cytotoxic chemotherapy within 14 days prior to leukapheresis.

2. Clinically significant malabsorption syndromes and swallowing difficulties which are inadequately controlled with medication (for example, odynophagia, dysphagia, gastroesophageal reflux disease) as per Investigator assessment.

3. Electrolyte imbalance that is unstable or unresponsive to therapy.

4. Clinically significant ECG abnormality at Screening or Baseline (Day −1) including, but not limited to, a confirmed QTcF value >470 msec. Patients to be excluded include those with QTcF readings that are borderline or difficult to interpret because of a condition such as bundle branch block, or in those where the end of the T wave is difficult to measure.

5. History of clinically significant arrhythmia and/or requiring anticoagulation/antiplatelet treatment at therapeutic dose.

6. Any clinically significant (that is, active) cardiovascular disease, including cerebral vascular accident/stroke (<6 months before enrollment), myocardial infarction (<6 months before enrollment) or unstable angina, and congestive heart failure ≥New York Heart Association Classification Class III.

7. Uncontrolled thromboembolic events or recent severe hemorrhage within the last 6 months.

8. Known history of any bleeding disorder.

9. Requirement for ongoing therapeutic doses of anticoagulant therapy, antiplatelet or fibrinolytic agents (low molecular weight heparin prophylaxis is allowed).

10. Baseline systolic blood pressure <100 mm Hg.

11. History of autoimmune disease/graft versus host disease requiring immunosuppressive therapy within the last 2 years. However, physiologic steroids (prednisone equivalent) may be given at a dose of 5 mg or less.

12. Patients who, in the opinion of the Investigator, would be unlikely to comply with study procedures or are otherwise unsuitable for enrollment.

### Example 38: Study Treatment

Lymphodepleting Chemotherapy

Lymphodepleting chemotherapy will be administered according to the CAR T-cell manufacturer's PI and institutional guidelines. Lymphodepleting chemotherapy is not required for patients with a white blood cell count ≤1,000 cells/μL within 1 week before planned CAR T-cell infusion but may be performed at the investigator's discretion.

Lymphodepleting chemotherapy will be supplied by the investigative site unless otherwise noted. Guidance on packaging, storage, preparation, administration, and toxicity management associated with the administration of LD chemotherapy should be followed as documented on the current product label.

Study Drug

For the Phase 1b portion of the study, study drug will consist of CTO1681-IR-OSD-10 μg Immediate Release Tablets.

For the Phase 2a portion of the study, study drug will consist of either CTO1681-IR-OSD-10 μg Immediate Release Tablets or matching placebo tablets. During Phase 1b of the study, CTO1681 will be administered PO as a 10-μg tablet. In Cohort 1, CTO1681 tablets will be administered PO TID for 15 days at a total daily dose of 30 μg. Cohorts 2 and 3 will receive CTO1681 PO TID 15 days at total daily doses of 60 and 90 μg, respectively. During Phase 2a of the study, CTO1681 will be orally administered as 10-μg tablets, potentially multiple tablets per dose, depending on the RP2D dose determined by the Phase 1b results. Placebo tablets will be identical in size, shape, color, and packaging to CTO1681 10-μg tablets.

### Example 39: Investigational Product, Dosage, and Mode of Administration

CTO1681 dosing must be initiated at the Baseline visit (Day −1), approximately 24 hours (±1 hour) before CAR T-cell infusion and will be dosed TID within 30 minutes after starting to eat a meal or snack; 5 hours (±30 minutes) preferred between meals on Day −1 to Day +13. Treatment compliance will be measured on all dosing days (Day −1 to Day +13). For Day +7 to Day +13, patients who are not hospitalized will be given a dosing diary to record any relevant information regarding the study drug, and the diary will be reviewed by site staff for compliance during inpatient visits, and required date recorded in the patients eCRF.

Phase 1b: CTO1681 will be orally administered as a 10-μg tablet. In Cohort 1, CTO1681 tablets will be administered PO TID for 15 days at a total daily dose of 30 ag. Cohorts 2 and 3 will receive CTO1681 PO TID for 15 days at total daily doses of 60 and 90 μg, respectively. Neither intrapatient dose modification nor dose modification within a cohort is permitted, except as specified in the management of DLTs.

Phase 2a: CTO1681 will be orally administered as 10-μg tablets, potentially multiple tablets per dose, depending on the RP2D dose determined by the Phase 1b results.

NOTE: As CTO1681 has a positive food effect, it should be administered within 30 minutes after completion of a meal or snack.

Duration of Study (per patient):

Phase 1b: approximately 9 months (including Screening).

Phase 2a: approximately 15 months (including Screening).

### Example 40: Reference Therapy, Dosage, and Mode of Administration

Phase 1b: None

Phase 2a: Placebo tablets will be administered PO, TID in a manner identical to active treatment. Placebo tablets will be identical in size, shape, color, and packaging to CTO1681 10-μg tablets.

### Example 41: CAR T-Cell Therapy

Patients will receive commercially available axicabtagene ciloleucel CD19-directed CAR T-cell therapy, which will be administered according to the manufacturer's PI and institutional guidelines.

### Example 42: Packaging and Labeling

CTO1681-IR-OSD-10 μg Immediate Release Tablets and placebo tablets will be provided in plastic bottles containing 45 tablets each. The study number will be printed on each individual drug label. Labels for Phase 1b will include the identification of the contents as only CTO1681-IR-OSD-10 μg Immediate Release Tablets will be distributed to patients during this open label portion of the study. Drug or placebo bottles will be properly masked as to contents during Phase 2a, the randomized part of the study.

Patient identification number, initials, and the date the bottle was dispensed for each patient will be completed by the study site staff or pharmacist. Packaging will meet all regulatory requirements.

### Example 43: Storage

Study drug tablets will be stored at room temperature (20° C. to 25° C. ([68° F. to 77° F.]) in their original container, according to the package label.

All study drug products will be stored in a secure, limited-access location and may be dispensed only by the Investigator or by a member of the staff specifically authorized by the Investigator.

### Example 44: Duration of Patient Participation

Patient participation during the Phase 1b portion of the study will be approximately 9 months, including Screening. Patient participation during the Phase 2a portion of the study will be approximately 15 months, including Screening.

### Example 45: Treatment Compliance

Patients participating in the Phase 1b of the study will be allotted 1, 2 or 3 bottles of CTO1681 10 μg tablets, for 30, 60 or 90 μg/day doses, respectively. In the Phase 2a portion, the CTO1681 or placebo assigned patients will receive 1, 2 or 3 bottles of either CTO1681 10 μg tablets or placebo depending on the randomization assignment and the RP2D carried forward in this portion of the study. Each bottle will contain 45 tablets of either CTO1681 10 μg tablets or placebo (Phase 2a only).

### Example 46: Prior and Concomitant Medications and Treatment

Prior and Concomitant Medications and Procedures

All medications administered and procedures conducted at study entry within 20 days before the first day of study drug administration (Day −1) and until the end-of-study visit (Month 6 or Month 12 for Phase 1b and Phase 2a, respectively) are to be recorded on the eCRF. In addition, all prior treatments for DLBCL should be recorded.

During the study, LD chemotherapy and CAR T-cell therapy are to be recorded on designated eCRF pages. All other medications taken from the time informed consent is signed should be recorded as concomitant medications on the appropriate eCRF page.

Prohibited Concomitant Therapy

The following concomitant therapies are prohibited during participation in the study:

Bridging chemotherapy is allowed, however, no additional therapy should be given within 7 days of initiation of LD chemotherapy.

No additional steroids should be given above physiologic dose after leukapheresis.

Systemic steroids and other immunosuppressive drugs for 3 months after CAR T-cell infusion unless used to manage CAR T-cell related toxicities.

Systemic steroids or tocilizumab for prevention of CRS/ICANS (prophylactic steroids or tocilizumab) or the treatment of Grade 1 CRS/ICANS. Steroids and/or tocilizumab should only be given for Grade 2 or higher CRS/ICANS.

Chemotherapy, immunotherapy, targeted agents, radiation, high-dose corticosteroids, and investigational agents other than LD chemotherapy are not allowed in this protocol except as needed for treatment of disease progression after CAR T-cell therapy.

Granulocyte macrophage colony-stimulating factor (GM-CSF) can potentially worsen CRS symptoms and should be avoided.

Short-acting granulocyte colony stimulating factor within 3 days of CAR T-cell infusion or long-acting granulocyte colony stimulating factor within 10 days of CAR T-cell infusion.

Central nervous system prophylaxis (for example, intrathecal methotrexate) must be stopped more than 1 week prior to CAR T-cell infusion.

Herbal and natural remedies.

Potential drugs that can interact with CTO1681 (CTO1681 [Investigators Brochure]).

### Example 47: Allowed Concomitant Therapy

Any medication or inactivated vaccine (including over-the-counter or prescription medicines and excluding vitamins) deemed for supportive care and safety of the patient received at the time of enrollment, or received during the study, must be recorded in the eCRF along with reason for use, dates of administration (including start and end dates), and dosage information (including dose and frequency). This may include, but is not limited to, the following:

CAR T-cell infusion routine prophylaxis medications (for example, diphenhydramine, acetaminophen/paracetamol).

Per study guidelines systemic steroids: dexamethasone, prednisone or other corticosteroids for the treatment of CRS or ICANS.

Physiologic steroid replacement (≤5 mg/day prednisone or equivalent) topical and inhaled steroids.

Symptomatic treatments of tumor-associated symptoms (for example, pain and nausea).

Oxygen therapy and blood products or transfusions.

Medications to treat chronic diseases (for example, hypertension and hypercholesterolemia).

Any concomitant medication not listed in the protocol may be considered on a case-by-case basis by the Investigator in consultation with the Sponsor or designee, if needed.

### Example 48: Management of Key Dose-Limiting Toxicities

The DLTs of hypotension, hepatic dysfunction, bleeding, QTc prolongation, and ventricular tachycardia will be managed as follows:

DLT of hypotension or hepatic dysfunction: CTO1681 will be interrupted until the DLT resolves. Should the DLT recur, CTO1681 will be discontinued for that patient.

DLT of bleeding: CTO1681 will be interrupted until the DLT resolves. Should the DLT recur, CTO1681 will be discontinued for that patient.

DLT of QTc prolongation or ventricular tachycardia: In the case of QTc prolongation, CTO1681 will be interrupted until the DLT resolves. Should the DLT recur, CTO1681 will be discontinued for that patient.

In the case of ventricular tachycardia, CTO1681 should be permanently discontinued for that patient. All other DLTs will be managed according to the institutional SOC.

### Example 49: Infection Prophylaxis

Patients should receive prophylaxis for infection with pneumocystis pneumonia, herpes virus, and fungal infections according to standard institutional practice or National Comprehensive Cancer Network guidelines.

Tumor Lysis Syndrome

All patients with significant malignancy burden and without a contradiction such as allergy should be started on prophylaxis (for example, allopurinol) as per institutional guidelines prior to CAR T-cell infusion. Prophylaxis should be discontinued once the risk of tumor lysis has passed.

B Cell Depletion

It is possible that B cell depletion and hypogammaglobulinemia will occur due to the effects of CAR T-cell therapy on normal B cells. Gammaglobulin will be administered for hypogammaglobulinemia according to institutional guidelines. At a minimum, trough immunoglobulin G (IgG) levels should be kept above 400 mg/dL, especially in the setting of infection (Hill, J. A., et al. (2019). Blood Rev 38: 100596.).

### Example 50: Cytokine Release Syndrome

The goal of CRS management is to prevent life-threatening conditions while preserving the benefits of antitumor effects. The CRS grading system created by the ASTCT and published by Lee et al. (Lee, et al. 2019 Biol Blood Marrow Transplant 25(4): 625-638) and treatment guidance for CRS is detailed in Table 4. General recommendations and monitoring for CRS are as follows:

Patients should be well-hydrated before and while receiving CTO1681.

CTO1681 should be administered in accordance with the study schedule and dosing instructions.

Patients should be monitored as inpatients during administration of CTO1681 from Day −1 to Day +13, except if discharged on Day +7 to +13 in the absence ofCRS/ICANS at the discretion of the Investigator with the approval of the Medical Monitor and Sponsor.

In the event symptoms (fever, hypotension, hypoxia) develop, the patient will be evaluated for/to rule out other causes (for example, infection). Other causes of symptoms should be treated as appropriate.

Neither steroids nor tocilizumab should be administered prophylactically. Table 4 shows management of CRS

TABLE 4
Management of Cytokine Release Syndrome

| | ASTCT CRS | |
|---|---|---|
| | Consensus Grading | CTA-2101 Treatment Guidelines |
| Grade 1 | Fever ≥38° C. | Continue administration of CTO1681. |
| | | No tocilizumab or steroids. |
| | | Empiric antibiotic coverage. |
| | | Maintain IV fluid hydration. |
| | | Acetaminophen PRN (not to exceed 3 g/24 h). |
| | | Hypothermia blanket PRN. |
| Grade 2 | Fever ≥38° C. | Continue administration of CTO1681. |
| | AND | Continue Grade 1 supportive care as |
| | Hypotension not | appropriate. |
| | requiring vasopressors | Utilize low-flow nasal cannula or blow-by |
| | AND/OR | oxygen for hypoxia. |
| | Hypoxia requiring low- | Administer IV fluids for hypotension. |
| | flow nasal cannula or | For hypotension that is refractory to fluid bolus, |
| | blow-by oxygen | administer tocilizumab 8 mg/kg IV over 1 hour |
| | | (not to exceed 800 mg). |
| | | If no clinical improvement in the signs and |
| | | symptoms of CRS after the first dose, repeat |
| | | tocilizumab every 8 h as needed. |
| | | Limit to a maximum of 3 doses of tocilizumab |
| | | in a 24-hour period; maximum total of 4 doses. |
| | | If hypotension persists after 1-2 doses of |
| | | tocilizumab, dexamethasone may be considered. |
| | | If improving, discontinue tocilizumab and |
| | | manage as Grade 1 above and quickly taper or |
| | | stop corticosteroids as clinically appropriate. |
| Grade 3 | Fever ≥38° C. | Continue administration of CTO1681. |
| | AND | Administer supportive respiratory care for |
| | Hypotension requiring | hypoxia as appropriate. |
| | vasopressors with or | Administer vasopressors for hypotension as |
| | without vasopressin | appropriate. |
| | AND/OR | Administer dexamethasone 10 mg IV every 8 to 12 h. |
| | Hypoxia requiring | If continued worsening, then consider next line |
| | high-flow nasal | of therapy: for example, anakinra, siltuximab, |
| | cannula, facemask, | high dose methylprednisolone, or other agents. |
| | nonrebreather mask, or | If improving, manage as appropriate grade above |
| | Venturi mask | and continue corticosteroids until the severity is |
| | | Grade 1 or less, then quickly taper or stop as |
| | | clinically appropriate. |
| Grade 4 | Fever ≥38° C. | Continue administration of CTO1681. |
| | AND | Administer vasopressors for hypotension and/or |
| | Hypotension requiring | supportive respiratory care for hypoxia as |
| | multiple vasopressors | appropriate. |
| | (excluding | Administer methylprednisolone 1000 mg IV |
| | vasopressin) | once per day for 3 days. |
| | AND/OR | If hypotension is refractory for >24 h or if |
| | Hypoxia requiring | patient is deteriorating rapidly, consider |
| | positive pressure | methylprednisolone 1000 mg 2-3 times a day or |
| | (CPAP, BiPAP, | additional therapy (for example, siltuximab |
| | intubation, mechanical | anakinra) per institutional guidelines. |
| | ventilation) | If improving, manage as appropriate grade above |
| | | and continue corticosteroids until the severity is |
| | | Grade 1 or less, then taper as clinically |
| | | appropriate. |

In Table 4:

Fever is defined as temperature ≥38° C. not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.

In Table 4, ASTCT=American Society of Transplantation and Cellular Therapy; BiPAP=bilevel/2-level positive airway pressure; C=Celsius; CPAP=continuous positive airway pressure; CRS=cytokine release syndrome; h=hours; IV=intravenous; PRN=as needed.

The CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as Grade 3 CRS.

Low-flow nasal cannula is defined as oxygen delivered at ≤6 L/minute. Low flow also includes blow-by oxygen delivery, sometimes used in pediatric. High-flow nasal cannula is defined as oxygen delivered at >6 L/minute.

### Example 51: Immune Effector Cell-Associated Neurotoxicity Syndrome

Like CRS, ICANS is a potentially life-threatening toxicity syndrome that is often caused by CAR T-cell therapy and frequently occurs within the first week following CAR T-cell therapy. ICANS can progress through increasingly severe manifestations. An ICANS grading system developed by the ASTCT and treatment guidance for ICANS is detailed in Table 5. If concurrent CRS is suspected, treat CRS according to the recommendations in Table 4.

TABLE 5
Management of ICANS

| | ASTCT ICANS Consensus | |
|---|---|---|
| | Grading | CTA-2101 Treatment Guidelines |
| Grade 1 | ICE score: 7-9 | Continue administration of CTO1681. |
| | Consciousness: Awakens | Administer levetiracetam for seizure |
| | spontaneously | prophylaxis. No tocilizumab or steroids. |
| | Seizure: None | Supportive care and neurological work-up |
| | Motor findings: None | including MRI and/or CT scan, EEG and |
| | Elevated ICP/cerebral | neurology consult per institutional protocols. |
| | edema: None | |
| Grade 2 | ICE score: 3-6 | Continue administration of CTO1681. |
| | Consciousness: Awakens to | Administer levetiracetam for seizure prophylaxis. |
| | voice | Administer dexamethasone 10 mg IV every 6 to 12 |
| | Seizure: None | h. |
| | Motor findings: None | If improving, continue corticosteroids until the |
| | Elevated ICP/cerebral | severity is Grade 1 or less, then quickly taper or |
| | edema: None | stop as clinically appropriate. |
| Grade 3 | ICE score: 0-2 | Continue administration of CTO1681. |
| | Consciousness: Awakens only to | Administer levetiracetam for seizure prophylaxis. |
| | tactile stimulus | Administer methylprednisolone 1000 mg IV once |
| | Seizure: Any clinical seizure | daily. |
| | focal or generalized that resolves | If improving, manage as appropriate grade above |
| | rapidly or nonconvulsive | and continue corticosteroids until the severity is |
| | seizures on EEG that resolve | Grade 1 or less, then taper or stop as clinically |
| | with intervention | appropriate. |
| | Motor findings: None | If cerebral edema is suspected (on head CT/MRI): |
| | Elevated ICP/cerebral | Consider methylprednisolone 1000 mg/ day in |
| | edema: Focal/local edema | divided doses for 3 days (edema in brainstem or |
| | on neuroimaging | thalamus); or 1 day (if other brain areas only). |
| Grade 4 | ICE score: 0 (patient is unable to | Continue administration of CTO1681. |
| | perform ICE) | Administer levetiracetam for seizure prophylaxis. |
| | Consciousness: Patient is | Administer methylprednisolone 1000 mg IV in 2 to |
| | unarousable or requires vigorous | divided doses daily. |
| | or repetitive tactile stimuli to | If improving, manage as appropriate grade above |
| | arouse. Stupor or coma | continue corticosteroids until the severity is Grade 1 |
| | Seizure: Life-threatening | less, then taper or stop as clinically appropriate. |
| | prolonged seizure (>5 min); | If Grade 4 ICANS is refractory for >24 h or if |
| | repetitive clinical or electrical | deteriorating rapidly, consider next line of therapy: |
| | seizures without return to | for example, anakinra, siltuximab, |
| | baseline in between | per institutional protocol. |
| | Motor findings: Deep focal | |
| | motor weakness such as | |
| | hemiparesis or paraparesis | |
| | Elevated ICP/cerebral edema: | |
| | Diffuse cerebral edema on | |
| | neuroimaging; decerebrate or | |
| | decorticate posturing; or cranial | |
| | nerve VI palsy; or papilledema; | |
| | Cushing's triad | |

In Table 5, ASTCT=American Society of Transplantation and Cellular Therapy; CT=computed tomography; EEG=electroencephalogram; h=hours; ICANS=immune effector cell-associated neurotoxicity syndrome; ICE=immune effector cell-associated encephalopathy; ICP=intracranial pressure; IV=intravenous; MRI=magnetic resonance imaging; VI=six.

In Table 5:

ICANS grade is determined by the most severe event (ICE score, level of consciousness, seizure, motor findings, raised ICP/cerebral edema) not attributable to any other cause; for example, a patient with an ICE score of 3 who has a generalized seizure is classified as Grade 3 ICANS.

A patient with an ICE score of 0 may be classified as grade 3 ICANS if awake with global aphasia, but a patient with an ICE score of 0 may be classified as Grade 4 ICANS if unarousable.

Depressed level of consciousness should be attributable to no other cause (for example, no sedating medication).

Tremors and myoclonus associated with immune effector cell therapies may be graded according to CTCAE v5.0, but they do not influence ICANS grading.

Intracranial hemorrhage with or without associated edema is not considered a neurotoxicity feature and is excluded from ICANS grading. It may be graded according to CTCAE v5.0.

Grading of CRS and ICANS will be conducted daily from Day 0 (and as clinically indicated, per ASTCT criteria.

For Day +7 to Day +13, if patients are not hospitalized, CRS/ICANS assessment will be performed only during clinic visits irrespective of timing of CTO1681 dose.

CRS Consensus Grading: In grading CRS, a CRS severity scale associated with antibody therapeutics was published by National Cancer Institute investigators. Appreciating the scale needed to be adapted for other therapeutics, to define mild, moderate, severe, and life-threatening events; account for overlapping symptoms; and guide treatment recommendations, a revised CRS grading system was created during a meeting supported by ASTCT and published by Lee et al. (Table 4).

ICANS Grading: Immune effector cell-associated neurotoxicity syndrome is a clinical neuropsychiatric syndrome that can occur following immunotherapy, particularly associated with T-cell engaging therapies and can often occur concurrently with or immediately after CRS. The ASTCT created and published an ICANS grading system (Table 5).

### Example 52: Fever and Neutropenia

Evaluation for the source of an infection should be performed per institutional guidelines. Fevers should be treated with acetaminophen and comfort measures. Nonsteroidal anti-inflammatory drugs and corticosteroids should be avoided. Patients who are neutropenic and febrile should receive broad-spectrum antibiotics. Maintenance intravenous fluids (normal saline) should be started on most patients with high fevers, especially if oral intake is poor or if the patient is tachycardic. An even daily fluid balance should be sought in patients who are not hypotensive and not experiencing active tumor lysis syndrome.

Filgrastim should be used according to published guidelines (for example, Infectious Disease Society of America).

### Example 53: Blood Product Support for Anemia and Thrombocytopenia

Treatment of anemia and thrombocytopenia should be provided per institutional guidelines. All blood products should be irradiated. Using complete blood counts as a guide, the patient should receive platelets and packed red blood cells as needed. Attempts should be made to maintain a hemoglobin level >8.0 μm/dL and a platelet count >20,000/mm3. Leukocyte filters should be used for all blood and platelet transfusions to decrease sensitization to transfused white blood cells and decrease the risk of Cytomegalovirus infection.

### Example 54: Deep Vein Thrombosis Prophylaxis

Prevention of deep vein thrombosis should be administered per institutional guidelines. Deep vein thrombosis prophylaxis should be administered to all patients with reduced mobility during hospitalization per institutional guidelines. Low molecular weight heparin is encouraged provided there are no contraindications (for example, recent surgery, bleeding diathesis, platelet count <50,000/μL) based on benefit/risk. Noninvasive mechanical intermittent pneumatic compression devices for deep vein thrombosis prophylaxis should be used in those who cannot receive anticoagulants due to increased bleeding risk or other concerns (Lyman, Carrier et al. 2021 Blood Adv 5(4): 927-974).

### Example 55: Criteria for Pausing or Stopping the Study

If any of the events referenced below as Pausing criteria or Stopping criteria occur within 43 days after the first exposure to the study drug (that is, Day +41), the SRC (in Phase 1b) or the IDMC (in Phase 2a) will be immediately informed, and an ad hoc meeting scheduled to review all available safety data. A pause to study recruitment will commence at that time. The committee will then provide recommendations as to how to proceed.

Pausing Criteria

Occurrence of any of the DLT criteria will prompt an ad hoc meeting of the SRC (Phase 1b) or IDMC (Phase 2a).

Stopping Criteria

The SRC (Phase 1b) or IDMC (Phase 2a) will recommend stopping the study if:

CTCAE Grade 4 toxicity (unmanageable, unexpected) occurring in >25% of dosed patients that is possibly, probably, or definitely related to CTO1681 (excluding fever, hematological toxicities, and CRS/ICANS).

Death resulting from a CTO1681-associated DLT not related to underlying disease, CAR T-cell therapy, or an extrinsic event (unrelated to the investigational product or study procedures). Specifically:

Fatal bleeding event.

Refractory hypotension resulting in death.

Refractory ventricular tachycardia resulting in death.

Note: Safety stopping criteria for Phase 2a may also take into account the data from subjects from Phase 1b who were treated at the RP2D as well as with lower dosages.

The Sponsor may decide to pause, stop, or make adaptations to the study based on recommendations of the SRC/IDMC and the totality of evidence. The SRC may recommend that the study be paused or stopped if it is determined that patient safety may be compromised by continuing the study. The IDMC may recommend that the study be stopped based on safety or reduced antitumor response.

Occurrence of the following will prompt an ad hoc meeting of SRC (Phase 1b) or IDMC (Phase 2a):

Hypotension: Defined as documented, prolonged (>2 hours) systolic blood pressure. <80 mm Hg unresponsive to basic medical therapy (that is, fluid challenge).

Bleeding: Any clinically significant bleeding event (graded per CTCAE v5.0) Grade >3 (that is, excluding accidental cuts, periodontal, menstrual, or hemorrhoidal bleeding, or easily controlled epistaxis), including any episode of hemoptysis.

Hepatic dysfunction: Any occurrence of transaminase elevation of >5× upper limit of normal (ULN; CTCAE Grade ≥3).

QTc prolongation: Development of QTcF >550 msec or a >60 msec change from baseline, confirmed by repeat 12-lead electrocardiogram (ECG).

Ventricular tachycardia: (CTCAE Grade >3) requiring urgent medical intervention.

Other: Safety data will be assessed for other CTCAE Grade >3 non-hematological toxicities in the treatment population that are potentially dose limiting and will include, but not be limited to:

Any Grade 3 or 4 toxicity and/or laboratory values involving vital organs (for example, cardiac or pulmonary) that result in significant and irreversible organ damage.

Any other Grade 3 non-hematological toxicity that does not improve to ≤Grade 2 within 72 hours.

Any death not attributed to underlying malignancy (Note: All Grade 5 events will trigger a study pause to collect and assess the event for DLT determination. Expedited reporting to the FDA would be included). SRC (Phase 1b) or IDMC (Phase 2a) will recommend stopping the study if:

CTCAE Grade 4 toxicity (unmanageable, unexpected) occurring in ≥25% of dosed patients that is possibly, probably, or definitely related to CTO1681 (excluding fever, hematological toxicities, and CRS/ICANS).

Death resulting from a CTO1681-associated DLT not related to underlying disease, CAR T-cell therapy, or an extrinsic event (unrelated to the investigational product or study procedures). Specifically:

Fatal bleeding event

Refractory hypotension resulting in death

Refractory ventricular tachycardia resulting in death

Note: safety stopping criteria for Phase 2a may also take into account the data from subjects from Phase 1b who were treated at the RP2D as well as with lower dosages. The Sponsor may decide to pause, stop, or make adaptations to the study based on recommendations of the SRC/IDMC and the totality of evidence. The SRC may recommend that the study be paused or stopped if it is determined that patient safety may be compromised by continuing the study. The IDMC may recommend that the study be stopped based on safety or reduced antitumor response.

### Example 56: Statistical Methods

Sample size considerations:

Phase 1b: The maximum sample size of 36 (maximum 12 per dose level) was selected to support the dose escalation and RP2D selection of CTO1681 dose level in accordance with the rolling 6 dose escalation procedure and backfill expansion to ensure sufficient safety data for Phase 2a dose selection.

Phase 2a: This study was designed with a sample size selected to provide sufficient power for the primary endpoint. With a sample size of 100 patients (50 per group), there is 80% power with a 1-sided type I error rate of 0.05 to detect a difference in proportion of 40% of control patients experiencing Grade ≥2 CRS versus 16% of CTO1681 patients.

Analysis Populations:

Modified Intent-to-treat (mITT) Population: The mITT Population will include all patients who receive both CAR T-cell therapy and at least 1 dose of study drug (CTO1681/placebo). Patients will be grouped by dose group for Phase 1b analyses and as randomized for the Phase 2a analyses.

Safety Population: The Safety Population will include all patients who receive at least 1 dose of study drug (CTO1681). Patients will be grouped as treated.

DLT Population: The DLT Population will include all patients in Phase 1b who receive both CAR T-cell therapy and at least 1 dose of CTO1681. Patients who withdraw before experiencing a DLT and before completing the DLT observation period (through Day +41, 43 days after the first dose of CTO1681 or placebo) for reasons unrelated to study procedures, conduct, or treatment, will not be included in this population. Additional analysis populations may be defined in the Statistical Analysis Plan (SAP). A standard intent-to-treat (ITT) population including all randomized patients may be analyzed as an evaluation of mITT robustness.

### Example 57: Study Rationale

CTO1681 has a broad impact on multiple (20+) cytokines to mitigate hypercytokinemia (CTO1681). Early administration of CTO1681, before the initial release of proinflammatory cytokines (for example, IFN-γ, TNF-α, IL-1, IL-2, and IL-6), may mitigate risk of CRS associated with CAR T-cell therapy. Through excess cytokine suppression, CTO1681 could potentially prevent the onset or escalation of CAR T-cell-induced immune-mediated toxicities, thereby reducing associated morbidities and hospitalization. The purpose of this study is to evaluate the safety and tolerability of CTO1681 and determine the preliminary effectiveness of CTO1681, measured mainly by reduction of CAR T-cell-induced toxicities, including CRS and ICANS and other efficacy measures. This Phase 1b/2a study will enroll adult patients with B-NHL (DLBCL subtype) who are scheduled to receive axicabtagene ciloleucel (that is, YESCARTA®) CD19-directed CAR T-cell therapy to manage potential variability between CAR T-cell therapy in response and adverse reaction rates. However, CRS and ICANS are not unique to axicabtagene ciloleucel, and CTO1681 studies in the future may involve additional CAR T-cell treatments. The Phase 1b portion will include an initial open-label, dose-finding, safety and toxicity assessment on a small sample (n=9 up to 36) of patients. The Phase 2a portion (n=100) will be a randomized, proof-of-concept assessment to investigate the potential for benefit of CTO1681, initiated prior to CAR T-cell therapy, versus placebo, in mitigating or reducing CAR T-cell-induced toxicity.

There is a medical need for alternative treatment options to tocilizumab and steroids in B-NHL with CRS and/or ICANS caused by CAR T-cell therapy. CTO1681 has demonstrated a tolerable safety profile in healthy volunteers, consistent with that reported for beraprost sodium. Results from a Phase 1a study in healthy volunteers support further investigation of CTO1681 to mitigate CAR T-cell therapy toxicities in the DLBCL patient population.

Rationale for Dose Selection and Schedule

The proposed doses for the Phase 1b multiple ascending dose portion of the study will be 10, 20, and 30 μg three times daily (TID) (30, 60, and 90 μg daily). The dose for the proposed Phase 2a portion of the study will be selected based on the findings from the Phase 1b portion of the study.

There is substantial evidence from the non-clinical and clinical use history of immediate release formulations of beraprost sodium, and the same active isomer as CTO1681, that these doses will be well tolerated and have the potential for efficacy in the prevention and treatment of CRS. Beraprost sodium contains 4 stereoisomers, one of which (314d) contains nearly all of the biological activity and is chemically identical to CTO1681. There is extensive worldwide clinical experience with beraprost sodium, and safe and effective doses for its non-CRS indications have been well described. In Japan, the approved doses of immediate-release formulations of beraprost sodium range from 60 to 180 μg/day (corresponding to 15 to 45 μg/day CTO1681), divided into 3 or 4 doses administered with food, and are often titrated upwards to tolerance. The safety of long-term dosing of higher levels of beraprost sodium, up to 480 μg/day (120 μg/day CTO1681) for up to 36 months, has been reported in studies of patients with pulmonary arterial hypertension (Vizza, Sciomer et al. 2001 Heart 86(6): 661-665, Galie, Humbert et al. 2002 J Am Coll Cardiol 39(9): 1496-1502, Nagaya, Shimizu et al. 2002 Heart 87(4): 340-345, Barst, McGoon et al. 2003 J Am Coll Cardiol 41(12): 2119-2125, Ono, Nagaya et al. 2003 Circ J 67(5): 375-378, Ono, Nagaya et al. 2003 Chest 123(5): 1583-1588, Durongpisitkul, Laoprasitiporn et al. 2005 Circ J 69(1):61-64, Park, Moon et al. 2006 Pulm Pharmacol Ther 19(4): 264-271, Vizza, Badagliacca et al. 2006 Cardiology 106(3): 168-173.).

Findings from the recently completed Phase 1 multiple ascending, repeat-dosing study of CTO1681, which evaluated doses of 15 to 60 μg/day in healthy volunteers over a period of 7 days, have confirmed this generally mild safety profile. No SAEs, treatment-emergent AEs (TEAEs) of special interest (defined as clinically significant changes in coagulation parameters or platelets, or persistent/recurrent symptomatic orthostatic hypotension), or TEAEs leading to study discontinuation or dose reduction occurred. Treatment-emergent AEs were exclusively mild (62 AEs) or moderate (4 AEs) in severity, and completely consistent with the safety profile reported prior for beraprost sodium and 314d.

Doses of beraprost sodium that are within the dose equivalencies of CTO1681 for the multiple ascending dose study have been shown to cause significant reductions in TNF-α in patients with diabetes (Fujiwara, Nagasaka et al. 2004 Exp Clin Endocrinol Diabetes 112(7): 390-394, Xu, Pan et al. 2020 Exp Ther Med 19(1): 639-645). It is hypothesized that similar reduction of TNF-α and other proinflammatory cytokines may mitigate the CRS frequently observed with CAR T-cell therapy. Importantly, no clinically significant decreases in cytokine levels (panel of 27 cytokines) were seen in healthy volunteers treated with CTO1681, suggesting that CTO1681 does not completely abolish cytokine responses, but rather mitigates excessive responses, further supporting the safety of CTO1681 and justifying continued clinical evaluation.

### Example 58: Efficacy Measurements

The primary Phase 2a endpoint, incidence of Grade ≥2 CRS, will be summarized as the frequency and proportion of patients with the event and compared between treatment groups using the Fisher's exact test assessed at a 1-sided type I error rate of 0.05. Exact 90% CI will be presented.

Incidence of CRS (any grade), Grade ≥3 CRS, ICANS (any grade), Grade ≥2 ICANS, and Grade ≥3 ICANS will be summarized in a similar manner to Grade ≥2 CRS. Comparisons between treatment groups will be made for exploratory purposes using Fisher's exact test.

Ordinal variables, including maximum severity of CRS, will be analyzed using a Wilcoxon-Mann-Whitney test, as appropriate. Time to CRS resolution and time to ICANS resolution will be analyzed using Kaplan-Meier methods and compared between treatment groups using a logrank test. Frequency and duration of hospitalization and ICU admissions due to CRS or ICANS, and use of tocilizumab, steroids, and anti-cytokine therapy for treatment of CRS or ICANs will be summarized using descriptive statistics.

### Example 59: Pharmacokinetic Measurements

Serial blood samples for PK assessments will be drawn for patients during the Phase 1b and Phase 2a portions of the study. Those samples will be used to determine the PK concentration profile of CTO1681.

In the Phase 1b portion, blood samples for preparation of plasma will be collected for analysis on:

Day −1 (first [morning] dose): at time=0 (before eating and within 1-hour prior to the morning dose), and 0.5 (±10 minutes), 1 (±10 minutes), 1.5 (±10 minutes), 2 (±10 minutes), 3 (±10 minutes), and 4 (±10 minutes) hours after the first (morning) dose of CTO1681.

Day −1 (second and third doses): at 0.5 hours (±10 minutes) predose and 1-hour postdose for the second and third doses of CTO1681. Day 0, Day +2, Day +4, Day +6 and Day +13: 1-hour (±10 minutes) after the first morning dose of CTO1681.

In the Phase 2a portion, sparse blood samples for the preparation of plasma will be collected for all patients to perform population PK and exploratory exposure-response analyses. Samples should be taken approximately 1 hour (±10 minutes) after the first morning dose. Where PK samples are scheduled at the same time as ECG assessments and cytokine draws, assessments should take place in the following order: (1) ECG measurements, (2) PK draw, and (3) cytokine draw.

Pharmacokinetic samples should be processed within 1 hour of collection and frozen at −80° C. until shipment. Further details regarding collection, handling and processing of PK samples will be contained in a separate study manual.

### Example 60: Biomarkers and Immune Reconstitution Measurements

For patients in the Phase 1b and Phase 2a portions of the study, biomarkers relating to the mechanism of action (MOA) of CTO1681 and immune response, as well as immune reconstitution will be collected through blood sampling.

Biomarkers relating to the MOA of CTO1681 and immune response include: cytokines (IFN-γ, TNF-α, IL-1, IL-2, IL-6, and GM-CSF), C-reactive protein (CRP), and ferritin. In addition, blood samples for IgG, a lymphocyte subsets enumeration (TBNK) panel and immunophenotyping will be performed to determine immune reconstitution, and will be obtained.

The objectives, assessments and analysis criteria of biomarkers and reconstitution analysis are defined below (Table 6):

TABLE 6
Objectives, assessments and analysis criteria of biomarkers and reconstitution analysis

| Objectives | Assessment | Analysis |
|---|---|---|
| Levels and persistence of | CAR copy number, | Level of CAR+ T-cells in serum post CAR T-cell |
| CAR T-cells | anti-CD19 CAR T, | infusion, maximum CAR+ T-cell level attained, |
| | CD19 | time to maximum level (peak), time at which |
| | | there were no detectable CAR+ T-cells, AUC of |
| | | CAR T-cell expansion from Day 0 to Day +30 |
| Immune reconstitution | IgG antibodies | Serum IgG levels over time (baseline to |
| | | last assessment) for example, <400 |
| Biomarkers relating to | Cytokines, ferritin, | Cytokines, ferritin, CRP, TBNK levels over |
| MOA of CTO1681 and | CRP, TBNK panel, | time (baseline to last assessment), change in |
| immune response | immunophenotyping | immunophenotype/peripheral blood |
| | | molecular characterization, composition of |
| | | T-cells, levels of normal B cells over time |

### Example 61: Cytokine Sampling

Patients should avoid strenuous physical activity for 24 hours prior to Cytokine collection. For both Phase 1b and Phase 2a, blood sampling for cytokines will occur pre- and approximately 1 hour post morning CTO1681 dose on Day +1 to Day +6. For Screening, LD chemotherapy, Day −1, Day 0, Day +7 to Day +13, Follow-up 1, Month 1, Month 3, Month 6, and Month 12 (Phase 2a only), sampling should occur at approximately the same time of day that the post-morning CTO1681 sampling will occur during the inpatient treatment period. On days where PK sampling is also scheduled for this timepoint (Days 0, +2, +4, +6 and +13), the cytokine sample should be drawn immediately following the PK sample. Samples should be processed within 1 hour of collection and frozen at −80° C. until shipment. Further details regarding collection, handling and processing of PK samples will be contained in a separate study manual.

### Example 62: Disease Assessment

Disease assessment will be performed by computerized tomography (CT) scan or positron emission tomography-CT scan. Screening scans are accepted if performed within the 30 days leading up to Screening visit. Magnetic resonance imaging as additional imaging will be performed only if deemed necessary per Investigator's assessment or institutional guidelines. If bridging therapy is administered, repeat imaging is required before LD chemotherapy.

### Example 63: Lugano Criteria for Malignant Lymphoma

The Lugano Criteria are the standard response criteria currently in use for lymphoma. The criteria are based on positron emission tomography (PET) or bidimensional tumor measurements on CT for non-fluorodeoxyglucose (FDG) avid lymphomas, or when PET imaging is not available (Cheson, Fisher et al. 2014 J Clin Oncol 32(27): 3059-3068). These criteria represent a revision of the Cheson criteria, published in 2014 (Barrington, Mikhaeel et al. 2014 J Clin Oncol 32(27): 3048-3058). The most important changes in classification criteria regarding imaging (Barrington, Mikhaeel et al. 2014, Cheson, Fisher et al. 2014) were:

Replacement of the dichotomous PET evaluation with the Deauville 5-point scale (5-PS).

Introduction of the interim PET scan during treatment.

The 5-PS is a purely visual qualitative assessment that allows a more differentiated classification than was possible with the previous method of evaluating FDG uptake. The criteria for the 5-PS are summarized in Table 6.

The interim PET examination makes it possible to adjust treatment earlier in some cases. This allows lack of treatment response to be addressed more quickly so that corresponding treatment escalation can be initiated or, in the case of early treatment response, treatment can be deescalated to minimize toxicity and secondary diseases (Spaepen, Stroobants et al. 2002 Ann Oncol 13(9): 1356-1363, Hutchings, Mikhaeel et al. 2005 Ann Oncol 16(7): 1160-1168, Hutchings, Loft et al. 2006 Blood 107(1): 52-59, Cerci, Pracchia et al. 2010 J Nucl Med 51(9): 1337-1343). These response criteria will be used to assess response in patients to CAR T-cell therapy (Table 7).

TABLE 6
Lugano criteria for malignant lymphoma

| Response | CT: Radiological Response | PET/CT: Metabolic Response |
|---|---|---|
| CR | All criteria met: | Score 1/2/3 according to the 5- |
| | No new lesions | without residual disease: |
| | Reduction ≤1.5 cm GTD of target | Score Criterion^{a} |
| | lesions | 1. No ¹⁸F-FDG uptake |
| | No extranodal manifestation | 2. Uptake ≤ mediastinum |
| | Spleen/liver: | 3. Uptake ≥ mediastinum |
| | Spleen and liver normal sized | and ≤liver |
| | New lesions: None | 4. Uptake moderately above |
| | | liver level |
| | | 5. Uptake significantly above liver |
| | | level and/or new lesion(s) |
| | | X New uptake region(s), |
| | | most likely not part of |
| | | New lesions: None |
| PR | Reduction of SPD of the max. 6 | Score 4/5 according to 5-PS with |
| | measurable target lesions | reduced ¹⁸F-FDG uptake with |
| | (nodal/extranodal) ≥50% | respect to baseline and residual |
| | Lesions that are too small/not | lymph nodes of any size |
| | measurable are assigned a size of | New lesions: none |
| | 0.5 × 0.5 cm | |
| | Lesions that are not visible | |
| | are assigned a size of 0 × 0 | |
| | cm Spleen/liver: | |
| | Length reduction >50% of the | |
| | enlarged spleen | |
| SD | Reduction of SPD of the max. 6 | Score 4/5 according to 5-PS |
| | measurable target lesions | New lesions: None |
| | (nodal/extranodal) <50% | |
| | Progressive disease criteria not | |
| | met New lesions: none | |
| Progressive | At least 1 criterion met: Abnormal | Score 4/5 according to 5-PS |
| disease | lymph nodes must meet the following | with increase in ¹⁸F-FDG |
| | criteria: | uptake with respect to |
| | GTD >1.5 cm and | baseline |
| | PPD increase ≥50% and | New lesions: New ¹⁸F-FDG-avid |
| | Increase of GTD or SAD by | typical lymphoma lesions |
| | 0.5 cm for ≤2.0 cm | |
| | 1.0 cm for >2.0 cm | |
| | Spleen/liver: | |
| | In the case of existing | |
| | splenomegaly: Increase in the size | |
| | of the spleen >50% | |
| | In the case of newly occurring | |
| | splenomegaly: Increase in the | |
| | length of the spleen ≥2.0 cm with | |
| | respect to baseline | |
| | New lesions: | |
| | Recurrence of lesions that had | |
| | already returned to the normal range | |
| | Or | |
| | New nodal lesion >1.5 cm | |
| | regardless of the axis | |
| | Or | |
| | New extranodal lesions >1.0 cm | |
| | regardless of the axis or ≤1.0 cm in | |
| | the case of clear assignment to | |
| | lymphoma | |

Abbreviations: 5-PS=5-point scale; CR=complete response; CT=computed tomography; FDG=fluorodeoxyglucose; GTD=great transverse diameter; PET=positron emission tomography; PPD=product of the perpendicular diameters; PR=partial response; SAD=short axis diameter; SD=stable disease; SPD=sum of the product of the diameters. ^{a}Deauville 5-PS

Overall Response Rate

Overall response rate is defined as the proportion of patients who achieved a confirmed best response of CR or partial response (PR) as their best response, as assessed by the Lugano Criteria (Table 7).

Duration of Response

Duration of response is determined for patients with an objective response. Duration of response is measured from the time response criteria are first met until the first date of recurrent or progressive disease, or death due to any cause.

Progression-free Survival

Progression-free survival is measured from the start of treatment (Phase 1b) or date of randomization (Phase 2a) until the first date of recurrent or progressive disease, or death due to any cause.

Overall Survival

Overall survival is defined as the time from start of treatment to the date of death due to any cause.

Levels and Persistence of CAR T-cells

Levels of CAR+ T-cells in serum post CAR T-cell infusion will be measured from Days 0 to 180 in the Phase 1b portion of the study, and Days 0 to 360 in the Phase 2a portion of the study.

### Example 64: In Vitro Analysis of CTO1681 Activity in CAR T-Cell Assay

The impact of CTO1681 on the efficacy of CD19-targeting CAR T-cells in vitro as well as its ability to reduce CRS-inducing pro-inflammatory cytokine levels in the presence of CAR T-cells and tumor cells in vitro were explored. The objective of the study was to determine if CTO1681 displayed an anti-CRS phenotype while preserving anti-tumor functions of CAR T-cells. CD3 T cells were sorted from one healthy donor PBMCs, stimulated for a short period of time and transduced with CD19 CAR-T lentivirus (LV) containing CD28 and CD3ζ domains. CD19 CAR T-cells were expanded for 6 days and used in the assay.

CD19-targeting CD3 T-cells were treated with 5 increasing concentrations of CTO1681 (0.36 nM, 1.8 nM, 9 nM, 45 nM, and 225 nM) or vehicle for 30 minutes, prior to, and during co-culture with CD19+Raji lymphoma target cells. Media alone, vehicle, and positive control (dexamethasone) wells were included. Following initial treatment, CAR T-cells (Effectors) were co-cultured with fluorescently labeled CD19+Raji tumor cells (Target cells) at 3 different Effector: Target cell ratios (10:1, 5:1 and 1:1) and treated with 5 different concentrations of CTO1681 or vehicle for 24 hours. Target cells (Raji cells) were fluorescently labelled with CPD (eBioscience™ Cell Proliferation Dye eFluor™) prior to co-culture to distinguish the Raji cells from the effector cells and allow for analysis of target viability by flow cytometry. Following incubation, supernatant was collected and levels of pro-inflammatory cytokines IL-6 and TNF-α were measured by multiplex Luminex assay and INF-7 was measured via time-resolved fluorescence resonance energy transfer (TR-FRET) assay. These cytokines were selected for quantification as they are early cytokines released following CAR T-cell infusion in vivo that causes hyperactivation of the immune system resulting in acute systemic inflammation, CRS, multiorgan failure, and possible death. In addition, flow cytometry was used to measure the level of Raji tumor/target cell death following CAR T treatment across CTO1681 conditions to measure impact of CTO1681 with CAR T-cell efficacy. Co-cultures were stained with viability dye 7-AAD for measuring viability.

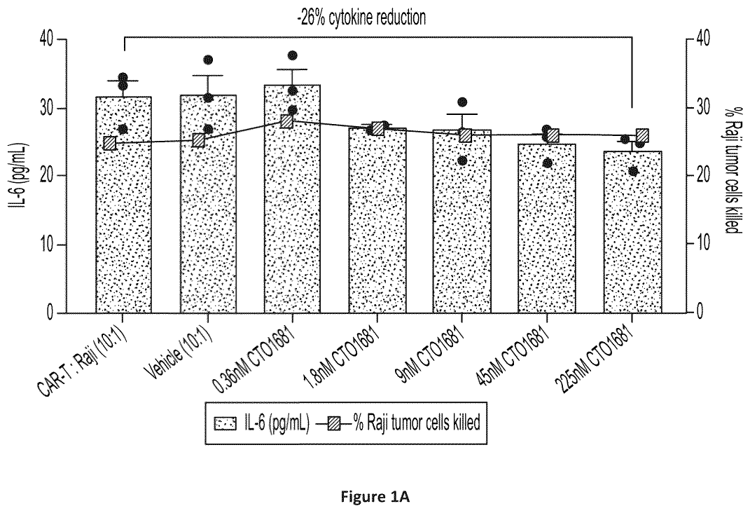

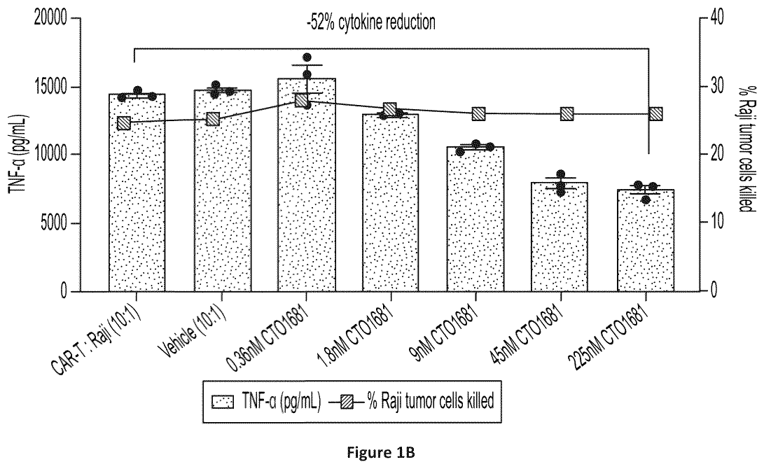

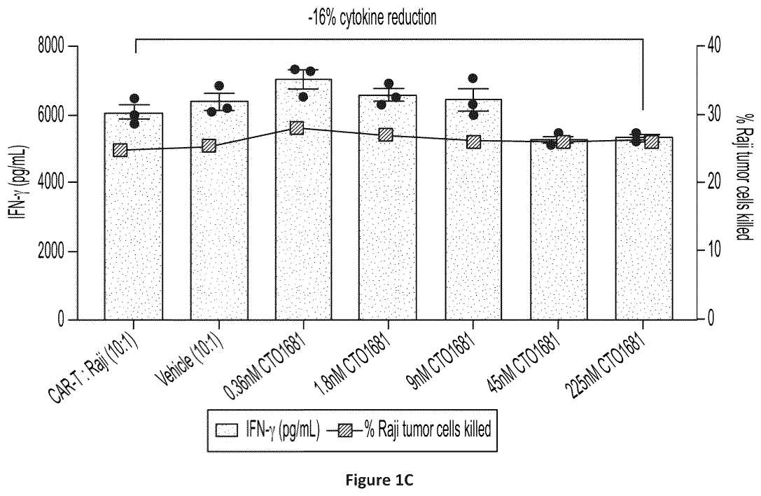

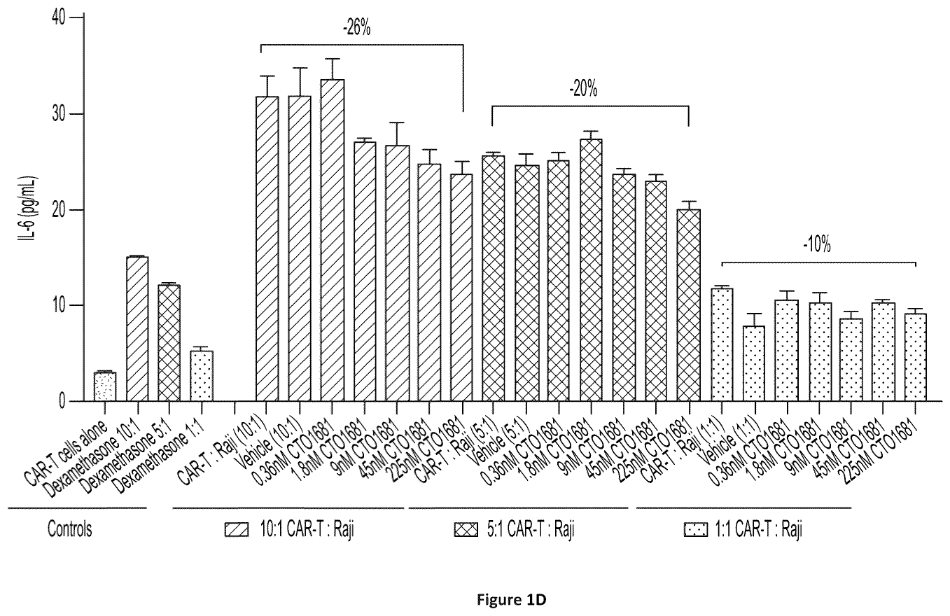

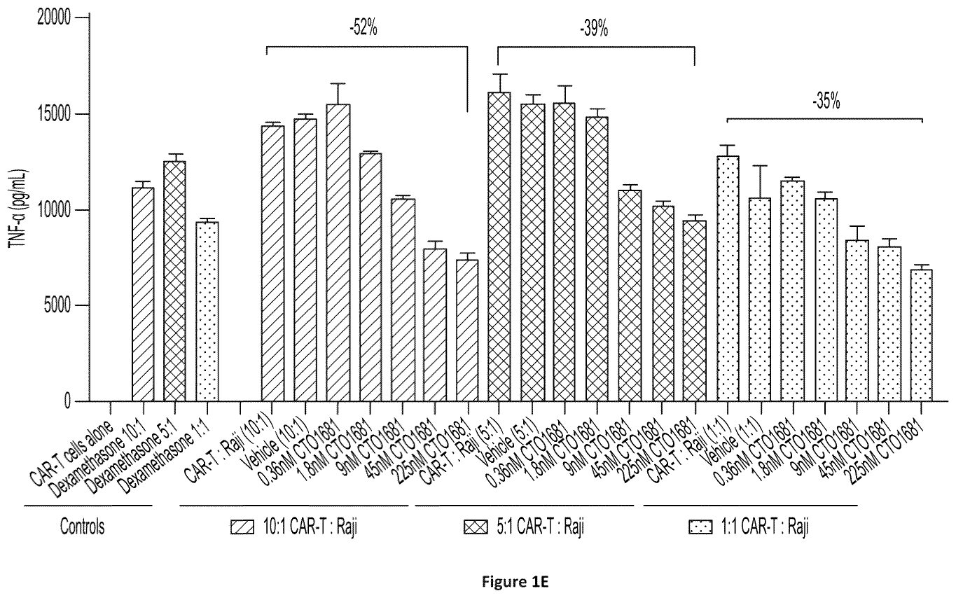

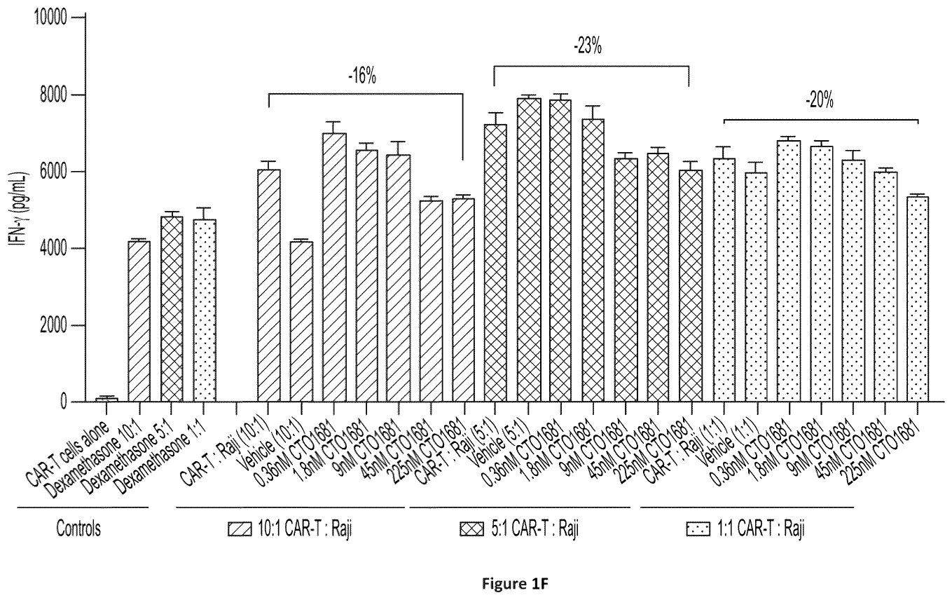

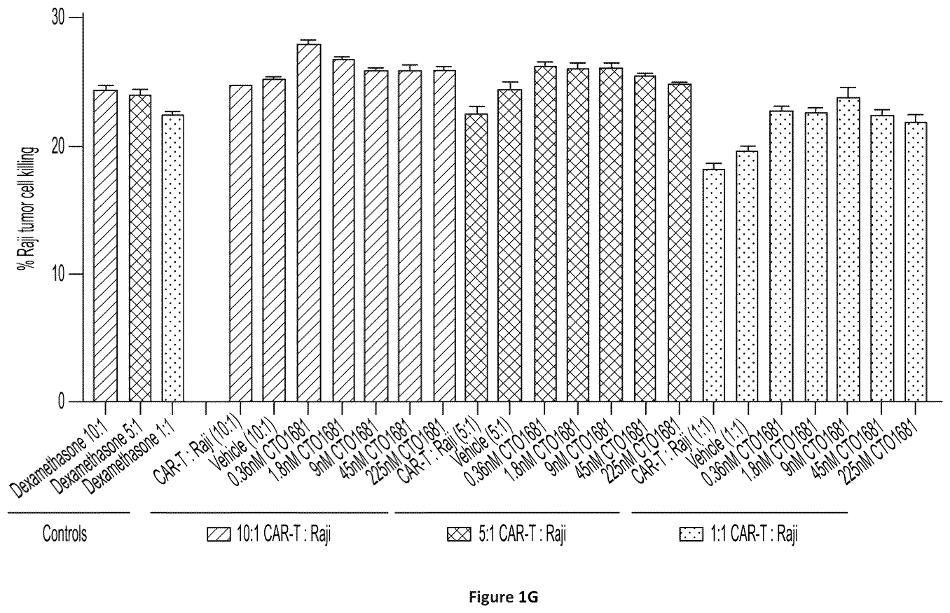

The results for 10:1 ratio of Effectors to Target cells are shown in FIG. 1A, FIG. 1B and FIG. 1C. Maximum reduction reached approximately 50% for TNF-α, 26% for IFN-γ, and 23% for TL-6, respectively. The study found that CTO1681 did not impact or interfere with the tumor/target cell killing effect of CD19-directed CAR T-cells in vitro (for example, CTO1681 does not interfere with CAR T-cell efficacy). All concentrations of CTO1681 showed target cell killing. As expected, CTO1681 dose-dependently reduced pro-inflammatory and CRS-inducing cytokine levels TL-6, TNF-α, and INF-7. Similar results were obtained at 5:1 and 1:1 ratios of Effectors to Target cells (FIG. 1D, FIG. 1E, FIG. 1F and FIG. 1G). Over a dozen cytokines have been shown to be elevated following CAR T-cell infusion in patients and CTO1681 has been shown to reduce the level of each cytokine in non-CAR T models. The results here demonstrating CTO1681 is able to significantly reduce the levels of IL-6, TNF-α, and IFN-7 aligns with previous findings that CTO1681 can reduce each of these cytokine levels in infectious disease models in vitro and in vivo. The results also provide additional validation that the CTO1681 targets an underlying source of pro-inflammatory cytokine release from immune cells. Therefore, CTO1681 is an optimal therapeutic candidate for CAR T-cell therapies. Collectively, results from this study demonstrate in vitro that CTO1681 does not interfere with CAR T-cell efficacy. It also provides strong evidence that CTO1681 acts on CAR T-cells to significantly reduce the release of pro-inflammatory cytokines critical in initiating a hyperactive immune response and life-threatening conditions including CRS.

### Example 65: In Vivo Effects of CTO1681 in CAR T-Cell Efficacy in NSG Tumor Bearing Mice

The effects if any, of CTO1681 on CAR T-cell activity in vivo were measured. Human T-cells expressing CD19-CD28-CD3Z CAR construct was used in this experiment.

6-7 week old NSG™ (NOD SCID gamma) mice were engrafted with 1×10⁶ luciferase (luc) expressing Raji tumor cells (herein referred to as “Raji-luc” tumor cells) each on Day 1 to allow for tumor growth over a 7-day period prior to the commencement of the treatment. CAR T-cell infusion was performed intravenously via lateral tail vein on (5×10⁶ CD19 CAR T-cells generated from healthy donors). NSG mice are severely immunodeficient, which facilitates ready tumor engraftment and subsequent clearance due to CAR T-cells can be measured.

Tumor growth after engraftment and subsequent clearance were monitored once weekly (starting on Day 7) using IVIS imaging to detect luciferase signal in living mice. CTO1681 administration began on Day 8, two days before CAR T-cell infusion, with injections twice a day for 14 days. Mice received total daily CTO1681 doses of 0.3 mg/kg/d, 0.2 mg/kg/d, or 0.15 mg/kg/d via two injections (BID). CTO1681 doses were selected based on the pharmacokinetic profile of the drug in mice. Control groups included mice receiving Raji-luc tumor cells without CAR T-cell treatment, mice receiving Raji-luc tumor cells and a CAR T-cell infusion, and mice receiving Raji-luc tumor cells with vehicle treatments. Mice were monitored for body temperature and health evaluations (weight, movement, posture, fur condition, etc.) throughout the entire study.

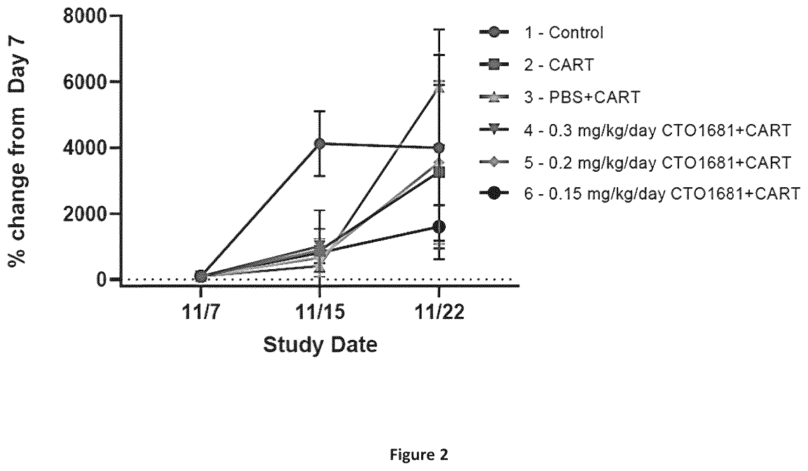

Tumor burden was confirmed prior to treatment commencement, with CTO1681 treatment groups starting BID dosing 48 hours prior to CAR T-cell administration. 2 way ANOVA for day 15 measurements found statistical difference (p=<0.0001) between Group 1 (no CAR-T treatment) and all other Groups. CD19 CAR-T cells significantly inhibited Raji tumor cell growth. All animals dosed with CD19 CAR-T cells including those that received a co-administration of CTO1681 had a significantly reduced tumor burden of between 3.9- and 9.5-fold increase. Hence treatment with CTO1681 did not affect CD19 CAR T-cell activity at any dose tested. No statistical difference (p=0.17 to 0.99) was found between Groups dosed with CAR-T cells alone (Groups 2 and 3) and animals dosed with both CAR-T cells and CTO1681 at any dose level. IVIS images and luciferin signal quantification are shown at Day 7 and Day 15 after allowing for suitable tumor growth post-engraftment, where Day 7 values represent pre-treatment baseline (FIG. 2). By Day 6 of CAR T-cell treatment (Day 15 of study), tumor burden was remarkably attenuated with no interference in CAR T-cell therapy associated reductions at all three CTO1681 dosages (FIG. 2). Follow-up observations at later timepoints showed disease progression in all experimental groups including those without CTO1681 treatment, indicating renewed replication and growth of Raji-luc cells not eliminated by CAR-T treatment, a phenomenon that is occasionally seen in the NSG tumor model. These data clearly show that CTO1681 administration together with CAR T-cell therapy does not interfere with treatment efficacy in vivo. Additionally, CTO1681 did not cause any increase in clinical morbidities, including weight loss or limb movement. In conclusion, CAR T-cell treatment with or without CTO1681 caused a drastic reduction in tumor burden as compared to mice who did not receive any treatment.

### Example 66: CTO1681 Cytokine Modulation

Peripheral blood mononuclear cells (PBMCs) from five donors, obtained from AllCells (Emeryville, CA, Catalog Number: PB001) were activated through TLR-3 using Poly r(I:C) expression. Cells were then incubated with beraprost (mixture of the four isomers) or with isomers BPS-314d (CTO1681, or Isomer A in the present Example), BPS-315l (Isomer B in the present Example), BPS-315d (Isomer C in the present Example), BPS-3141 (Isomer D in the present Example) at concentrations ranging from 0.3 nM to 750 nM. The samples were analyzed for TNFα production using a commercially available TNFα sandwich ELISA, and EC₅₀ values were calculated for each isomer. The data below clearly shows that isomer BPS-314d (Isomer A) is superior to the other three isomers tested.

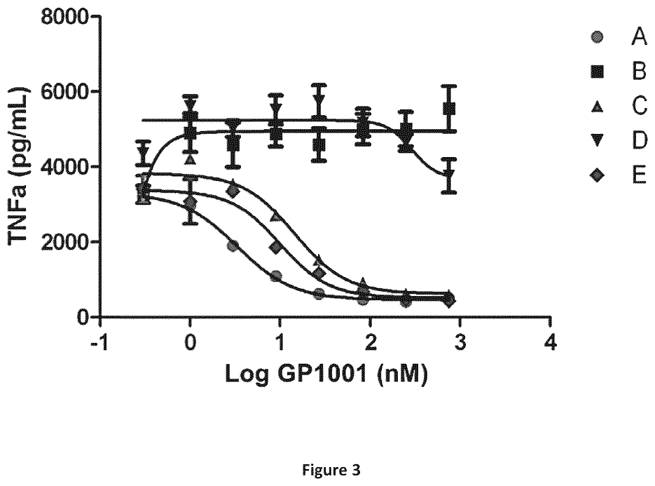

CTO1681 (BPS-314d or Isomer A) reduced TNFα production in activated PBMCs more than Isomer B, Isomer C, Isomer D or the parent mixture of the four isomers in three repeated experiments (FIG. 3). The EC50 for CTO1681 (BPS-314d or Isomer A) was significantly lower than that of the other isomers, with an EC₅₀ of 5.7 nM, compared to 33.59 nM for Isomer C in the first replicate (Table 7).

TABLE 7
EC50 Values Replicate 1

| | 95% Lower | | 95% Lower | |
|---|---|---|---|---|
| | Confidence | EC50 | Confidence | |
| Compound | Interval | (nM) | Interval | R² |
| Isomer A (CTO1681; | 3.17 | 5.7 | 10.53 | 0.83 |
| BPS-314d) | | | | |
| Isomer B (BPS-315l) | N/A | | | |
| Isomer C (BPS-315d) | 23.7 | 33.59 | 47.6 | 0.93 |
| Isomer D (BPS-314l) | N/A | | | |
| Beraprost | 13.34 | 17.11 | 21.95 | 0.95 |

EC₅₀ values for a second replicate corresponded to those of the first replicate, with an EC₅₀ of 4.141 nM for Isomer A compared to 21.71 nM for Isomer C (Table 8).

TABLE 8
EC50 Values Replicate 2

| | 95% Lower | | 95% Lower | |
|---|---|---|---|---|
| | Confidence | EC50 | Confidence | |
| Compound | Interval | (nM) | Interval | R² |
| Isomer A (CTO1681; | 3.429 | 4.141 | 5.001 | 0.96 |
| BPS-314d) | | | | |
| Isomer B (BPS-315l) | N/A | | | |
| Isomer C (BPS-315d) | 14.93 | 21.71 | 28.74 | 0.88 |
| Isomer D (BPS-314l) | N/A | | | |
| Beraprost | 5.236 | 8.64 | 14.26 | 0.84 |

EC₅₀ values for the third replicate also show a lower EC₅₀ for Isomer A than for the other isomers (Table 9).

TABLE 9
EC50 Values Replicate 3

| | 95% Lower | | 95% Lower | |
|---|---|---|---|---|
| | Confidence | EC50 | Confidence | |
| Compound | Interval | (nM) | Interval | R² |
| Isomer A (CTO1681; | 1.78 | 1.95 | 2.12 | 0.998 |
| BPS-314d) | | | | |
| Isomer B (BPS-315l) | N/A | | | |
| Isomer C (BPS-315d) | 13.2 | 22.6 | 32.0 | 0.978 |
| Isomer D (BPS-314l) | N/A | | | |
| Beraprost | 13.34 | 17.11 | 21.95 | 0.997 |

### Example 67: Pharmacokinetics in African Green Monkeys

The present Example presents the plasma concentrations of CTO1681 in African Green Monkeys (AGMs) after three times a day (TTD) dosing at varying drug concentrations. Here, increasing dose concentrations of CTO1681 correlate with increased plasma CTO1681 concentrations over multiple dose intervals. The results from this study collectively establish oral administration of CTO1681 in AGMs as a viable method to deliver CTO1681 systemically. In addition, the results from this study provide support for the tolerability of oral dosing of CTO1681, critical for the efficacy testing of CTO1681 in reducing cytokine levels and ARDS in this non-human primate model.

The data collected from this study demonstrate that the plasma concentration of CTO1681 in AGMs increases with respect to the concentration/dosage of CTO1681 administered TID via oral gavage. Overall, the maximum observed concentration measured after dosing (Cmax) and the area under the concentration versus time curve from the start of dosing to the last observed concentration calculated (AUC0-t) increase as the CTO1681 doses increase across rounds 1-3 (0.008, 0.04, and 0.2 mg/kg/day administered TID, respectively).

Study Design—CTO1681 PK murine (BALB/C) studies in a single dose PK study design demonstrated a T1/2 of approximately 1-2 hours. Similarly, murine (NSG) PK studies in a two times a day (BID) dose study design (CAS-2201) demonstrated a T1/2 of BID exposure to be approximately 5 hours while calculations of the elimination half-life using first daily dose and the measured exposures indicated 1.8 h using a range of 6-12 h (Rsq=0.40). Hence, the estimated half-life values of the BID and the single dose study are well-aligned PK parameters between the individual studies. With the knowledge of this short half-life for CTO1681, a study design with more frequent blood draws early post-injection was implemented in the study plans. Hence, due to the number and frequency of blood draws throughout the planned sampling schedule, and also due to the limitations of the allowable number of blood draws within a 24-hour window, a “nine representing three” animal sampling design was employed (i.e. three sampling cohorts of three each) over the time frame of blood draw in order to maximize the amount of blood draws within the 24-hour period of observation.

Individual Animal Metrics—Nine African green monkeys (approximately 16 years old, wild-caught) were used in this PK study. Per requirements of the CDC, all animals were quarantined at a CDC facility for 30 days followed by a 90-day quarantine/acclimation period at TNPRC prior to study initiation. Animals were dosed three times daily (TID) by oral gavage in three separate rounds of the study (ascending dose concentrations). Individual animal weights were tracked throughout the entirety of the study. Changes of individual animal weights over the period of study are reported in Table 10. On average, weights decreased 5.5% across all cohorts, with no individual animal losing more than 8.7% of its initial body weight over the course of the study. No difference in mean weight change is observed between male and female monkeys. Altogether, the individual weights of animals suggest that TID oral administration of CTO1681 does not cause significant weight loss. Ultimately, the animals handled the schedule well, all remaining in good health with a weight loss of no more than what was expected by the veterinary staff with the schedule intensity. CTO1681 was demonstrated to be overall tolerable for all animals in the study.

TABLE 10
Individual African green monkey weights
throughout entire observation period

| Animal | | Dose | Starting | End | Change in |
|---|---|---|---|---|---|
| ID | Sex | interval | Weight (kg) | weight (kg) | weight (%) |
| PI47 | FEMALE | 1 | 4.55 | 4.36 | −4.2 |
| PI51 | MALE | 1 | 7.21 | 6.79 | −5.8 |
| PI54 | MALE | 1 | 8.24 | 7.85 | −4.7 |
| PI46 | FEMALE | 2 | 4.83 | 4.45 | −7.9 |
| PI52 | MALE | 2 | 6.52 | 6.37 | −2.3 |
| PI55 | MALE | 2 | 6.22 | 5.68 | −8.7 |
| PI44 | FEMALE | 3 | 4.65 | 4.35 | −6.5 |
| PI53 | MALE | 3 | 5.97 | 5.73 | −4.0 |
| PI56 | MALE | 3 | 7.16 | 6.8 | −5.0 |

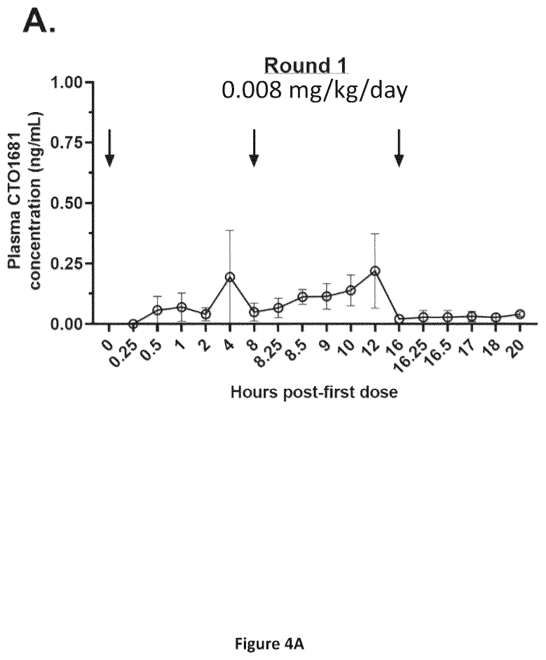

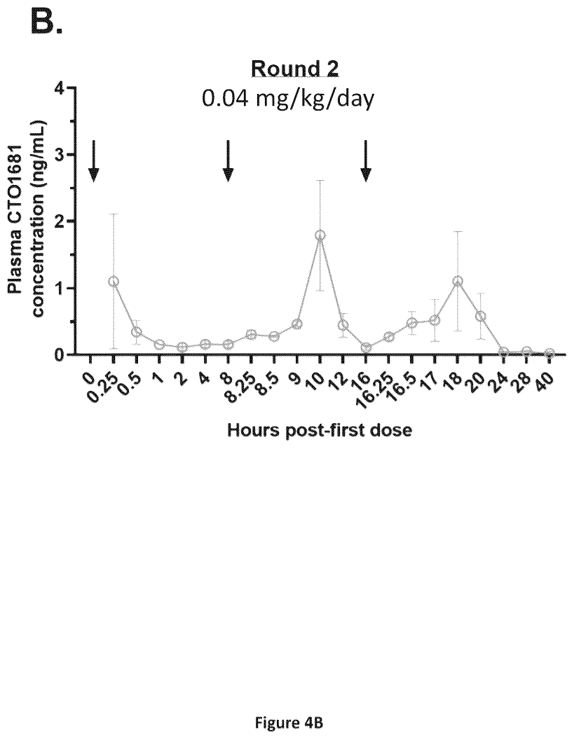

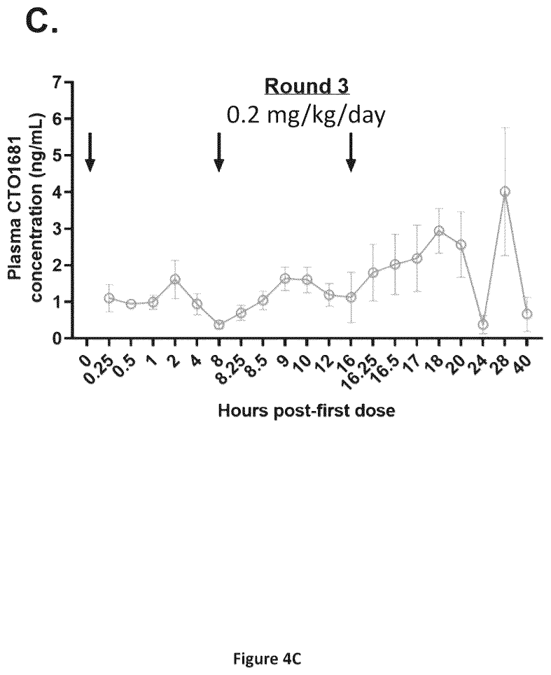

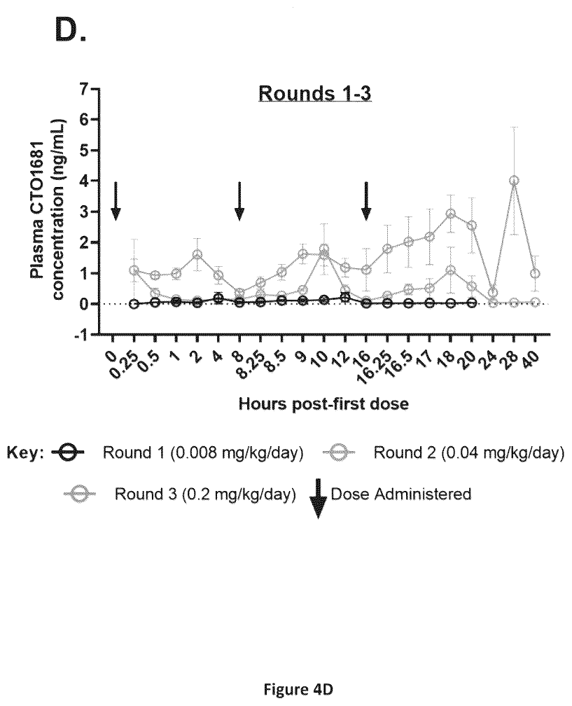

Pharmacokinetics of CTO1681 in AGM—Pharmacokinetic parameters were determined using a serial sampling design based on the individual plasma concentrations for each animal. Individual animal profiles were evaluated following each dose on a single day of dosing. Single day TID doses were administered using the same animals following a minimum of 7 days wash out between increasing dose level rounds, where Round 1 was TID 0.008 mg/kg/day (0.00267 mg/kg/dose), Round 2 was TID 0.04 mg/kg/day (0.0133 mg/kg/dose), and Round 3 was TID 0.2 mg/kg/day (0.0667 mg/kg/dose). Wash out (negative) plasma levels of CTO1681 between dose rounds were confirmed. Blood samples were collected from monkeys for determination of active pharmaceutical ingredient exposure of CTO1681 via femoral venipuncture sampling under anesthetic. Samples were collected at the following target timepoints relative to the first TID dose of the day: 0.25, 0.5, 1, 2, 4, and 8 hours (prior to second daily dose), 8.25, 8.5, 9, 10, 12, and 16 hours (prior to third daily dose), 16.25, 16.5, 17, 18, 20, 24, 28 and 40 hours after TID test item administration by oral gavage dosing, with the three daily doses administered at 0, 8 and 16 h in 9 animals, staggered for n=3 collection after administration of each daily dose. Plasma concentrations of CTO1681 were measured over the course of a 40-hour period beginning with the first TID dose administered at t=0 hours. Plasma CTO1681 was below the lower level of detection at t=0 for any animal across all dosing rounds. The average plasma CTO1681 concentrations over time are reported in FIG. 4 for dosing rounds 1, 2, and 3 independently (FIG. 4A, FIG. 4B, FIG. 4C) and combined (FIG. 4D), corresponding to 0.008, 0.04. and 0.2 mg/kg/day doses, respectively. The plasma levels of the lowest dose level (0.008 mg/kg/day) notably pushed the edges for the lower limits of CTO1681 quantitation. However, the concentration over time data suggests that plasma CTO1681 concentrations increase following each oral administration of the drug (indicated with arrows in FIG. 4). While inter-animal variability in CTO1681 concentrations was observed, the level of variability was within expected norms for the study design especially within wild caught animals and there is a clear dose-dependent increase in plasma drug concentration, indicated by increased plasma CTO1681 levels over rounds 1 to 3. During the third dosing period (t=16-40 hours), greater variability between individual animals was observed, which is hypothesized to be due to differences in drug administration by gavage from a different technician which occurred during late shifts (11:00 PM).

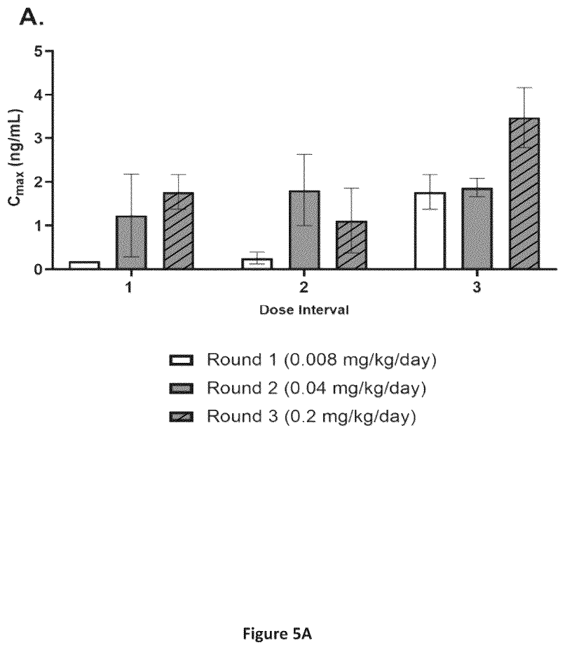

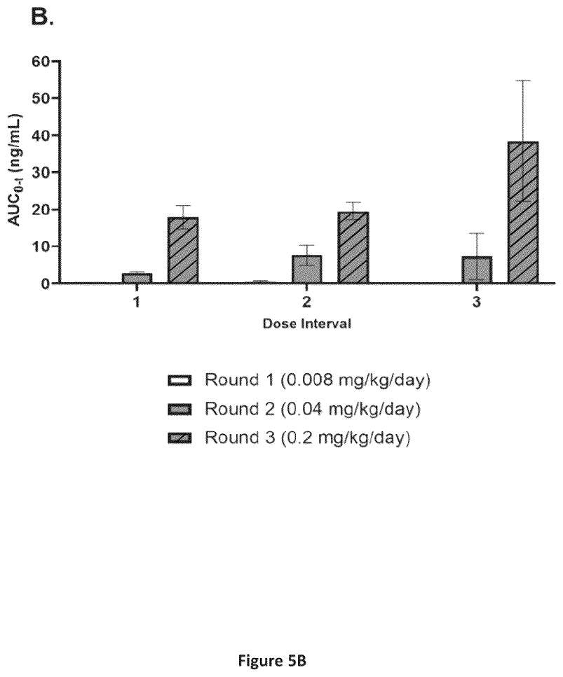

Standard PK parameters were measured as part of this study including the Cmax and AUC0-t (FIG. 5). An increase in average Cmax was measured over each dose interval across all dosing rounds with one exception of dose interval 2 in round 3 (FIG. 5A). Average Cmax levels at the lowest dose are more relatable to Cmax levels detected in human PK analysis in CTA-1901 and increased with dose levels. While there is variability between individual animals within dosing groups across rounds, a dose-dependent increase in both Cmax and AUC0-t was calculated over the last dose interval (dose interval 3; FIG. 5B). The highest CTO1681 dose (0.2 mg/kg/day) resulted in a maximum plasma Cmax of approximately 3 ng/mL following TID dosing. Additionally, the average time to reach maximum plasma CTO1681 concentration (Tmax) was positively correlated with dose concentration. Tmax across rounds 1,2, and 3 are calculated to be 1.5 hr, 2.0 hr, and 2.1 hr, respectfully. There is no correlation of Tmax across dosing intervals irrespective of administered dose.

These data collectively establish that oral administration of CTO1681 in African green monkeys' results in detectable and quantifiable dose dependent CTO1681 measurements and, on average, the greatest plasma concentration of CTO1681 reaches approximately 3 ng/mL following TID dosing at 0.2 mg/kg/day. Additionally, the average time to reach a maximum plasma CTO1681 concentration is positively correlated with drug dosage.

Summary—This work demonstrated that oral administration of CTO1681 via oral gavage allows for detectable and quantifiable plasma CTO1681. This study confirmed that plasma CTO1681 dose-dependently increases with increasing concentration of administered drug. Following TID dosing of 0.2 mg/kg/day CTO1681 (highest tested dose), the average Cmax measured was ˜3 ng/mL of plasma CTO1681 while the lowest dose is comparable to that detected in 60 ug dosing in normal healthy humans. Importantly, no toxicities or negative health effects, including significant weight loss, from CTO1681 administration were observed. Overall, this study establishes that oral administration of CTO1681 in African green monkeys is a viable model to study the efficacy of CTO1681 in a non-human primate model in subsequent studies.

### Example 68: Cytokine Measurements after CTO1681 Treatment in African Green Monkeys

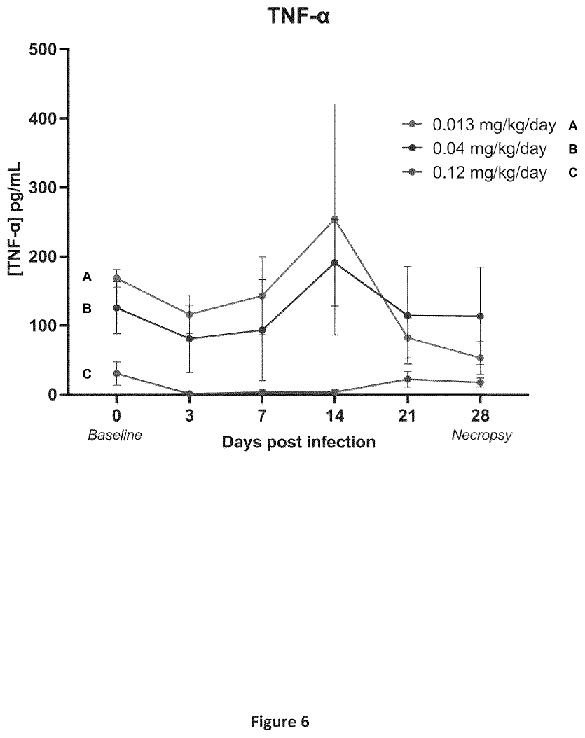

Longitudinal serum samples were taken through the course of the observation period. At the end of all three cohorts serum samples were utilized to determine circulating concentrations of 12 different cytokines. Representative data is pictured below for TNFα, a key central cytokine in CRS (FIG. 6). The AGM treated with the highest dose of CTO1681 demonstrated a remarkable and consistent reduction in cytokine levels as compared to the other two cohorts. Overall there appears to be a dose-dependent trend to cytokine reduction.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

What is claimed is:

1. A method of treating cytokine release syndrome (CRS), immune effector cell-associated neurotoxicity syndrome (ICANS), or both associated with CAR T-cell administration in a subject, the method comprising administering to the subject a population of CAR T-cells, and a first pharmaceutical composition; wherein:
the first pharmaceutical composition comprises at least an effective amount of beraprost, a beraprost isomer, or a pharmaceutically acceptable salt thereof; and
the first pharmaceutical composition does not reduce a cell killing mediated by the population of CAR T-cells by more than about 5%.

2. The method of claim 1, wherein the first pharmaceutical composition reduces levels of one or more of cytokines of IL-1α, IL-1β, IL-2, IL-4, IL-6, IL-7, IL-8, IL-10, IL-12, IFN-γ, TNF-α, IP-10, MCP-1, MIP-1, RANTES, and GM-CSF in the subject.

3. The method of claim 1, further comprising administering a second pharmaceutical composition comprising at least one corticosteroid, tocilizumab, IL-6 receptor blocker, or combinations thereof.

4. The method of claim 3, wherein the subject requires reduced treatment with the second pharmaceutical composition relative to a subject who does not receive the first pharmaceutical composition.

5. The method of claim 1, wherein the subject experiences reduced Parkinsonism effects upon receiving the first pharmaceutical composition relative to a subject who does not receive the first pharmaceutical composition.

6. The method of claim 1, wherein the subject experiences reduced severity measurements associated with CRS, ICANS or both upon receiving the first pharmaceutical composition relative to subject who does not receive the first pharmaceutical composition.

7. The method of claim 1, wherein the first pharmaceutical composition is administered once onset of CRS is detected by an increased level of one or more cytokines of IL-6, IL-10, IFN-γ, TNF-α, MIF, IL-5, IL-17A, IL-23, CXCL9/MIG, GCSF, VEGF-A, and TGF-β, or one or more of inflammatory biomarker C-reactive protein (CRP) and ferritin.

8. The method of claim 1, wherein the beraprost comprises at least one of BPS-314d, BPS-3141, BPS-315d, and BPS-315l.

9. The method of claim 1, wherein the isomer is BPS-314d (esuberaprost sodium salt).

10. The method of claim 1, wherein:
the CAR T-cell administration is performed to treat cancer; and
the cancer is B-cell lymphoma, aggressive, relapsed or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin lymphoma, non-Hodgkin lymphoma, acute myeloid leukemia, or multiple myeloma.

11. The method of claim 1, wherein the subject is a mammal.

12. The method of claim 1, wherein the first pharmaceutical composition is administered to the subject before the population of CAR T-cells are administered to the subject.

13. The method of claim 3, wherein the second pharmaceutical composition is administered to the subject after the population of CAR T-cells are administered to the subject.

14. The method of claim 1, wherein the first pharmaceutical composition is administered to the subject starting about 1 day, about 2 days, about 3 days, about 4 days, about 5 days, about 6 days, or about 7 days after the CAR T-cells are administered to the subject.

15. The method of claim 1, wherein the first pharmaceutical composition is administered for a period of about 1 day to about 30 days.

16. The method of claim 1, wherein the first pharmaceutical composition is administered starting one day before administration of the CAR T-cells and continued for a period of at least about 14 days.

17. The method of claim 1, wherein the administering comprises delivering the first pharmaceutical composition to the subject at an amount of beraprost, beraprost isomer, or a pharmaceutically acceptable salt thereof at least about 0.1 microgram.

18. The method of claim 1, wherein the population of CAR T-cells comprises a population of BCMA CAR-T cells, a population of CD19 CAR-T cells, a population of CD19-CD3 bispecific CAR-T cells, or combinations thereof.

19. A kit for treating a subject with CAR T-cells, the kit comprising:
a first container containing a first pharmaceutical composition comprising at least an effective amount of beraprost, beraprost isomer, or a pharmaceutically acceptable salt thereof;
a second container containing a second pharmaceutical composition comprising at least one corticosteroid, tocilizumab, IL-6 receptor blocker, or combinations thereof; and
instructions for the administration of the first pharmaceutical composition, the second pharmaceutical composition, and CAR T-cells to a subject.

20. The kit of claim 19, wherein the beraprost is BPS-314d (esuberaprost sodium salt).
